SEQUENCE LISTING (70 sequences)

SEQ ID NO: 1-36 - skipped (36 entries)

SEQ ID NO: 37 - protein, 12 aa, synthetic construct
SWELYYPLRANL

SEQ ID NO: 38 - protein, 5 aa, synthetic construct
HAVDI

SEQ ID NO: 39 - protein, 4 aa, synthetic construct
DGEA

SEQ ID NO: 40 - protein, 6 aa, synthetic construct
FYFDLR

SEQ ID NO: 41 - protein, 6 aa, synthetic construct
  SITE 3: note = 4-hydroxyproline
GFPGER

SEQ ID NO: 42 - protein, 4 aa, synthetic construct
VAPG

SEQ ID NO: 43-44 - skipped (2 entries)

SEQ ID NO: 45 - protein, 6 aa, synthetic construct
KQAGDV

SEQ ID NO: 46 - protein, 5 aa, synthetic construct
PHSRN

SEQ ID NO: 47 - protein, 4 aa, synthetic construct
REDV

SEQ ID NO: 48 - protein, 5 aa, synthetic construct
IKVAV

SEQ ID NO: 49 - skipped

SEQ ID NO: 50 - protein, 5 aa, synthetic construct
YIGSR

SEQ ID NO: 51 - protein, 7 aa, synthetic construct
SVVYGLR

SEQ ID NO: 52 - protein, 8 aa, synthetic construct
AEIDGIEL

SEQ ID NO: 53 - protein, 4 aa, synthetic construct
VTCG

SEQ ID NO: 54 - protein, 37 aa, synthetic construct
  SITE 19: note = 4-hydroxyproline
PGPPGPPGPPGPPGPPGFPGERGPPGPPGPPGPPGPP

SEQ ID NO: 55 - protein, 15 aa, synthetic construct
PHSRNGGGGGGRGDS

SEQ ID NO: 56 - protein, 17 aa, synthetic construct
SRARKQAASIKVAVADR

SEQ ID NO: 57 - protein, 6 aa, synthetic construct
KQLREQ

SEQ ID NO: 58 - protein, 5 aa, synthetic construct
SVTCG

SEQ ID NO: 59 - protein, 5 aa, synthetic construct
RGDSP

SEQ ID NO: 60 - protein, 6 aa, synthetic construct
GRGDSP

SEQ ID NO: 61 - skipped

SEQ ID NO: 62 - protein, 4 aa, synthetic construct
GRGD

SEQ ID NO: 63 - protein, 6 aa, synthetic construct
  VARIANT 1..3: note = This region may encompass 1-3 residues
GGGRGD

SEQ ID NO: 64 - protein, 8 aa, synthetic construct
  VARIANT 1..3: note = This region may encompass 1-3 residues
GGGRGDSP

SEQ ID NO: 65 - protein, 6 aa, Homo sapiens
LKRHQR

SEQ ID NO: 66-69 - skipped (4 entries)

SEQ ID NO: 70 - protein, 4 aa, synthetic construct
  VARIANT 1..4: note = This sequence may encompass 1-4 residues
GGGG

(19) United States
(12) Patent Application Publication
(10) Pub. No.: US 2025/0352573 A1
(43) Pub. Date: Nov. 20, 2025

(54) COVALENTLY CROSSLINKED POLYSACCHARIDES AND METHODS OF USE THEREOF

(71) Applicant: SIGILON THERAPEUTICS, INC., Cambridge, MA (US)

(72) Inventors: Matthew Buchanan, Atkinson, NH (US); Christopher A. Sparages, Cambridge, MA (US); Omar De Paolis, Boston, MA (US); Roger E. Harrington, Cambridge, MA (US); Weiheng Wang, Bedford, MA (US); Lauren E. Jansen, Roslindale, MA (US)

(21) Appl. No.: 18/880,149

(22) PCT Filed: Jun. 30, 2023
(86) PCT No.: PCT/US2023/026783

§ 371 Date: Dec. 30, 2024

Related U.S. Application Data
(60) Provisional application No. 63/357,894, filed on Jul. 1, 2022.
(60) Provisional application No. 63/452,091, filed on Mar. 14, 2023.

(51) Int. Cl. A61K 31/738; A61K 9/50; A61K 31/722; A61K 31/728; A61K 31/734; C08B 37/00; C08B 37/08; C08J 3/075

(52) U.S. Cl. CPC A61K 31/738; A61K 9/5036; A61K 31/722; A61K 31/728; A61K 31/734; C08B 37/003; C08B 37/0072; C08B 37/0084; C08J 3/075; C08J 2305/04; C08J 2305/08

(57) ABSTRACT

Described herein are hydrogel capsules (e.g., alginate hydrogel capsules) comprising polysaccharide polymers capable of covalent crosslinking to another moiety, such as another polysaccharide polymer; as well as related compositions and uses thereof.

### CLAIM OF PRIORITY

This application claims priority to U.S. Application No. 63/357,894 filed Jul. 1, 2022; and U.S. Application No. 63/452,091 filed Mar. 14, 2023. The disclosure of each of the foregoing applications is incorporated herein by reference in its entirety.

### BACKGROUND

The function of implanted devices depends in large part on the biological immune response pathway of the recipient (Anderson et al., Semin. Immunol. 20:86-100 (2008); Langer, Adv. Mater. 21:3235-3236 (2009)). Modulation of the immune response may impart a beneficial effect on the fidelity and function of these devices. As such, there is a need in the art for new compounds, compositions, and devices that achieve this goal.

### SUMMARY

Described herein are polymers (e.g., polysaccharide polymers) covalently crosslinked to another moiety, such as another polymer, as well as related compositions, hydrogel capsules comprising the same, and uses thereof. In an embodiment, the polymers (e.g., polysaccharide polymers) are crosslinked through one of the following methods: (i) a thiolene photoclick reaction; (ii) a Michael addition reaction; or (iii) an inverse electron-demand Diels Alder reaction. In an embodiment, the polysaccharide polymer comprises both a crosslinking moiety (e.g., a compound of Formula (IV) or (V)) and a compound of Formula (I), or a pharmaceutically acceptable salt thereof. These polysaccharide polymers can be incorporated into hydrogel capsules capable of encapsulating cells. The inclusion of a crosslinking agent into the polysaccharide polymers, and, in turn, the hydrogel capsules incorporating the polysaccharide polymers, may allow for tuning certain properties of the hydrogel capsules, including capsule diameter, stability, and integrity.

The details of one or more embodiments of the invention are set forth herein. Other features, objects, and advantages of the invention will be apparent from the Detailed Description, the Figures, the Examples, and the Claims.

### BRIEF DESCRIPTION OF THE DRAWINGS

FIGS. 1A-1C are graphs comparing the average fracture strength of exemplary dual crosslinked hydrogel capsules (e.g., hydrogel capsules comprising covalent crosslinking moieties and ionically crosslinked) described herein with ionically crosslinked hydrogel capsules.

FIG. 2 is a schematic depicting exemplary architecture of the polymers and related hydrogel capsules described herein.

### DETAILED DESCRIPTION

The present disclosure provides a polysaccharide polymer comprising a crosslinking moiety and a compound of Formula (I), as well as related compositions, hydrogel capsules comprising the same, and methods of making and use thereof.

### Abbreviations and Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” or “approximately” means when used herein to modify a numerically defined parameter (e.g., a physical description of a hydrogel capsule such as diameter, sphericity, number of cells encapsulated therein, the number of capsules in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter. Alternatively, particularly with respect to certain properties of the devices described herein, such as cell productivity, or density of the CBP or the afibrotic compound, the term “about” can mean within an order of magnitude above and below the recited value, e.g., within 5-fold, 4-fold, 3-fold, 2-fold or 1-fold.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer”, “administering”, or “administration”, as used herein, refer to implanting, absorbing, ingesting, injecting or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 3 is lower than the FBR induced by implantation of an afibrotic-null reference device, i.e., a device that lacks any afibrotic compound, but is of substantially the same composition (e.g., same CBP-polymer, same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule). In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered. In an embodiment, a cell is an immortalized cell or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). The CBP is less than 50, 40 30, 25, 20, 15 or 10 amino acids in length. In an embodiment, the CBP is between 3 and 12 amino acids in length, 4 and 10 amino acids in length, or is 3, 4, 5, 6, 7 8, 9 or 10 amino acids in length. The CBP amino acid sequence may be identical to the naturally-occurring binding domain sequence or may be a conservatively substituted variant thereof. In an embodiment, the CAM ligand is a mammalian protein. In an embodiment, the CAM ligand is a human protein selected from the group of proteins listed in Table 1 below. In an embodiment, the CBP comprises a cell binding sequence listed in Table 1 below or a conservatively substituted variant thereof. In an embodiment, the CBP comprises at least one of the cell binding sequences listed in Table 1 below. In an embodiment, the CBP consists essentially of a cell binding sequence listed in Table 1 below. In an embodiment, the CBP is an RGD peptide, which means the peptide comprises the amino acid sequence RGD (SEQ ID NO: 43) and optionally comprises one or more additional amino acids located at one or both of the N-terminus and C-terminus. In an embodiment, the CBP is a cyclic peptide comprising RGD (SEQ ID NO: 43), e.g., one of the cyclic RGD peptides described in Vilaca, H. et al., Tetrahedron 70 (35):5420-5427 (2014). In an embodiment, the CBP is a linear peptide comprising RGD (SEQ ID NO: 43) and is less than 6 amino acids in length. In an embodiment, the CBP is a linear peptide that consists essentially of RGD (SEQ ID NO: 43) or RGDSP (SEQ ID NO: 59).

TABLE 1
Exemplary CAM Ligand
Proteins and Cell Binding Sequences

| Protein | Cell Binding Sequence |
|---|---|
| E-cadherin | SWELYYPLRANL |
| | (SEQ ID NO: 37) |
| N-cadherin | HAVDI (SEQ ID NO: 38) |
| Collagen I | DGEA (SEQ ID NO: 39) |
| Collagen IV | FYFDLR (SEQ ID NO: 40) |
| | GFOGER (SEQ ID NO: 41) |
| | P(GPP)₅GFOGER(GPP)₅ |
| | (SEQ ID NO: 54) |
| | (O in SEQ ID NO: 41 and |
| | 54 is 4-hydroxyproline) |
| Elastin | VAPG (SEQ ID NO: 42) |
| Fibrinogen | RGD (SEQ ID NO: 43) |
| | GPR (SEQ ID NO: 44) |
| Fibronectin | RGD (SEQ ID NO: 43) |
| | KQAGDV (SEQ ID NO: 45) |
| | PHSRN (SEQ ID NO: 46) |
| | PHSRNGGGGGGRGDS |
| | (SEQ ID NO: 55) |
| | REDV (SEQ ID NO: 47) |
| Laminin | IKVAV (SEQ ID NO: 48) |
| | SRARKQAASIKVAVADR |
| | (SEQ ID NO: 56) |
| | LRE (SEQ ID NO: 49) |
| | KQLREQ (SEQ ID NO: 57) |
| | YIGSR (SEQ ID NO: 50) |
| Nidogen-1 | RGD (SEQ ID NO: 43) |
| Osteopontin | SVVYGLR (SEQ ID NO: 51) |
| Tenascin C | AEIDGIEL (SEQ ID NO: 52) |
| (TN-C) | |
| Tenascin-R | RGD (SEQ ID NO: 43) |
| Tenascin-X | RGD (SEQ ID NO: 43) |
| Thrombospondin | VTCG (SEQ ID NO: 53) |
| | SVTCG (SEQ ID NO: 58) |
| Vitronectin | RGD (SEQ ID NO: 43) |
| Von Willebrand | RGD (SEQ ID NO: 43) |
| Factor | |

“CBP-polymer”, as used herein, means a polymer comprising at least one cell-binding peptide molecule covalently attached to the polymer via a linker. In an embodiment, the polymer is not a peptide or a polypeptide. In an embodiment, the polymer in a CBP-polymer does not contain any amino acids. In an embodiment, the polymer in a CBP-polymer is a synthetic or naturally occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the C-terminus of an amino acid linker is joined to the N-terminus of the CBP and the N-terminus of the amino acid linker is joined to at least one pendant carboxyl group in the polysaccharide via an amide bond. In an embodiment, the structure of the linker-CBP is expressed as G₍₁₋₄₎-CBP, meaning that the linker has one, two, three or four glycine residues (“G₍₁₋₄₎” is disclosed as SEQ ID NO: 70). In an embodiment, one or more of the monosaccharide moieties in a CBP-polysaccharide, e.g., a CBP-alginate) is not modified with the CBP, e.g, the unmodified moiety has a free carboxyl group or lacks a modifiable pendant carboxyl group. In an embodiment, the number of polysaccharide moieties with a covalently attached CBP is less than any of the following values: 99%, 95%, 90%, 80%, 70%, 60%, 50%, 40% 30%, 20%, 10%, 5%, 1%.

In an embodiment, the density of CBP modification in the CBP-polymer is estimated by combustion analysis for percent nitrogen. In an embodiment, the CBP-polymer is an RGD-polymer (e.g., an RGD-alginate), which is a polymer (e.g., an alginate) covalently modified with a linker-RGD molecule (e.g., a peptide consisting essentially of GRGD (SEQ ID NO: 62) or GRGDSP (SEQ ID NO: 60)) and the density of linker-RGD molecule modification (e.g., conjugation density) is about 0.05% nitrogen (N) to 1.00% N, about 0.10% N to about 0.75% N, about 0.20% N to about 0.50% N, or about 0.30% N to about 0.40% N, as determined using an assay described herein. In an embodiment, the conjugation density of the linker-RGD modification in an RGD-alginate (e.g., a MMW alginate covalently modified with GRGDSP (SEQ ID NO: 60)) is 0.1 to 1.0, 0.2 to 0.8, 0.3 to 0.7, 0.3 to 0.6, 0.4 to 0.6 micromoles of the linker-RGD moiety per g of the RGD-polymer in solution (e.g., saline solution) with a viscosity of 80-120 cP, as determined by any assay that is capable of quantitating the amount of a peptide conjugated to a polymer, e.g., a quantitative peptide conjugation assay described herein. Unless otherwise explicitly stated or readily apparent from the context, a specifically recited numerical concentration, concentration range, density or density range for a CBP in a CBP-polymer refers to the concentration or density of conjugated CBP molecules, i.e., it does not include any residual free (e.g., unconjugated) CBP that may be present in the CBP-polymer.

“Cell-binding polypeptide (CBPP)”, as used herein, means a polypeptide of at least 50, at least 75, or at least 100 amino acids in length and comprising the amino acid sequence of a cell binding domain of a CAM ligand, or a conservatively substituted variant thereof. In an embodiment, the CAM ligand is a mammalian protein. In an embodiment, the CBPP amino acid comprises the naturally occurring amino acid sequence of a full-length CAM ligand, e.g., one of the proteins listed in Table 1, or a conservatively substituted variant thereof. “CBP-density”, as used herein, refers to the amount or concentration of a linker-CBP moiety in a CBP-polymer, e.g., an alginate modified with G₁₋₃RGD (SEQ ID NO: 63) or G₁₋₃RGDSP (SEQ ID NO: 64), unless otherwise explicitly stated herein.

“Cell-binding substance (CBS)”, as used herein, means any chemical, biological or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells, (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide or cell-binding polypeptide, as defined herein.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 2 below.

TABLE 2
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface Exposure | Asp, Glu, Asn, Aln, His, Arg, Lys |
| | Cys, Ser, Tyr, Pro, Ala, Gly, Trp, Tyr |
| | Met, Ile, Leu, Val, Phe |
| Secondary Structure Propensity | Ala, Glu, Aln, His, Lys, Met, Leu, Arg |
| | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a cell-binding peptide or a therapeutic protein that consists essentially of a recited amino acid sequence may also include one or more amino acids, including substitutions in the recited amino acid sequence, of one or more amino acid residues, which do not materially affect the relevant biological activity of the cell-binding peptide or the therapeutic protein, respectively. As another non-limiting example, a cell-binding peptide that consists essentially of a recited amino acid sequence may contain one or more covalently attached moieties (e.g., a radioactive or fluorescent label) that do not materially change the relevant biological activity of the cell-binding peptide, e.g., its ability to increase the viability or productivity of encapsulated cells as described herein.

“Crosslinked,” and variations thereof such as “crosslinking,” or “x-linked” as used throughout herein, refers to a chemical bond (e.g., ionic bond e.g., covalent bond) between two polymers. In some embodiments, when two or more chemical bonds are present, crosslinking refers to a mixture of both covalent and ionic bonds. In some embodiments, when two or more chemical bonds are present, crosslinking refers to different types of covalent bonds (e.g., covalent bonds comprising different or orthogonal functional groups). In some embodiments, when two or more chemical bonds are present, crosslinking refers to the same type of covalent bonds (e.g., covalent bonds comprising the same functional groups). In some embodiments, when two or more chemical bonds are present, crosslinking refers to the same type of ionic bonds (e.g., ionic bonds comprising the same ion, e.g., Ba²⁺).

“Derived from”, as used herein with respect to cells, refers to cells obtained from tissue, cell lines, or cells, which optionally are then cultured, passaged, differentiated, induced, etc. to produce the derived cells. For example, mesenchymal stem cells can be derived from mesenchymal tissue and then differentiated into a variety of cell types.

“Device”, as used herein, refers to any implantable object (e.g., a particle, a hydrogel capsule, an implant, a medical device) described herein. In some embodiments, the device contains cells (e.g., live cells) capable of expressing a therapeutic agent following implant of the device, and has a configuration that supports the viability of the cells by allowing cell nutrients to enter the device. In some embodiments, the device allows release from the device of metabolic byproducts and/or the therapeutic agent generated by the live cells.

“Differential volume,” as used herein, refers to a volume of one compartment within a device described herein that excludes the space occupied by another compartment(s). For example, the differential volume of the second (e.g., outer) compartment in a 2-compartment device with inner and outer compartments, refers to a volume within the second compartment that excludes space occupied by the first (inner) compartment.

“Effective amount”, as used herein, refers to an amount of a device, a device composition, or a component of the device or device composition, e.g, a plurality of hydrogel capsules comprising a cell, e.g., an engineered cell, or an agent, e.g., a therapeutic agent, produced by a cell, e.g., an engineered RPE cell, sufficient to elicit a biological response, e.g., to treat a disease, disorder, or condition. In some embodiments, the term “effective amount” refers to the amount of a component of the device, e.g., number of cells in the device, the density of an afibrotic compound disposed on the surface and/or in a barrier compartment of the device, the density of a CBS in the cell-containing compartment. As will be appreciated by those of ordinary skill in this art, the effective amount may vary depending on such factors as the desired biological endpoint, the pharmacokinetics of the therapeutic agent, composition or device (e.g., capsule, particle), the condition being treated, the mode of administration, and the age and health of the subject. An effective amount encompasses therapeutic and prophylactic treatment. For example, to mitigate the FBR, an effective amount of a compound of Formula (I) may reduce the fibrosis or stop the growth or spread of fibrotic tissue on or near the implanted device. An effective amount of a device, composition or component, e.g., afibrotic compound, may be determined by any technique known in the art or described herein.

An “endogenous nucleic acid” as used herein, is a nucleic acid that occurs naturally in a subject cell.

An “endogenous polypeptide,” as used herein, is a polypeptide that occurs naturally in a subject cell.

“Engineered cell,” as used herein, is a cell having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide. In an embodiment, an engineered cell comprises an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, an engineered cell can comprise an exogenous nucleic acid that controls the expression of an endogenous sequence. In an embodiment, an engineered cell comprises a polypeptide present at a level or distribution which differs from the level found in a similar cell that has not been engineered. In an embodiment, an engineered cell comprises an RPE engineered to produce an RNA or a polypeptide. For example, an engineered cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell (e.g., an RPE cell) comprises an exogenous nucleic acid sequence that comprises a chromosomal or extra-chromosomal nucleic acid sequence comprising a sequence that encodes a polypeptide, or which is expressed as a polypeptide. In an embodiment, the polypeptide is encoded by a codon optimized sequence to achieve higher expression of the polypeptide than a naturally-occurring coding sequence. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005). In an embodiment, an engineered cell (e.g., an RPE cell) comprises an exogenous nucleic acid sequence that modulates the conformation or expression of an endogenous sequence. In an embodiment, an engineered cell (e.g., RPE cell) is cultured from a population of stably-transfected cells, or from a monoclonal cell line.

An “exogenous nucleic acid,” as used herein, is a nucleic acid that does not occur naturally in a subject cell.

An “exogenous polypeptide,” as used herein, is a polypeptide that does not occur naturally in a subject cell, e.g., engineered cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Factor VII protein” or “FVII protein” as used herein, means a polypeptide that comprises the amino acid sequence of a naturally occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be produced by a device described herein, e.g., a device containing engineered RPE cells, include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255. Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising tissue factor (TF) embedded in a lipid membrane and Factor X (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally occurring factor VIII polypeptide or variant thereof that has an FVIII biological activity, e.g., coagulation activity, as determined by an art-recognized assay, unless otherwise specified. FVIII proteins that may be expressed by a device described herein, e.g., a device containing engineered RPE cells, include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the cells are engineered to encode a precursor factor VIII polypeptide (e.g., with the signal sequence) with a full or partial deletion of the B domain. In an embodiment, the cells are engineered to encode a single chain factor VIII polypeptide which contains a variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.).

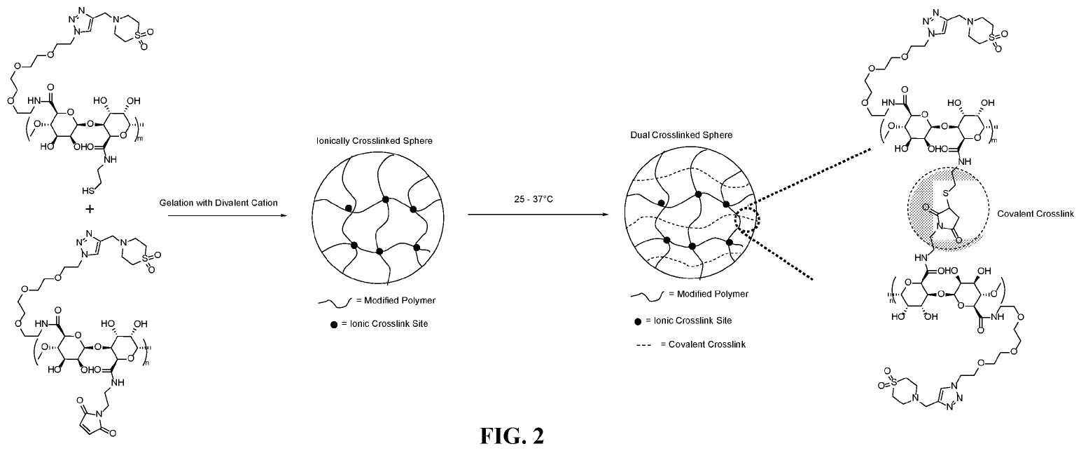

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1); U.S. Pat. No. 5,171,844 (e.g., col. 4, line 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39). In some embodiments, a FVIII-BDD protein produced by a device described herein (e.g., expressed by engineered cells contained in the device) has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, e.g., LKRHQR (SEQ ID NO: 65) at amino acids 1643-1648, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution of one or more amino acids in the furin protease recognition sequence (LKRHQR (SEQ ID NO: 65) at amino acids 1643-1648) that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be produced by devices described herein (e.g., a device containing engineered RPE cells) include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, cells are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2 032 607, thrombin generation time assay (TGA) and rotational thromboelastometry, e.g., as described in WO 2012/006624.

A number of functional FIX variants are known and may be expressed by engineered cells encapsulated in a device described herein, including any of the functional FIX variants described in the following international patent publications: WO 02/040544 at page 4, lines 9-30 and page 15, lines 6-31; WO 03/020764 in Tables 2 and 3 at pages 14-24, and at page 12, lines 1-27; WO 2007/149406 at page 4, line 1 to page 19, line 11; WO 2007/149406 A2 at page 19, line 12 to page 20, line 9; WO 08/118507 at page 5, line 14 to page 6, line 5; WO 09/051717 at page 9, line 11 to page 20, line 2; WO 09/137254 at page 2, paragraph [006] to page 5, paragraph [011] and page 16, paragraph [044] to page 24, paragraph [057]; WO 09/130198 A2 at page 4, line 26 to page 12, line 6; WO 09/140015 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXFc-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:2), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function). “Interleukin-2 protein” or “IL-2 protein”, as used herein means a polypeptide comprising the amino acid sequence of a naturally-occurring IL-2 protein or variant thereof that has an IL-2 biological activity, e.g., activate IL-2 receptor signaling in Treg cells, as determined by an art-recognized assay, unless otherwise specified. IL-2 proteins that may be produced by a device described herein, e.g., a device containing engineered RPE cells, include wild-type primate (e.g., human), porcine, canine, and murine proteins, as well as variants of such wild-type proteins. A variant IL-2 protein preferably has at least 50%, 75%, 90% or more (including >100%) of the biological activity of the corresponding wild-type IL-2. Biological activity assays for IL-2 proteins are described in U.S. Pat. No. 10,035,836, and include, e.g., measuring the levels of phosphorylated STAT5 protein in Treg cells compared to CD4+CD25−/low T cells or NK cells. Variant IL-2 proteins that may be produced by a device of the present disclosure (e.g., a device containing engineered RPE cells) include proteins with one or more of the following amino acid substitutions: N88R, N88I, N88G, D20H, Q126L, Q126F, and C125S or C125A.

“Islet cell” as used herein means a cell that comprises any naturally occurring or any synthetically created, or modified, cell that is intended to recapitulate, mimic or otherwise express, in part or in whole, the functions, in part or in whole, of the cells of the pancreatic islets of Langerhans. The term “islet cell” includes a glucose-responsive, insulin producing cell derived from a stem cell, e.g., from an induced pluripotent stem cell line.

“Mannitol”, as used herein, refers to D-mannitol unless otherwise explicitly stated.

“Medium molecular weight alginate,” or “MMW-Alg” as used herein means an alginate with an approximate molecular weight of 75 kDa to 150 kDa.

“Mesenchymal stem function cell” or “MSFC,” as those terms are used herein, refers to a cell derived from, or having at least one characteristic specific to a cell of, mesodermal lineage, and wherein the MSFC is i) not in a terminal state of differentiation and ii) can terminally differentiate into one or more cell types. An MSFC does not comprise a cell of endodermal origin, e.g., a gut cell, or of ectodermal origin, e.g., a cell derived from skin, CNS, or a neural cell. In an embodiment, the MSFC is multipotent. In an embodiment, the MSFC is not totipotent. In an embodiment, an MSFC comprises one or more of the following characteristics:
a) it comprises a mesenchymal stem cell (MSC) or a cell derived therefrom, including a cell derived from a primary cell culture of MSCs, a cell isolated directly (without long term culturing, e.g., less than 5 or 10 passages or rounds of cell division since isolation) from naturally occurring MSCs, e.g., from a human or other mammal, a cell derived from a transformed, a pluripotent, an immortalized, or a long term (e.g., more than 5 or 10 passages or rounds of cell division) MSC culture.
b) it comprises a cell that has been obtained from a less differentiated cell, e.g., a cell developed, programmed, or reprogramed (e.g., in vitro) into an MSC or a cell that is, except for any genetic engineering, substantially similar to one or more of a naturally occurring MSC or a cell from a primary or long term culture of MSCs, or a cell described in a) above. Examples of less differentiated cells from which MSFC can be derived include IPS cells, embryonic stem cells, or other totipotent or pluripotent cells; see, e.g., Chen, Y. S. et al (2012) Stem Cells Transl Med 1(83-95); Frobel, J et al (2014) Stem Cell Reports 3(3):414-422; Zou, L et al (2013) Sci Rep 3:2243;
c) it is multipotent, e.g., as measured by any assay capable of providing information about cell multipotency, e.g., microscopy;
d) it exhibits a characteristic mononuclear ovoid, stellate shape or spindle shape, with a round to oval nucleus. The oval elongate nucleus may have prominent nucleoli and a mix of heterochromatin and euchromatin. An MSFC (e.g., an MSC) may have little cytoplasm, but many thin processes that appear to extend from the nucleus;
e) it is capable of cell division, e.g., as measured any assay capable of providing information about cell division, e.g., microscopy. In an embodiment, an MSFC is capable of cell division in culture (e.g., prior to being encapsulated or incorporated into a device). In an embodiment, it is capable of cell division after being encapsulated, e.g., encapsulated as described herein, or incorporated into a device (e.g., a device described herein). In an embodiment, it is incapable of cell division after reaching confluence;
f) it is capable of differentiating into a mesenchymal cell lineage, e.g., an osteoblast, a chrondoblast, an adipocyte, or a fibroblast;
g) it expresses a mesenchymal cell marker, e.g., one, two, three, four, five or all of CD105, CD106, CD73, CD90, Stro-1, CD49a, CD29, CD44, CD146, CD166, TNAP+, THY-1+, Stro-2, Stro-4, and alkaline phosphatase;
h) it does not express significant levels of one, two, three, or any of CD34, CD31, VE-cadherin, CD45, HLA-DR, CD11b and a glycophorin or leukocyte differentiation antigen, e,g, CD14, CD33, CD3 and CD19;
i) it expresses one, two, or all of CD75, CD90, and CD105 and does not express one, two, or any of CD45, CD34, and CD14;
j) it is anti-inflammatory or immune dampening, e.g., as measured by any method capable of providing information regarding inflammation, e.g., in vivo inhibition of T cell proliferation;
k) it is capable of being adherent, e.g., plastic adherent, e.g., as determined by, e.g., visual inspection; or
l) can grow in three dimensions, e.g., as determined by, e.g., visual inspection.

“Parathyroid hormone” or “PTH” as used herein means a polypeptide or peptide that comprises the amino acid sequence of a naturally occurring parathyroid hormone polypeptide or peptide or variant thereof that has a PTH biological activity, e.g., as determined by an art recognized assay. PTH polypeptides and peptides that may be expressed by encapsulated cells described herein include wild-type primate (e.g., human), porcine, canine, and murine proteins, as well as variants of such wild-type proteins. Such PTH polypeptides and peptides may consist essentially of the wild-type human sequence for pre-pro-PTH polypeptide (115 amino acids), pro-PTH polypeptide (90 amino acids), the mature 84-amino acid peptide (PTH(1-84)), and biologically active variants thereof, such as the truncated variant peptide PTH(1-34). PTH peptide variants with one or more amino acid substitutions in the human wild-type sequence have been described, e.g., in U.S. Pat. Nos. 7,410,948 and 8,563,513 and in US Patent Application Publication No. 20130217630. A PTH variant preferably has at least 50%, 75%, 90% or more (including >100%) of a biological activity of the corresponding wild-type PTH. An assay to detect certain PTH variants by tandem mass spectrometry is described in U.S. Pat. No. 8,383,417. A biological activity assay for PTH peptide variants—stimulation of adenylate cyclase as determined by measuring cAMP levels—is described in U.S. Pat. No. 7,410,948.

“Poloxamer”, as used herein, refers to the standard generic term for a class of nonionic triblock linear copolymers composed of a central hydrophobic chain of polyoxypropylene (poly(propylene oxide)) flanked by two polyoxyethylene (poly(ethylene oxide)) moieties. “Poloxamer 188” or “P 188”, as used herein, refers to a poloxamer with an approximate molecular mass of 1800 g/mole for the polyoxypropylene core and an oxyethylene content of about 80% weight percent, e.g., 79.0 to 83.7 percent. In an embodiment, poloxamer 188 has an average molecular weight of 8350 g/mole. In an embodiment, poloxamer 188 has an average molecular weight of 7680 g/mole to 9510 g/mole, e.g., as determined by size exclusion chromatography, and an oxyethylene content of 81.8±1.9% weight percent. In an embodiment, each polyoxyethylene chain in poloxamer 188 has 75-85 (e.g., 80) ethylene oxide monomers and the polyoxypropylene core has 25-30 (e.g., 27) propylene oxide monomers. In an embodiment, poloxamer 188 used in a process described herein substantially meets the specifications set forth in a poloxamer monograph published by the United States Pharmacopeia-National Formulary (USP-NF) or the European Pharmacopoeia (Ph. Eur.) that is official at the time the process is performed.

“Polymer composition”, as used herein, is a composition (e.g., a solution, mixture) comprising one or more polymers. As a class, “polymers’ includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, refers to a polymer comprising amino acid residues linked through peptide bonds and having at least two, and in some embodiments, at least 10, 50, 75, 100, 150 or 200 amino acid residues.

“Prevention,” “prevent,” and “preventing” as used herein refers to a treatment that comprises administering or applying a therapy, e.g., administering a composition of devices encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “replacement therapy” or “replacement protein” is a therapeutic protein or functional fragment thereof that replaces or augments a beneficial function of a protein that is diminished, present in insufficient quantity, altered (e.g., mutated) or lacking in a subject having a disease or condition related to the diminished, altered or lacking protein. Examples are certain blood clotting factors in certain blood clotting disorders or certain lysosomal enzymes in certain lysosomal storage diseases. In an embodiment, a replacement therapy or replacement protein provides the function of an endogenous protein. In an embodiment, a replacement therapy or replacement protein has the same amino acid sequence of a naturally occurring variant of the replaced protein, e.g., a wild type allele or an allele not associated with a disorder. In an embodiment, or replacement therapy or a replacement protein differs in amino acid sequence from a naturally occurring variant, e.g., a wild type allele or an allele not associated with a disorder, e.g., the allele carried by a subject, at no more than about 1, 2, 3, 4, 5, 10, 15 or 20% of the amino acid residues.

“Reference device”, as used herein with respect to a claimed device (e.g., hydrogel capsule), means a device (e.g., hydrogel capsule) that: (i) lacks a particular feature, e.g., FBR-mitigating means (e.g., a barrier compartment comprising an afibrotic compound (as defined herein) or a CBS (as defined herein) (e.g., an RGD polymer), (ii) encapsulates in the cell-containing compartment about the same quantity of cells of the same cell type(s) as in the claimed device, and (iii) has a substantially similar polymer composition and structure as in the claimed device other than lacking the particular feature (e.g., the afibrotic compound or CBS). In an embodiment, the number of live cells in the cell-containing compartment of a reference device is within 80% to 120%, or within 90% to 110%, of the number of live cells in the cell-containing compartment of the claimed device. In an embodiment, the cells in the reference and claimed devices are obtained from the same cell culture. In an embodiment, a substantially similar polymer composition means all polymers in the reference and claimed device, including the polymer component of any CBP-polymer and afibrotic polymer, as applicable, are of the same chemical and molecular weight class (e.g., an alginate with high G content and the same molecular weight range). For example, in an embodiment, the cell-containing compartment of a CBP-null reference device is formed from the unmodified version of the polymer (e.g., alginate) in the CBP-polymer used to form the cell-containing compartment of the claimed device. In some embodiments in which a claimed two-compartment hydrogel millicapsule has (i) an inner compartment formed from a CBP-polymer encapsulating the plurality of cells and (ii) an outer compartment formed from a mixture of a chemically-modified polymer (e.g., a CM-LMW-alginate as described herein) and an unmodified polymer (e.g., an U-HMW-alginate as described herein), then the outer compartments of the reference and claimed capsules are formed from the same polymer mixture, while the inner compartment of the reference capsule is formed from a suspension of cells in the same polymer mixture used for the outer compartment. In an embodiment, a substantially similar structure means the reference and claimed devices have the same number of compartments (e.g., one, two, three, etc.) and about the same size and shape. “RPE cell” as used herein refers to a cell having one or more of the following characteristics: a) it comprises a retinal pigment epithelial cell (RPE) (e.g., cultured using the ARPE-19 cell line (ATCC® CRL-2302™)) or a cell derived therefrom, e.g., by stably transfecting cells cultured from the ARPE-19 cell line with an exogenous sequence that encodes a therapeutic protein or otherwise engineering such cultured ARPE-19 cells to express an exogenous protein or other exogenous substance, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE cell described herein is engineered, e.g., to have a new property, e.g., the cell is engineered to express the therapeutic agent when encapsulated in the polymer composition comprising a CBP or CBS. In other embodiments, an RPE cell is not engineered.

“Saline solution” as used herein, means normal saline, i.e., water containing 0.9% NaCl, unless otherwise specified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, means a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a mouse, a dog, a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Total volume,” as used herein, refers to a volume within one compartment of a multi-compartment device that includes the space occupied by another compartment. For example, the total volume of the second (e.g., outer) compartment of a two-compartment device refers to a volume within the second compartment that includes space occupied by the first compartment. “Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not. “Von Willebrand factor protein” or “VWF protein”, as used herein, means a polypeptide that comprises the amino acid sequence of a naturally occurring VWF polypeptide or variant thereof that has VWF biological activity, e.g., FVIII binding activity, as determined by an art-recognized assay, unless otherwise specified. VWF proteins that may be produced by a device described herein (e.g., expressed by engineered cells contained in the device) include wild-type primate (e.g., human), porcine, canine, and murine proteins, as well as variants of such wild-type proteins. The encapsulated cells may be engineered to encode any of the following VWF polypeptides: precursor VWF of 2813 amino acids, a VWF lacking the signal peptide of 22 amino acids and optionally the prepropeptide of 741 amino acids, mature VWF protein of 2050 amino acids, and truncated variants thereof, such as a VWF fragment sufficient to stabilize endogenous FVIII levels in VWF-deficient mice, e.g, a truncated variant containing the D D3 region (amino acids 764-1247) or the D1D2DID3 region; and VWF variants with one or more amino acid substitutions, e.g., in the D region as described in U.S. Pat. No. 9,458,223. A variant VWF protein preferably has at least 50%, 75%, 90% or more (including >100%) of a biological activity of the corresponding wild-type VWF protein. Art-recognized assays for determining the biological activity of a VWF include ristocetin co-factor activity (Federici A B et al. 2004. Haemaiologica 89:77-85), binding of VWF to GP Iba of the platelet glycoprotein complex Ib-V-IX (Sucker et al. 2006. Cin Appl Thromb Hemost. 12:305-310), and collagen binding (Kallas & Talpsep. 2001. Annals of Hematology 80:466-471).

In some embodiments, the VWF protein produced by a device of the disclosure comprises a naturally-occurring or variant VWF amino acid sequence fused to a heterologous polypeptide or non-polypeptide moiety extending the half-life of the VWF protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts. When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

“Polysaccharide” as used herein, refers to a polymer of monosaccharide or disaccharide carbohydrates bound together by glycosidic linkages. Polysaccharides may be linear or branched. Exemplary monosaccharides include glucose, galactose, mannose, allose, altrose, talose, idose, gulose, fructose, ribose, arabinose, lyxose, xylose, rhamnose, glucuronic acid, galacturonic acid, uronic acid, mannuronic acid, and guluronic acid. Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65.

Devices of the present disclosure may contain a compound of Formula (I) in a prodrug form. Prodrugs are those compounds that readily undergo chemical changes under physiological conditions to provide the compounds useful for preparing devices in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable device, e.g., a particle, a hydrogel capsule. The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element (e.g., a device) as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

The terms “covalent,” “covalent bond,” and “covalent linkage” as used herein refer to a type of chemical bond that involves the sharing of electrons between two neighboring atoms. Examples of covalent bonds include those formed between carbon and hydrogen (C—H bond), carbon atoms (C—C bond), carbon and oxygen atoms (C—O bond), and carbon and nitrogen (C—N bond). Depending on the identity of the atoms, a covalent bond may be a single, double, or triple bond, i.e., a covalent bond may involve the sharing of one, two, or three pairs of electrons.

The terms “ionic,” “ionic bond,” and “ionic linkage” as used herein refer to a type of chemical bond that involved the Coulombic attraction between neighboring atoms (i.e., ions) of opposite charge.

### Modified Polysaccharide Polymers

The polysaccharide polymers described herein are covalently modified with a covalent crosslinker moiety. In an embodiment, the polysaccharide polymer may be linear, branched, or cross-linked polysaccharide polymer, or a polysaccharide polymer of selected molecular weight ranges, degree of polymerization, viscosity or melt flow rate. Branched polysaccharide polymer can include one or more of the following types: star polymers, comb polymers, brush polymers, dendronized polymers, graft-co(polymers), ladders, and dendrimers. A polysaccharide polymer may be a thermoresponsive polymer, e.g., a gel (e.g., becomes a solid or liquid upon exposure to heat or a certain temperature) or a photocrosslinkable polymer. In some embodiments, the polysaccharide polymers may be biodegradable, e.g., contain a labile bond, or may be dissociated by an enzyme, e.g., a lyase. In some embodiments, a polysaccharide polymer is made up of a single type of repeating monomeric unit. In other embodiments, a polysaccharide polymer is made up of different types of repeating monomeric units (e.g., two types of repeating monomeric units, three types of repeating monomeric units, e.g., a polymeric blend). In some embodiments, the polysaccharide may be composed of mannuronic acid and guluronic acid monomers.

In some embodiments, the polymer is a naturally occurring or synthetic polymer. In some embodiments, the polymer is a naturally occurring polysaccharide or a synthetic polysaccharide. In an embodiment, the polysaccharide polymer is a cellulose, e.g., carboxymethyl cellulose. In an embodiment, the polysaccharide polymer is a polylactide, a polyglycoside or a polycaprolactone. In an embodiment, the polysaccharide polymer is a hyaluronate, e.g., sodium hyaluronate. In an embodiment, the polymer is a collagen, elastin or gelatin. In an embodiment, the polymer is chitin.

In some embodiments, the polysaccharide polymer is a hydrogel-forming polymer. Hydrogel-forming polymers comprise a hydrophilic structure that renders them capable of holding large amounts of water in a three-dimensional network. Hydrogel-forming polymers may include polymers which form homopolymeric hydrogels, copolymeric hydrogels, or multipolymer interpenetrating polymeric hydrogels, and may be amorphous, semicrystalline, or crystalline in nature, e.g., as described in Ahmed (2015) J Adv Res 6:105-121. Exemplary hydrogel-forming polymers include proteins (e.g., collagen), gelatin, polysaccharides (e.g., starch, alginate, hyaluronate, agarose), and synthetic polysaccharides.

Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65. A polysaccharide polymer may comprise heparin, chondoitin sulfate, dermatan, dextran, or carboxymethylcellulose. In some embodiments, a polysaccharide polymer is a cross-linked polymer. In some embodiments, a polysaccharide polymer is a cell-surface polysaccharide.

In some embodiments, the polysaccharide polymer is an alginate. Alginate is a polysaccharide made up of β-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M In some embodiments, the ratio of M:G is about 1. In some embodiments, the ratio of M:G is less than 1. In some embodiments, the ratio of M:G is greater than 1. In some embodiments, the alginate has an approximate molecular weight of <75 kDa, and optionally a G:M ratio of ≥1.5. In some embodiments, the alginate has an approximate molecular weight of 75 kDa to 150 kDa and optionally a G:M ratio of ≥1.5. In some embodiments, the alginate has an approximate molecular weight of 150 to 250 kDa and optionally a G:M ratio of ≥1.5.

A polysaccharide polymer (e.g., any of the polymers described herein, for example, any of the alginates described herein) comprising a saccharide moiety having the structure of Formula (I) or a pharmaceutically acceptable salt thereof may be modified on one or more monomeric units. In some embodiments, at least 0.5 percent of the saccharide monomers of a polysaccharide polymer have the structure of Formula (I) (e.g., at least 1, 2.5, 5, 10, 15, 20, 25, 30, 35, 40, 45, 50, 55, 60, 65, 70, 75, 80, 85, 90, 95, 99 percent, or more of the saccharide monomers have the structure of Formula (I). In some embodiments, 0.5 to 50%, 10 to 90%, 10 to 50%, or 25-75%, of the saccharide monomers of a polysaccharide polymer have the structure of Formula (I). In some embodiments, 1 to 20% of the saccharide monomers of a polysaccharide polymer have the structure of Formula (I). In some embodiments, 1 to 10% of the saccharide monomers of a polysaccharide polymer have the structure of Formula (I). In some embodiments, 1 to 50% of the saccharide monomers have the structure of Formula (I).

In some embodiments, the polysaccharide polymer (when comprising a saccharide monomer having the structure of Formula I) comprises an increase in % N (as compared with unmodified polymer) of at least 0.1, 0.2, 0.5, 1.0, 1.5, 2, 2.5, 3, 3.5, 4, 4.5, 5, 5.5, 6, 6.5, 7, 7.5, 8, 8.5, 9, 9.5, or 10% N by weight, where % N is determined by elemental analysis and corresponds to the amount of compound of Formula I in the modified polymer.

In some embodiments, the polysaccharide polymer (when comprising a saccharide monomer having the structure of Formula I) comprises an increase in % N (as compared with unmodified polymer) of 0.1 to 10% N by weight, where % N is determined by elemental analysis and corresponds to the amount of compound of Formula I in the modified polymer.

In some embodiments, the polysaccharide polymer (when comprising a saccharide monomer having the structure of Formula (I) comprises an increase in % N (as compared with unmodified polymer) of 0.1 to 2% N by weight, where % N is determined by elemental analysis and corresponds to the amount of compound of Formula I in the modified polymer.

In some embodiments, the polysaccharide polymer (when comprising a saccharide monomer having the structure of Formula (I) comprises an increase in % N (as compared with unmodified polymer) of 2 to 4% N by weight, where % N is determined by elemental analysis and corresponds to the amount of compound of Formula I in the modified polymer.

In some embodiments, the polysaccharide polymer (when comprising a saccharide monomer having the structure of Formula (I) comprises an increase in % N (as compared with unmodified polymer) of 4 to 8% N by weight, where % N is determined by elemental analysis and corresponds to the amount of compound of Formula I in the modified polymer.

In some embodiments, any of the polysaccharide polymers described herein (e.g., an alginate) comprise a saccharide monomer having one or more of Formula (I-a), (I-b), (I-c), (I-d), (I-e), (I-f), or a pharmaceutically acceptable salt thereof. In some embodiments, the polysaccharide polymer comprises a saccharide monomer having the structure of Formula (II-a). In some embodiments, the polymer is modified with a compound of Formula (II-b). In some embodiments, the polysaccharide polymer comprises a saccharide monomer having the structure of Formula (II-c). In some embodiments, the polysaccharide polymer comprises a saccharide monomer having the structure of Formula (II-d). In some embodiments, the polysaccharide polymer comprises a saccharide monomer having the structure of Formula (II-e). In some embodiments, the polysaccharide polymer comprises a saccharide monomer having the structure of Formula (II-f).

In some embodiments, the polymer (e.g., an alginate) is modified with a compound shown in Table 3.

In some embodiments, a polymer (e.g., an alginate) modified with a compound of Formula (I) is not a modified polymer described in any one of WO2012/112982, WO2012/167223, WO2014/153126, WO2016/187225, WO2016/019391, WO2017/075630, WO 2017/075631, WO 2018/067615, WO 2019/169333, and US 2016-0030359.

### Afibrotic Compounds

In some embodiments, the polymers described herein further comprise at least one afibrotic compound of Formula (I):

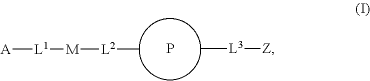

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹; each of L¹ and L³ is independently a bond, alkyl, or heteroalkyl, wherein each alkyl and heteroalkyl is optionally substituted by one or more R²; L² is a bond; M is absent, alkyl, heteroalkyl, cycloalkyl, heterocyclyl, aryl, or heteroaryl, each of which is optionally substituted by one or more R³; P is absent, cycloalkyl, heterocyclyl, or heteroaryl, each of which is optionally substituted by one or more R⁴; Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵; each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶; or R^{C} and R^{D}, taken together with the nitrogen atom to which they are attached, form a ring (e.g., a 5-7 membered ring), optionally substituted with one or more R⁶; each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}) N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R′; each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl; x is 1 or 2; and y is 2, 3, or 4.

In some embodiments, the compound of Formula (I) is a compound of Formula (I-a):

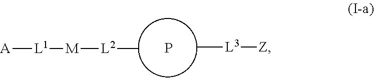

or a pharmaceutically acceptable salt thereof, wherein: A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, N(R^{C})C(O)(C₁-C₆-alkylene)-, —N(R^{C})C(O)(C₁-C₆-alkenylene)-, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹; each of L¹ and L³ is independently a bond, alkyl, or heteroalkyl, wherein each alkyl and heteroalkyl is optionally substituted by one or more R²; L² is a bond; M is absent, alkyl, heteroalkyl, cycloalkyl, heterocyclyl, aryl, or heteroaryl, each of which is optionally substituted by one or more R³; P is heteroaryl optionally substituted by one or more R⁴; Z is alkyl, alkenyl, alkynyl, heteroalkyl, cycloalkyl, heterocyclyl, aryl, or heteroaryl, each of which is optionally substituted by one or more R⁵; each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶; or R^{C} and R^{D}, taken together with the nitrogen atom to which they are attached, form a ring (e.g., a 5-7 membered ring), optionally substituted with one or more R⁶; each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1}), cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷; each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl; x is 1 or 2; and y is 2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂CH₂—)₃. In some embodiments, M is aryl. In some embodiments, M is phenyl. In some embodiments, M is unsubstituted phenyl. In some embodiments, M is

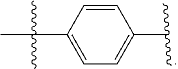 [SMILES: *c1ccc(*)cc1]

In some embodiments, M is phenyl substituted with R⁷ (e.g., 1 R⁷). In some embodiments, M is

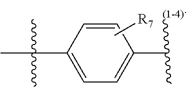

In some embodiments, R⁷ is CF₃.

In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is

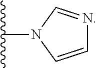 [SMILES: *n1ccnc1]

In some embodiments, P is triazolyl. In some embodiments, P is 1,2,3-triazolyl. In some embodiments, P is

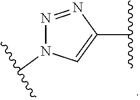 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

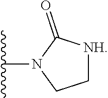 [SMILES: *N1CCNC1=O]

In some embodiments, P is thiomorpholinyl-1,1-dioxidyl. In some embodiments, P is

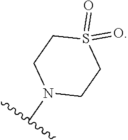 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

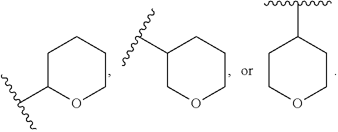

In some embodiments, Z is a 4-membered oxygen-containing heterocyclyl. In some embodiments, Z is

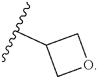 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

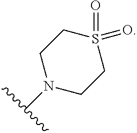 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

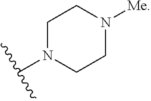 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

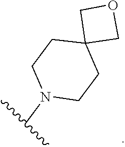 [SMILES: *N1CCC2(CC1)COC2]

In some embodiments, Z is 1-oxa-3,8-diazaspiro[4.5]decan-2-one. In some embodiments, Z is

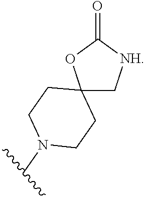 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1} or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

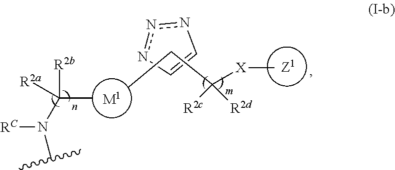

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

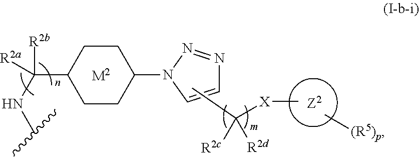

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

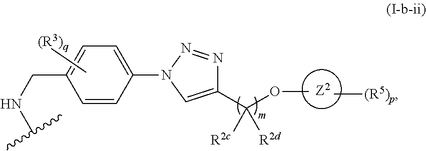

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

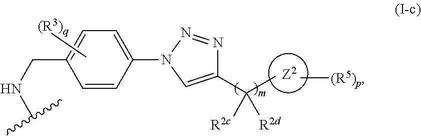

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

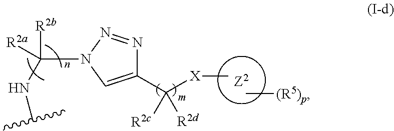

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

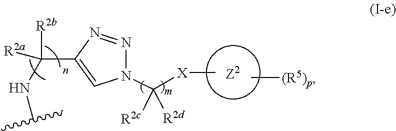

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

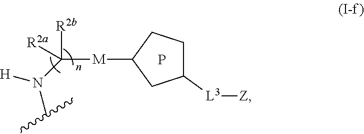

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

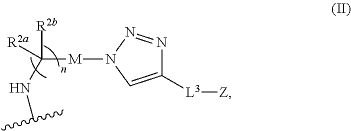

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

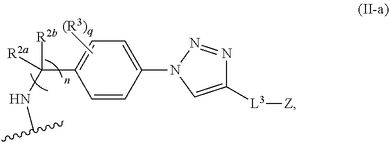

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

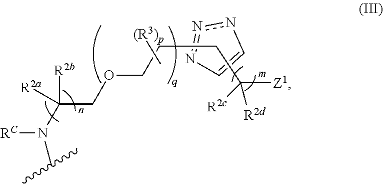

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

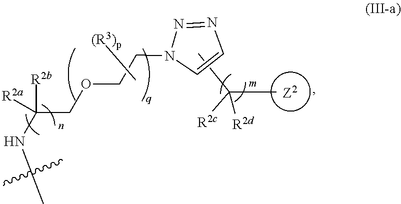

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

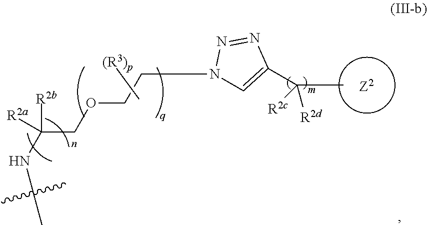

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

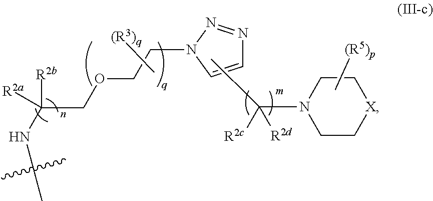

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

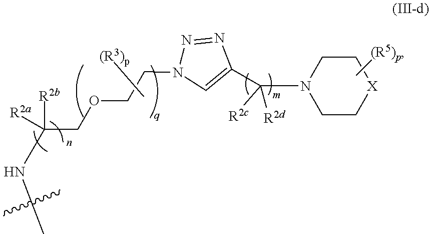

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (I) is a compound of Formula (III-e):

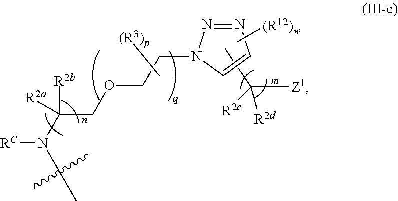

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵ each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (I) is a compound of Formula (III-f):

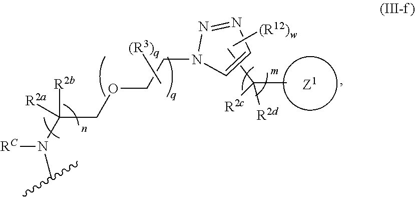

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (I) is a compound of Formula (III-g):

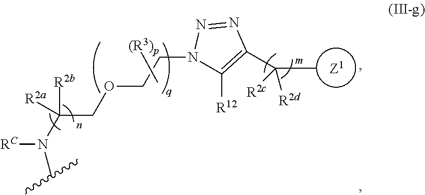

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (I) is a compound of Formula (III-h):

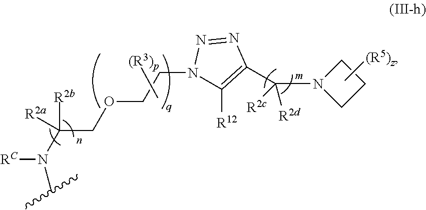

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (I) is a compound of Formula (III-i):

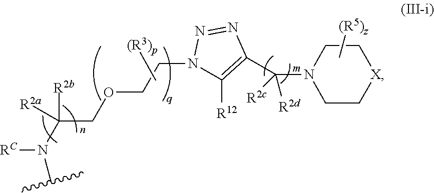

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide).

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl).

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is 0, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl).

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl).

In some embodiments, the compound is a compound of Formula (I-c). In some embodiments of Formula (I-c), each of R²⁰ and R^{2d} is independently hydrogen, m is 1, p is 1, q is 0, R⁵ is —CH₃, and Z is heterocyclyl (e.g., a nitrogen-containing heterocyclyl, e.g., piperazinyl).

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is 0, p is 0, and Z is heterocyclyl (e.g., an oxygen-containing heterocyclyl, e.g., tetrahydropyranyl, tetrahydrofuranyl, oxetanyl, or oxiranyl).

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃

In some embodiments of Formula (II-a), each of R^{2a} and R^{2b} is independently hydrogen, n is 1, L³ is a bond or —CH₂, and Z is hydrogen or —OH.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃).

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂).

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl).

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 3, or a pharmaceutically acceptable salt thereof. In some embodiments, the exterior surface and/or one or more compartments within a device described herein comprises a small molecule compound shown in Table 3, or a pharmaceutically acceptable salt thereof.

TABLE 3
Exemplary Compounds of Formula (I)

| Compound | |
|---|---|
| No. | Structure |
| 100 | 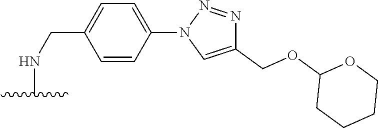 [SMILES: *NCc1ccc(-n2cc(COC3CCCCO3)nn2)cc1] |
| 101 | 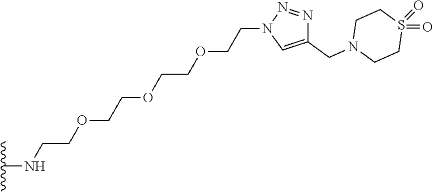 [SMILES: *NCCOCCOCCOCCn1cc(CN2CCS(=O)(=O)CC2)nn1] |
| 102 | 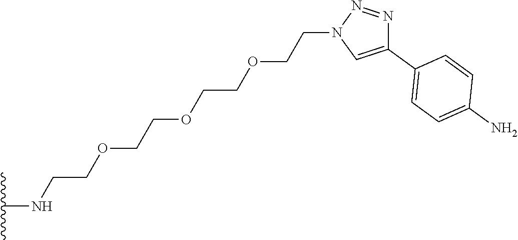 [SMILES: *NCCOCCOCCOCCn1cc(-c2ccc(N)cc2)nn1] |
| 103 | 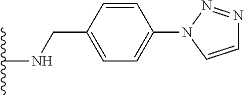 [SMILES: *NCc1ccc(-n2ccnn2)cc1] |
| 104 | 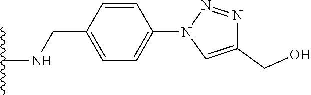 [SMILES: *NCc1ccc(-n2cc(CO)nn2)cc1] |
| 105 | 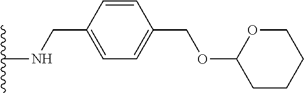 [SMILES: *NCc1ccc(COC2CCCCO2)cc1] |
| 106 | 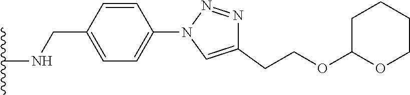 [SMILES: *NCc1ccc(-n2cc(CCOC3CCCCO3)nn2)cc1] |
| 107 | 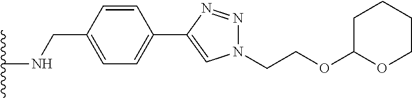 [SMILES: *NCc1ccc(-c2cn(CCOC3CCCCO3)nn2)cc1] |
| 108 | 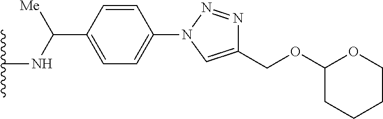 [SMILES: *NC(C)c1ccc(-n2cc(COC3CCCCO3)nn2)cc1] |
| 109 | 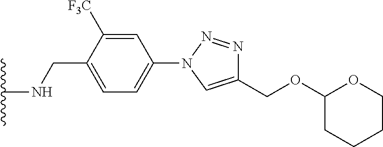 [SMILES: *NCc1ccc(-n2cc(COC3CCCCO3)nn2)cc1C(F)(F)F] |
| 110 | 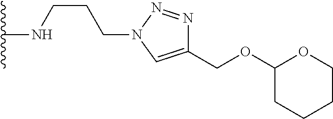 [SMILES: *NCCCn1cc(COC2CCCCO2)nn1] |
| 111 | 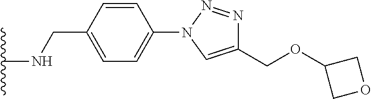 [SMILES: *NCc1ccc(-n2cc(COC3COC3)nn2)cc1] |
| 112 | 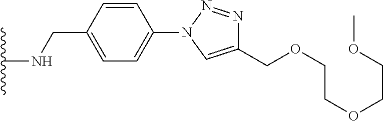 [SMILES: *NCc1ccc(-n2cc(COCCOCCOC)nn2)cc1] |
| 113 | 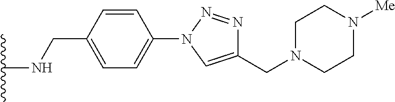 [SMILES: *NCc1ccc(-n2cc(CN3CCN(C)CC3)nn2)cc1] |
| 114 | 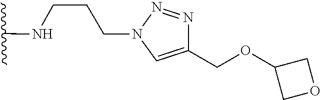 [SMILES: *NCCCn1cc(COC2COC2)nn1] |
| 115 | 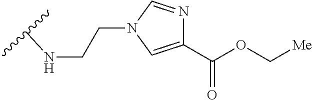 [SMILES: *NCCn1cnc(C(=O)OCC)c1] |
| 116 | 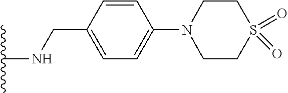 [SMILES: *NCc1ccc(N2CCS(=O)(=O)CC2)cc1] |
| 117 | 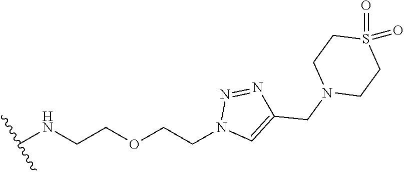 [SMILES: *NCCOCCn1cc(CN2CCS(=O)(=O)CC2)nn1] |
| 118 | 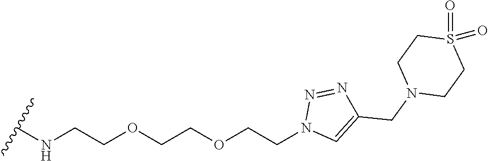 [SMILES: *NCCOCCOCCn1cc(CN2CCS(=O)(=O)CC2)nn1] |
| 119 | 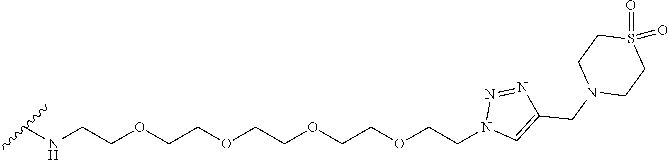 [SMILES: *NCCOCCOCCOCCOCCn1cc(CN2CCS(=O)(=O)CC2)nn1] |
| 120 | 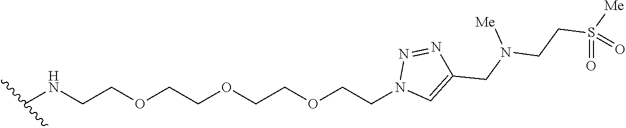 [SMILES: *NCCOCCOCCOCCn1cc(CN(C)CCS(C)(=O)=O)nn1] |
| 121 | 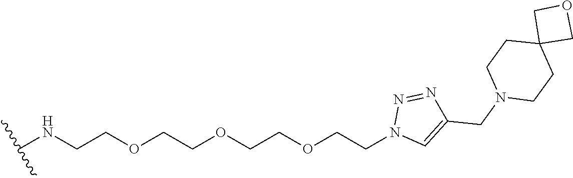 [SMILES: *NCCOCCOCCOCCn1cc(CN2CCC3(CC2)COC3)nn1] |
| 122 | 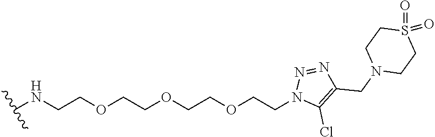 [SMILES: *NCCOCCOCCOCCn1nnc(CN2CCS(=O)(=O)CC2)c1Cl] |
| 123 | 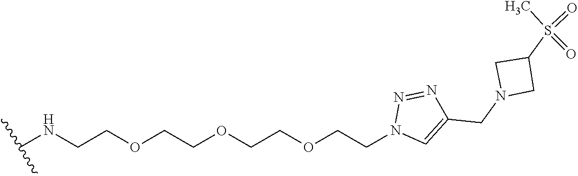 [SMILES: *NCCOCCOCCOCCn1cc(CN2CC(S(C)(=O)=O)C2)nn1] |
| 125 | 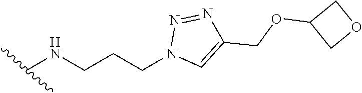 [SMILES: *NCCCn1cc(COC2COC2)nn1] |
| 126 | 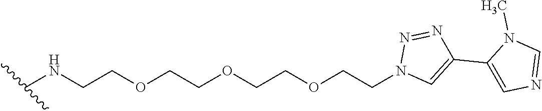 [SMILES: *NCCOCCOCCOCCn1cc(-c2cncn2C)nn1] |
| 128 | 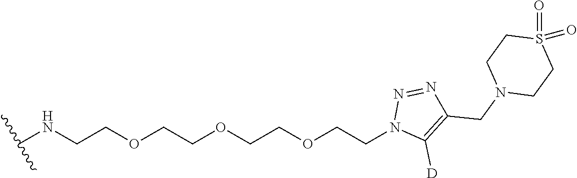 [SMILES: *NCCOCCOCCOCCn1nnc(CN2CCS(=O)(=O)CC2)c1[2H]] |
| 129 | 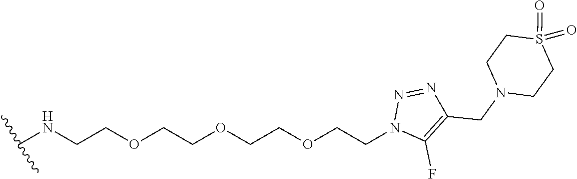 [SMILES: *NCCOCCOCCOCCn1nnc(CN2CCS(=O)(=O)CC2)c1F] |
| 130 | 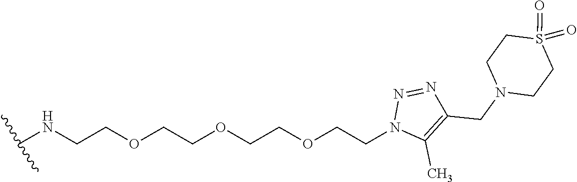 [SMILES: *NCCOCCOCCOCCn1nnc(CN2CCS(=O)(=O)CC2)c1C] |
| 131 | 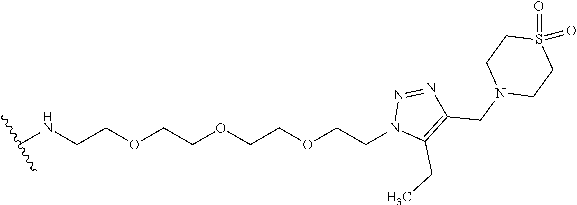 [SMILES: *NCCOCCOCCOCCn1nnc(CN2CCS(=O)(=O)CC2)c1CC] |
| 132 | 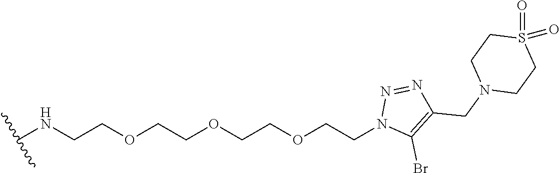 [SMILES: *NCCOCCOCCOCCn1nnc(CN2CCS(=O)(=O)CC2)c1Br] |
| 133 | 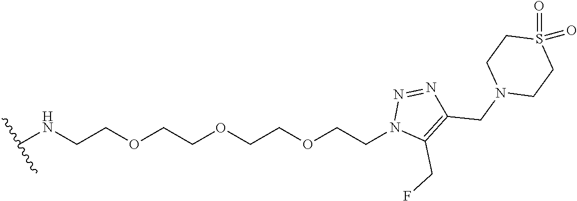 [SMILES: *NCCOCCOCCOCCn1nnc(CN2CCS(=O)(=O)CC2)c1CF] |
| 134 | 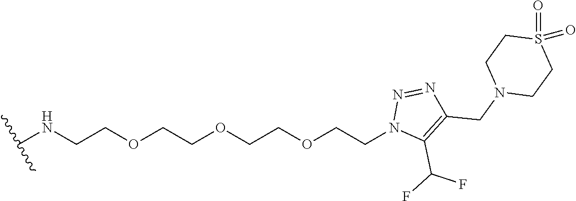 [SMILES: *NCCOCCOCCOCCn1nnc(CN2CCS(=O)(=O)CC2)c1C(F)F] |
| 135 | 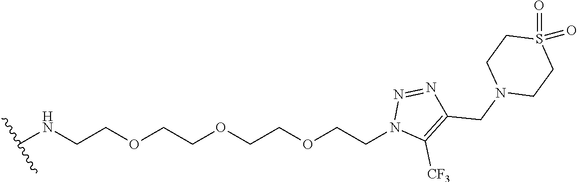 [SMILES: *NCCOCCOCCOCCn1nnc(CN2CCS(=O)(=O)CC2)c1C(F)(F)F] |
| 136 | 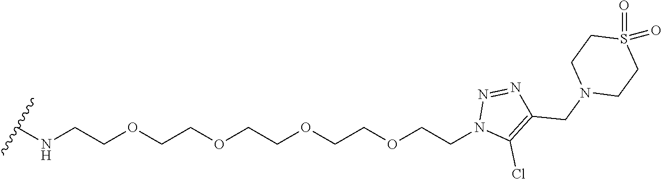 [SMILES: *NCCOCCOCCOCCOCCn1nnc(CN2CCS(=O)(=O)CC2)c1Cl] |
| 137 | 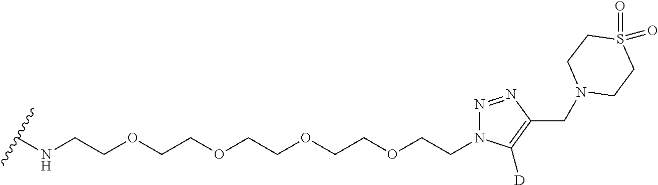 [SMILES: *NCCOCCOCCOCCOCCn1nnc(CN2CCS(=O)(=O)CC2)c1[2H]] |
| 138 | 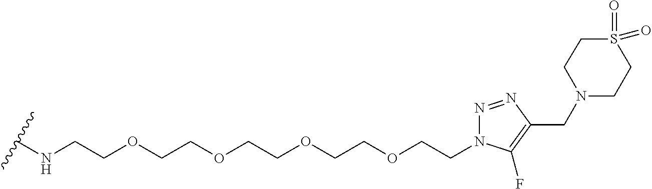 [SMILES: *NCCOCCOCCOCCOCCn1nnc(CN2CCS(=O)(=O)CC2)c1F] |
| 139 | 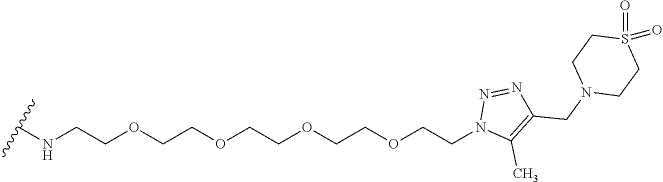 [SMILES: *NCCOCCOCCOCCOCCn1nnc(CN2CCS(=O)(=O)CC2)c1C] |
| 140 | 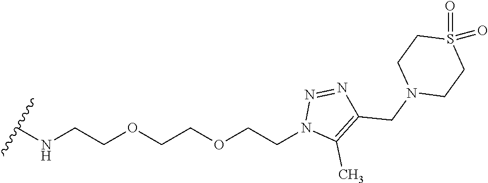 [SMILES: *NCCOCCOCCn1nnc(CN2CCS(=O)(=O)CC2)c1C] |
| 141 | 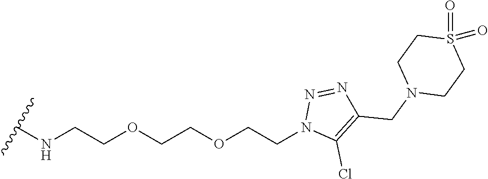 [SMILES: *NCCOCCOCCn1nnc(CN2CCS(=O)(=O)CC2)c1Cl] |
| 142 | 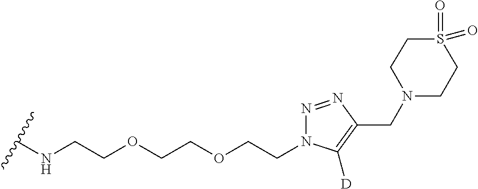 [SMILES: *NCCOCCOCCn1nnc(CN2CCS(=O)(=O)CC2)c1[2H]] |
| 143 | 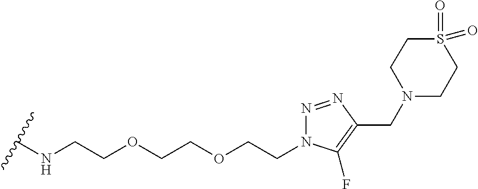 [SMILES: *NCCOCCOCCn1nnc(CN2CCS(=O)(=O)CC2)c1F] |
| 144 | 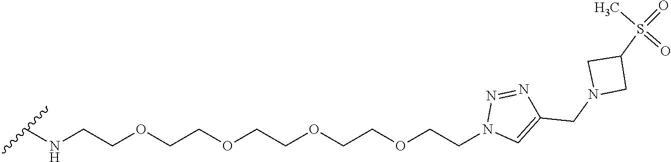 [SMILES: *NCCOCCOCCOCCOCCn1cc(CN2CC(S(C)(=O)=O)C2)nn1] |
| 145 | 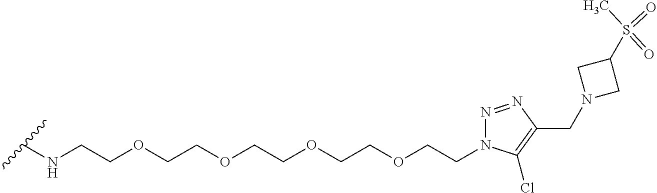 [SMILES: *NCCOCCOCCOCCOCCn1nnc(CN2CC(S(C)(=O)=O)C2)c1Cl] |
| 146 | 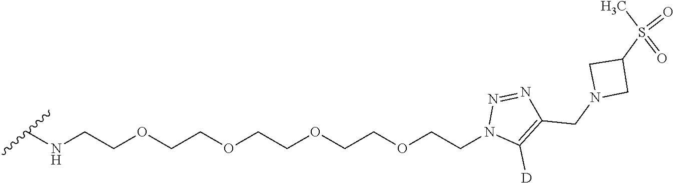 [SMILES: *NCCOCCOCCOCCOCCn1nnc(CN2CC(S(C)(=O)=O)C2)c1[2H]] |
| 147 | 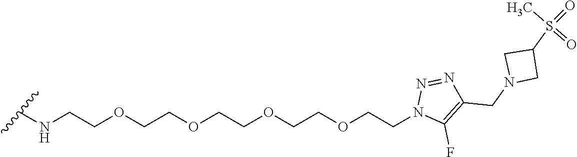 [SMILES: *NCCOCCOCCOCCOCCn1nnc(CN2CC(S(C)(=O)=O)C2)c1F] |
| 148 | 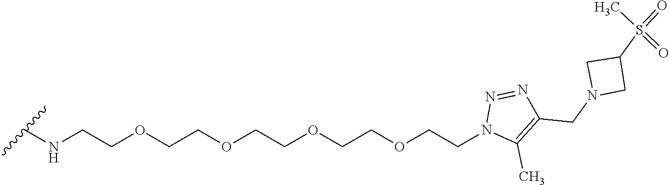 [SMILES: *NCCOCCOCCOCCOCCn1nnc(CN2CC(S(C)(=O)=O)C2)c1C] |
| 149 | 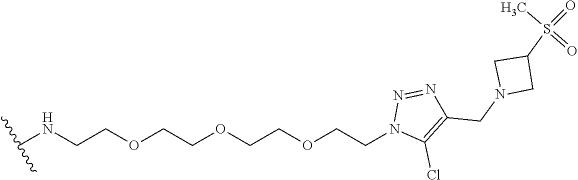 [SMILES: *NCCOCCOCCOCCn1nnc(CN2CC(S(C)(=O)=O)C2)c1Cl] |
| 150 | 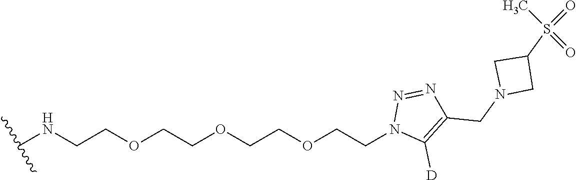 [SMILES: *NCCOCCOCCOCCn1nnc(CN2CC(S(C)(=O)=O)C2)c1[2H]] |
| 151 | 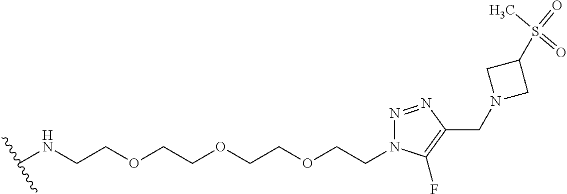 [SMILES: *NCCOCCOCCOCCn1nnc(CN2CC(S(C)(=O)=O)C2)c1F] |
| 152 | 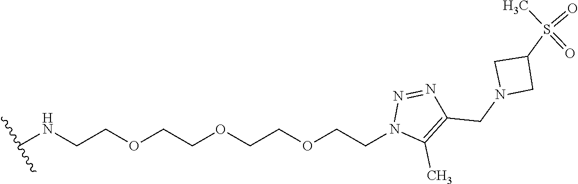 [SMILES: *NCCOCCOCCOCCn1nnc(CN2CC(S(C)(=O)=O)C2)c1C] |
| 153 | 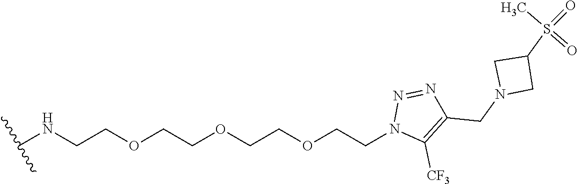 [SMILES: *NCCOCCOCCOCCn1nnc(CN2CC(S(C)(=O)=O)C2)c1C(F)(F)F] |
| 154 | 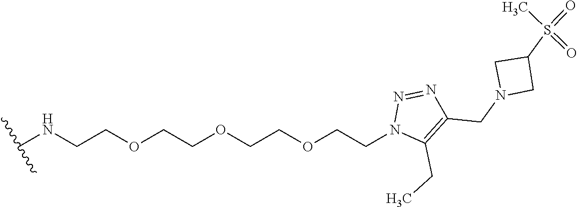 [SMILES: *NCCOCCOCCOCCn1nnc(CN2CC(S(C)(=O)=O)C2)c1CC] |

Conjugation of any of the compounds in Table 3 to a polymer (e.g., an alginate) may be performed as described in Example 2 of WO 2019/195055 or any other suitable chemical reaction.

In some embodiments, the compound is a compound of Formula (I) (e.g., Formulas (I-a), (I-b), (I-c), (I-d), (I-e), (I-f), (II), (II-a), (III), (III-a), (III-b), (III-c), (III-d), (III-e), (III-f), (III-g), (III-h), or (III-i)), or a pharmaceutically acceptable salt thereof and is selected from:

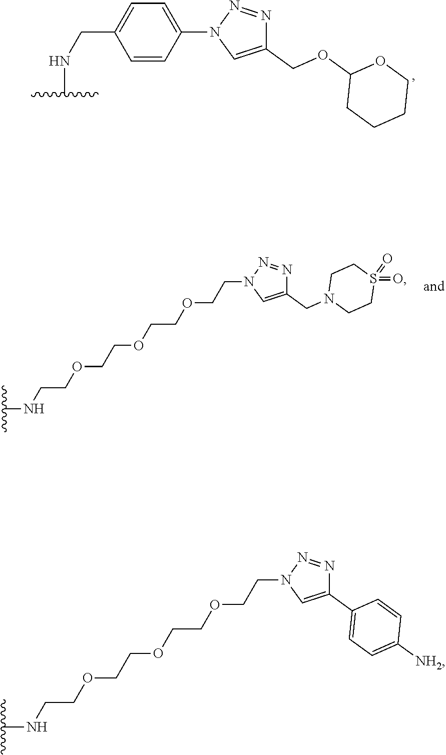 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the polysaccharide polymer or device (e.g., hydrogel capsule) described herein comprises the compound of

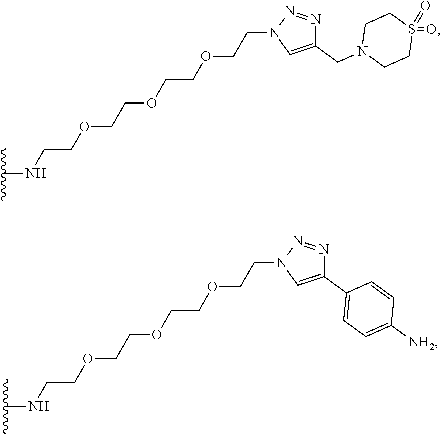 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

In some embodiments, a compound of Formula (I) (e.g., Compound 101 in Table 3) is covalently attached to an alginate (e.g., an alginate with approximate MW<75 kDa, G:M ratio ≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429.

### Crosslinking Moieties

In some embodiments, the crosslinking moiety can undergo a thiol-ene click reaction. In some embodiments, the crosslinking moiety comprises a thiol. In some embodiments, the thiol comprises an alkyl thiol or aryl thiol. In some embodiments, the click crosslinker may contain more than one thiol group. In some embodiments, the click crosslinker may two, three, four, five or six thiol groups, in some embodiments, the thiol is a compound of Formula (IV):

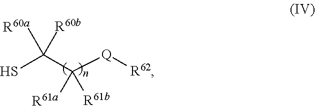

or a pharmaceutically acceptable salt or tautomer thereof, wherein Q is O, NR³³, or C(R^{34a})(R^{34b}); each of R³³, R^{34a}, R^{34b}, R^{60a}, R^{60b}, R^{61a}, R^{61b}, and R⁶² is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the compound of Formula (IV) is a compound of Formula (IV-a):

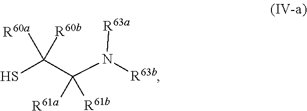

or a pharmaceutically acceptable salt or tautomer thereof, wherein each of R^{60a}, R^{60b}, R^{61a}, R^{61b}, R^{63a}, and R^{63b}, is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1} and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the compound of Formula (IV) is a compound of Formula (IV-b):

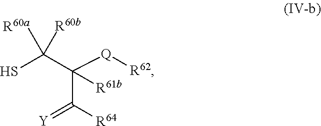

or a pharmaceutically acceptable salt or tautomer thereof, wherein each of Q and Y is O, NR³³, or C(R^{34a})(R^{34b}); each of R³³, R^{34a}, R^{34b}, R^{60a}, R^{60b}, R^{61b}, R⁶² and R⁶⁴ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In sone embodiments, the compound of Formula (IV) is a compound of Formula (IV-c):

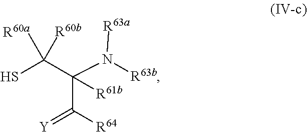

or a pharmaceutically acceptable salt or tautomer thereof, wherein Y is, O, NR³³, or C(R^{34a})(R^{34b}); each of R³³, R^{34a}, R^{34b}, R^{60a}, R^{60b}, R^{61b}, R^{63a}, R^{63b}, and R⁶⁴ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker moiety comprises an alkenyl group. In some embodiments, the crosslinker comprises a cyclyl or heterocyclyl group. In some embodiments, the crosslinker comprises a norbornenyl moiety. In some embodiments, the crosslinker is a compound of Formula (V):

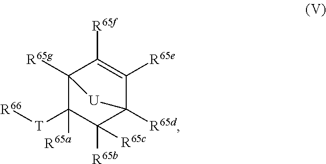

or a pharmaceutically acceptable salt or tautomer thereof, wherein each of T and U is independently O, NR³³, or C(R^{34a})(R^{34b}); each of R³³, R^{34a}, R^{34b}, R^{65a}, R^{65b}, R^{65c}, R^{65d}, R^{65e}, R^{65f}, R^{65g} and R⁶⁶ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (V) is a compound of Formula (V-a):

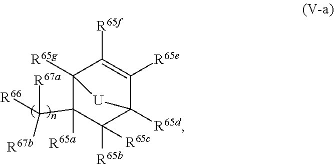

or a pharmaceutically acceptable salt or tautomer thereof, wherein U is O, NR³³, or C(R^{34a})(R^{34b}); each of R³³, R^{34a}, R^{34b}, R^{65a}, R^{65b}, R^{65c}, R^{65d}, R^{65e}, R^{65f}, R⁶⁵, R⁶⁶, R^{67a}, R^{67b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (V) is a compound of Formula (V-b):

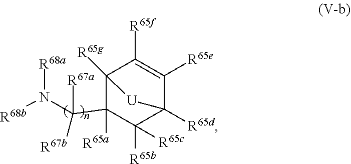

or a pharmaceutically acceptable salt or tautomer thereof, wherein U is O, NR³³, or C(R^{34a})(R^{34b}); each of R³³, R^{34a}, R^{34b}, R^{65a}, R^{65b}, R^{65c}, R^{65d}, R^{65e}, R^{65f}, R⁶⁵, R⁶⁶, R^{67a}, R^{67b}, R^{68a}, and R^{68b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the click crosslinker is a compound of Formula (VI):

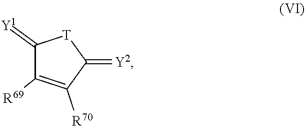

or a pharmaceutically acceptable salt or tautomer thereof, wherein each of T, Y¹, and Y² is independently O, NR³³, or C(R^{34a})(R^{34b}); each of R³³, R^{34a}, R^{34b}, R⁶⁹, and R⁷⁰ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (VI) is a compound of Formula (VI-a):

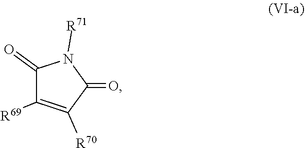

or a pharmaceutically acceptable salt or tautomer thereof, wherein each of R⁶⁹, R⁷⁰, and R⁷¹ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (VI) is a compound of Formula (VI-b):

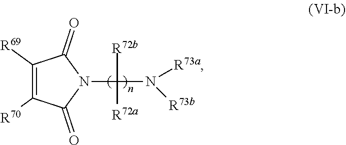

or a pharmaceutically acceptable salt or tautomer thereof, wherein each of R⁶⁹, R⁷⁰, R^{72a}, R^{72b}, R^{73a}, and R^{73b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker comprises a maleimide.

In some embodiments, the crosslinker is comprises an aryl or heteroaryl group. In some embodiments, the click crosslinker is a compound of Formula (VII):

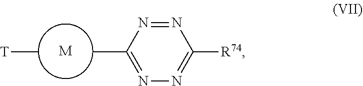

or a pharmaceutically acceptable salt or tautomer thereof, wherein T is O, NR³³, or C(R^{34a})(R^{34b}); Ring M is cycloalkyl, heterocyclyl, aryl, heteroaryl, each of which is optionally substituted with 1-6 R⁷; each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (VII) is a compound of Formula (VII-a):

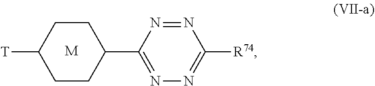

or a pharmaceutically acceptable salt or tautomer thereof, wherein T is O, NR³³, or C(R^{34a})(R^{34b}); Ring M is cycloalkyl, heterocyclyl, aryl, heteroaryl, each of which is optionally substituted with 1-6 R⁷; each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (VII) is a compound of Formula (VII-b):

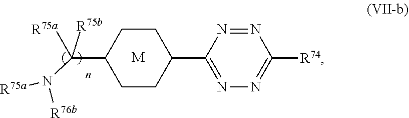

or a pharmaceutically acceptable salt or tautomer thereof, wherein Ring M is cycloalkyl, heterocyclyl, aryl, heteroaryl, each of which is optionally substituted with 1-6 R⁷; each of R⁷⁴, R^{75a}, R^{75b}, R^{76a}, and R^{76b} is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker of Formula (VII) is a compound of Formula (VII-c):

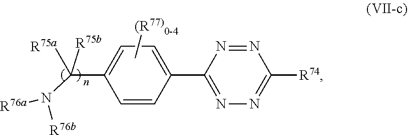

or a pharmaceutically acceptable salt or tautomer thereof, wherein each of R⁷⁴, R^{75a}, R^{75b}, R^{76a}, R^{76b}, and R⁷⁷ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl.

In some embodiments, the crosslinker moiety comprises a tetrazinyl moiety.

In some embodiments, the compound of any one of Formulas (IV), (V), (VI), and (VII) is selected from a compound in Table 4:

TABLE 4
Exemplary crosslinking moieties

| Compound No. | Structure |
|---|---|
| 200 | 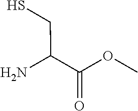 |
| 201 | 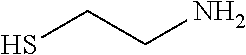 |
| 202 | 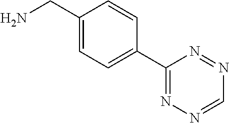 [SMILES: NCc1ccc(-c2nncnn2)cc1] |
| 203 | 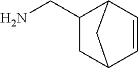 |
| 204 | 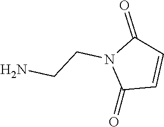 |

Polymers Modified with a Crosslinking Agent

The crosslinking agent may be covalently bound to a polysaccharide, e.g., an alginate. The modified polysaccharide polymer, e.g., modified alginate polymer, may be capable of being crosslinked to another polymer. In an embodiment, the polysaccharide polymer is modified with more than one type of crosslinking agent.

In an embodiment, the modified polysaccharide is a compound of Formula (VIII):

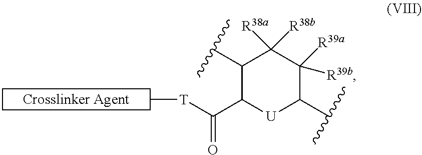

or a pharmaceutically acceptable salt or tautomer thereof, wherein each of T and U is independently C(R⁴⁰)(R⁴¹), O, or N(R⁴²); each of R^{38a}, R^{38b}, R^{39a}, R^{39b}, R⁴⁰, R⁴¹, and R⁴² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R³² and R³⁵ is hydrogen, alkyl, heteroalkyl, halo, cyano, nitro, amino, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; and the click crosslinker has the structure of Formula (IV), (IV-a), (IV-b), (IV-c), (V), (V-a), (V-b), (VI), (VI-a), (VI-b), (VII), (VII-a), (VII-b) and (VII-c).
Polysaccharide Polymers Modified with Click Crosslinkers and Afibrotic Small Molecule Compounds

In an embodiment, the polysaccharide polymer comprises the structure of Formula (IX):

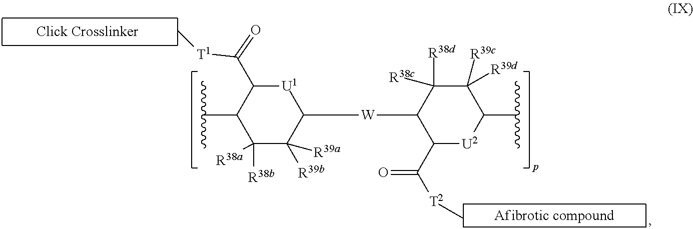

or a pharmaceutically acceptable salt or tautomer thereof, wherein each of W, T¹, T², U¹, and U² is independently C(R⁴⁰)(R⁴¹), O, or N(R⁴²); each of R^{38a}, R^{38b}, R^{38c}, R^{38d} R^{39a}, R^{39b}, R^{39a}, R^{39b}, R⁴⁰, R⁴¹, and R⁴² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and each R⁷ is independently alkyl, alkenyl, alkynyl, heteroalkyl, halogen, cyano, oxo, hydroxyl, cycloalkyl, or heterocyclyl; p is an integer from 1-100; the afibrotic compound has the structure of Formulas (I), (I-a), (I-b), (I-b-i), (I-b-ii), (I-c), (I-d), (I-e), (I-f), (II), (II-a), (III), (III-a), (III-b), (III-c), (III-d), (III-e), (III-f), (III-g), (III-h) or (III-i); and the click crosslinker has the structure of Formula (IV), (IV-a), (IV-b), (IV-c), (V), (V-a), (V-b), (VI), (VI-a), (VI-b), (VII), (VII-a), (VII-b) or (VII-c).

In an embodiment, the modified polysaccharide polymer is a compound selected from Table 5.

TABLE 5
Exemplary Modified Polysaccharide Polymers of Formulas (VIII and IX)

| Compound No. | Structure |
|---|---|
| 300 | 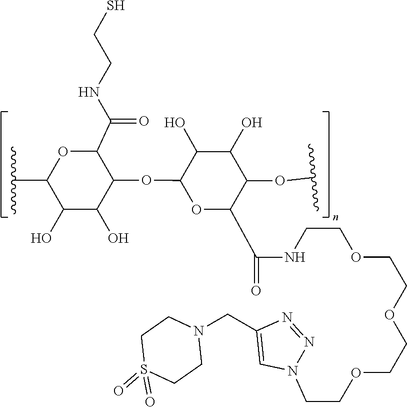 |
| 301 | 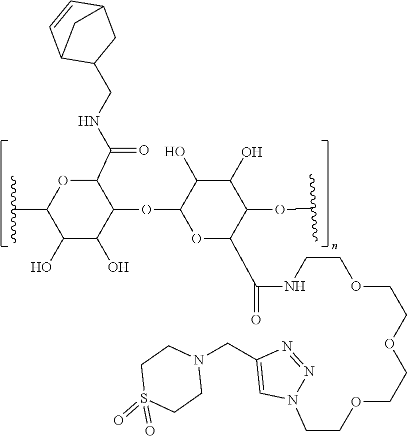 |
| 302 | 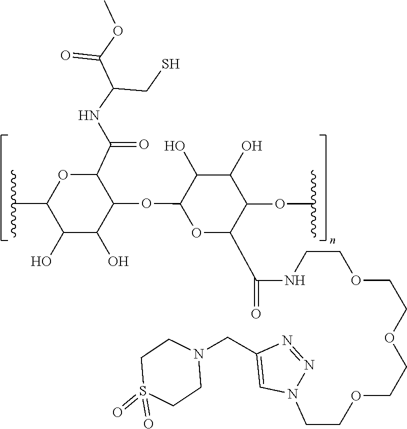 |
| 303 | 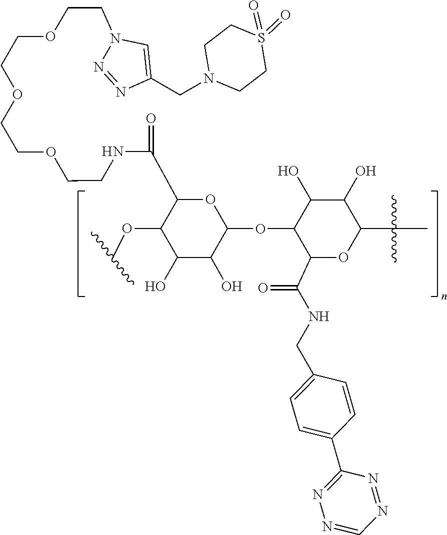 |
| 304 | 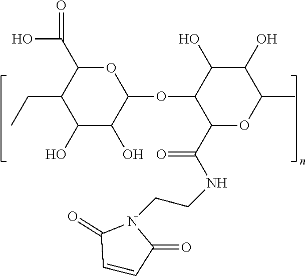 |
| 305 | 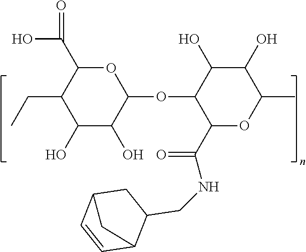 |
| 306 | 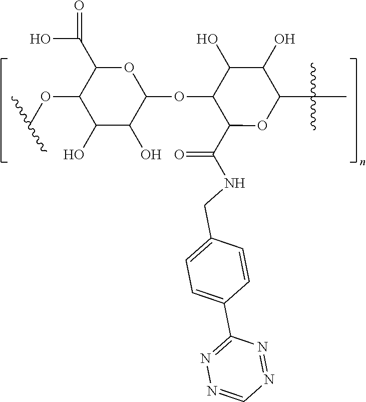 |
| 307 | 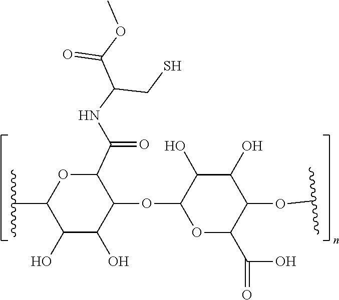 |
| 308 | 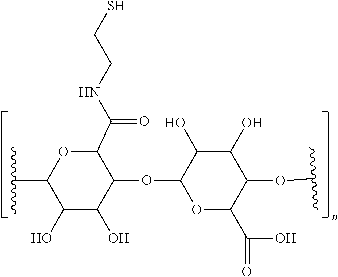 |
| 309 | 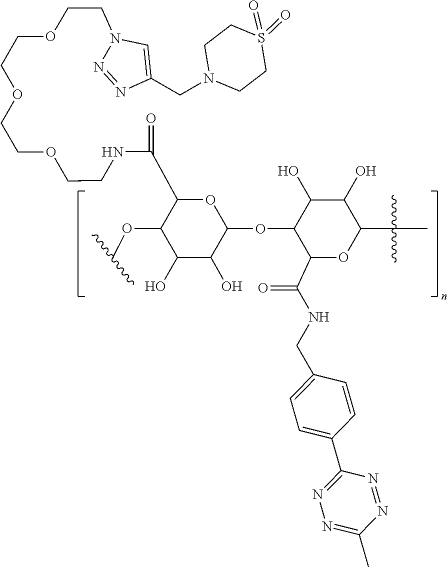 |

or a pharmaceutically acceptable salt thereof.
The polysaccharide polymers described herein may be modified on any suitable functional group (e.g., carboxyl or hydroxyl group). In an embodiment, the polysaccharide polymers are modified on a single type of functional group. In an embodiment, the polysaccharide polymers are modified on more than one type of functional group. In an embodiment, the polysaccharide polymers described herein may be modified on one or more functional groups by a compound of Formula (I) and/or a compound of Formulae (IV)—(VII).

In an embodiment, the degree of modification (i.e., percent of functional groups of the polymer modified with a photoactive crosslinker) is greater than 99%. In an embodiment, the degree of modification of the polymer is less than 99%. In an embodiment, the degree of modification is greater than about 95%. In an embodiment, the degree of modification is about 50%. In an embodiment, the polysaccharide polymers have a degree of modification between about 10% to about 99%. In an embodiment, the degree of modification is between about 10% to about 80%. In an embodiment, the degree of modification between is between about 1% to about 75%. In an embodiment, the polysaccharide polymers have a degree of modification between about 1% to about 70%. In an embodiment, the polysaccharide polymers have a degree of modification between about 1% to about 65%. In an embodiment, the polysaccharide polymers have a degree of modification between about 1% to about 60%. In an embodiment, the polysaccharide polymers have a degree of modification between about 1% to about 55%. In an embodiment, the polysaccharide polymers have a degree of modification between about 1% to about 50%. In an embodiment, the polysaccharide polymers have a degree of modification between about 1% to about 45%. In a preferred embodiment, not all functional groups (e.g. carboxyl groups) of the modified polysaccharide are substituted, which allows for both ionic and covalent crosslinking.

In an embodiment, the polysaccharide polymers described herein are modified with one, two, three, or more unique compounds. In an embodiment, the polysaccharide polymers described herein are modified with a photoactive crosslinker (e.g. a compound of Formula (IV), a compound of Formula (I), and a cell adhesion molecule (e.g., RGD). In an embodiment, the polysaccharide polymers described herein are modified with a photoactive crosslinker. In an embodiment, the polysaccharide polymers described herein are modified with compound of Formula (I). In an embodiment, the polysaccharide polymers described herein are modified with a cell adhesion molecule. In an embodiment, the polysaccharide polymers described herein are modified with both a photoactive crosslinker and a cell adhesion molecule. In a preferred embodiment, the polysaccharide polymers described herein are modified with both a photoactive crosslinker and a compound of Formula (I). In an embodiment, the polysaccharide polymers described herein are modified with both a cell adhesion molecule and a compound of Formula (I).

In an embodiment, the degree of modification (i.e., percent of functional groups of the polymer modified with a clickable crosslinker) is greater than 99%. In an embodiment, the degree of modification of the polymer is less than 99%. In an embodiment, the degree of modification is greater than about 95%. In an embodiment, the degree of modification is about 50%. In an embodiment, the polysaccharide polymers have a degree of modification between about 1% to about 99%. In an embodiment, the degree of modification is between about 1% to about 80%. In an embodiment, the degree of modification between is between about 1% to about 75%. In an embodiment, the polysaccharide polymers have a degree of modification between about 1% to about 70%. In an embodiment, the polysaccharide polymers have a degree of modification between about 1% to about 65%. In an embodiment, the polysaccharide polymers have a degree of modification between about 1% to about 60%. In an embodiment, the polysaccharide polymers have a degree of modification between about 1% to about 55%. In an embodiment, the polysaccharide polymers have a degree of modification between about 1% to about 50%. In an embodiment, the polysaccharide polymers have a degree of modification between about 1% to about 45%. In a preferred embodiment, not all functional groups (e.g. carboxyl groups) of the modified polysaccharide are substituted, which allows for both ionic and covalent crosslinking.

In an embodiment, the degree of modification (i.e., percent of functional groups of the polymer modified with a clickable crosslinker) is about 1%, 2%, 3%, 4%, 5%, 6%, 7%, 8%, 9%, 10%, 15%, 20%, 25%, 35%, 40%, 45%, 50%, 55%, 60%, 65%, 70%, 75%, 80%, 85%, 90%, 95% or 99%. In an embodiment, the degree of modification (i.e., percent of functional groups of the polymer modified with a clickable crosslinker) is greater than about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99%. In an embodiment, the degree of modification (i.e., percent of functional groups of the polymer modified with a clickable crosslinker) is less than about 1%, 2%, 3%, 4%, %, 6%, 7%, 8%, 9%, 10%, 15%, 20%, 25%0, 30%, 35%, 40%, 45%, 50%, 55%, 60%, 65%, 70%, 75%, 80%, 85%, 90%, 95% or 99%.

In some embodiments, the polysaccharide polymers described herein retain sufficient unreacted carboxylic acid groups to allow for ionic crosslinking, e.g., when the polymer is used to prepare hydrogel capsules with dual cross-linking. In some embodiments, the polysaccharide polymers described herein do not comprise a degree of modification of more than 10% of the carboxylic acid groups. In some embodiments, the polysaccharide polymers described herein do not comprise a degree of modification of more than 5% of the carboxylic acid groups. In some embodiments, the polysaccharide polymers described herein do not comprise a degree of modification of more than 5%, 6%, 7%, 8%, 9%, or 10% of the carboxylic acid groups.

### Features of Hydrogel Capsules

The present disclosure further features hydrogel capsules comprising the polysaccharide polymers described herein. The hydrogel capsules may be produced by crosslinking the crosslinking groups (i.e., covalent crosslinking) or by ionically crosslinking, e.g., in the presence of a divalent cation (e.g., Ba2+). In an embodiment, a hydrogel capsule described herein is produced by reacting the crosslinkers. In an embodiment, a hydrogel capsule described herein is produced by covalently crosslinking and ionic crosslinking. A person skilled in the art will recognize that other methods of initiating polymerization are possible including thermal, ultrasonic, and gamma radiation in the presence of appropriate initiators.

The hydrogel capsules described herein are formed by the crosslinking of one or more types of polysaccharide polymers. In an embodiment, the hydrogel capsule comprises only polysaccharide polymers. In an embodiment, the hydrogel capsule comprises polysaccharide polymers of the same type, e.g., alginate polymers. In an embodiment, the hydrogel capsules are formed by the polymerization of two identical polysaccharides. In an embodiment, the hydrogel capsules are formed by the polymerization of two different polysaccharides. In an embodiment, the hydrogel capsule comprises a plurality of polymers, e.g., a plurality of polysaccharide polymers. In an embodiment, the hydrogel capsule comprises one polysaccharide polymer and a non-polysaccharide polymer.

The hydrogel capsules described herein may be homogenous, i.e., may not comprise a non-polysaccharide polymer. In an embodiment, the hydrogel capsule described herein does not comprise a polymer selected from polyacrylamide, poly(vinyl alcohol), poly(ethylene oxide), polyethylene glycol (PEG), and polyphosphazene. In an embodiment, the hydrogel capsule does not comprise poly(vinyl alcohol) In an embodiment, the hydrogel capsule does not comprise poly(ethylene oxide). In an embodiment, the hydrogel capsule does not comprise polyethylene glycol (PEG). In an embodiment, the hydrogel capsule does not comprise polyphosphazenes.

In an embodiment of the invention, the hydrogel capsules are two-compartment hydrogel capsules. In a preferred embodiment of the invention, the hydrogel capsules consist of an inner compartment and an outer compartment. In an embodiment, the two compartments are formed from the same type of modified polysaccharides. In an embodiment, the two compartments are formed from different types of modified polysaccharides.

In some embodiments, the first and second compartments comprise a blend of polymers (i.e., a mixture of polymers). In some embodiments, the first (inner) compartment comprises a blend of polymers. In some embodiments, the second (outer) compartment comprises a blend of polymers. In some embodiments, the first and second compartments comprise the same blend of polymers. In some embodiments, the first and second compartments comprise different blend of polymers.

In some embodiments, the first compartment comprises a blend of polymers and the second compartment does not comprise a blend of polymers. In some embodiments, the first compartment comprises a blend of polymers and the second compartment comprises a single type of polymer.

In some embodiments, the first compartment does not comprise a blend of polymers and the second compartment comprises a blend of polymers. In some embodiments, the first compartment comprises a single type of polymer and the second compartment comprises a blend of polymers.

In some embodiments, the first and second compartments comprise a blend of alginate polymers. In some embodiments, the first compartment comprises a blend of alginate polymers. In some embodiments, the second compartment comprises a blend of alginate polymers.

In some embodiments of the invention, the first and second compartments comprise a blend of VLVG alginate and SLG100 alginate. In some embodiments of the invention, the first compartment comprises a blend of VLVG alginate and SLG100 alginate. In some embodiments of the invention, the second compartment comprises a blend of VLVG alginate and SLG100 alginate.

In some embodiments, the hydrogel capsule comprises: (i) an inner compartment comprising VLVG alginate, wherein the VLVG alginate comprises a compound of Formula (IV) and the SLG100 alginate comprises a compound of Formula (V); and, (ii) an outer compartment comprising a blend of VLVG and SLG100 alginate, wherein the VLVG alginate comprises a compound of Formula (IV) and the SLG100 alginate comprises a compound of Formula (V).

In some embodiments of the invention, the first and second compartments comprise a blend of VLVG alginate and SLG100 alginate. In some embodiments of the invention, the first compartment comprises a blend of VLVG alginate and SLG100 alginate. In some embodiments of the invention, the second compartment comprises a blend of VLVG alginate and SLG100 alginate.

In some embodiments, the hydrogel capsule comprises: (i) an inner compartment comprising a blend of VLVG and SLG100 alginate, wherein the VLVG alginate comprises a compound of Formula (IV) and the SLG100 alginate comprises a compound of Formula (V); and, (ii) an outer compartment comprising a blend of VLVG and SLG100 alginate, wherein the VLVG alginate comprises a compound of Formula (IV) and the SLG100 alginate comprises a compound of Formula (V).

In some embodiments, the hydrogel capsule comprises: (i) an inner compartment comprising a blend of VLVG and SLG100 alginate, wherein the VLVG alginate comprises a compound of Formula (I) and Formula (IV) and the SLG100 alginate comprises a compound of Formula (V); and, (ii) an outer compartment comprising a blend of VLVG and SLG100 alginate, wherein the VLVG alginate comprises a compound of Formula (I) and Formula (IV) and the SLG100 alginate comprises a compound of Formula (V).

The modified polymer comprising a crosslinking moiety may be able to undergo further polymerization, e.g., may react with compatible functional groups on the same or different polymer. In an embodiment, a polymer modified to include a thiol group and a second polymer modified to include an alkene group such that a crosslinked polymer may be formed by reacting the first and second polymers. In some embodiments, the hydrogel forms by the crosslinking of unsaturated functional groups by a chain-growth polymerization process. In other embodiments the hydrogel forms by crosslinking of unsaturated functional groups by a step-growth polymerization process. The step-growth polymerization process preferably comprises a reaction between one or more unsaturated functional groups (g, alkenyl groups) of one polysaccharide chain and thiolated functional groups of another polymer chain. In some embodiments, the step-growth polymerization a thiol-ene photoclick reaction.

The present disclosure features dual-crosslinked polysaccharide for encapsulating mammalian cells. The dual-crosslinked polysaccharide hydrogels contain least one cell binding substance (CBS) (as defined herein). The cells are capable of expressing a therapeutic agent upon implant of the hydrogel in a subject, e.g., a human or other mammalian subject. In addition, the device comprises at least one means for mitigating the FBR (as defined herein).

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibroic polymer comprises an alginate chemically modified with a Compound of Formula (I). The alginate in the afibrotic polymer may be the same or different than any unmodified alginate that is present in the device. In an embodiment, the density of the Compound of Formula (I) in the afibrotic alginate (e.g, amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

The hydrogel capsules described herein may be porous or non-porous. The pores in a polysaccharide hydrogel (e.g., an alginate hydrogel) function as a selectively permeable membrane to small proteins and molecules while preventing larger, unwanted molecules such as immunoglobins access to encapsulated cells. In a preferred embodiment, the hydrogels and hydrogel capsules described herein are porous. In an embodiment, the hydrogel capsules have an average pore diameter between about 10 nm and about 50 nm. In some embodiments, the average pore diameter is between about 10 nm and 40 nm. In some embodiments, the average pore diameter is between about 10 nm and 30 nm. In some embodiments, the average pore diameter is between 10 and 20 nm.

The physical properties of the hydrogel capsules described herein (e.g., as described in the Examples) control the release of encapsulated molecules (e.g., as determined by a dextran permeability assay). In some embodiments, the average molecular weight permeability is from about 50 kDa to about 400 kDa. In some embodiments, the average molecular weight permeability is from about 100 kDa to about 400 kDa. In some embodiments, the average molecular weight permeability is from about 100 kDa to about 350 kDa. In some embodiments, the average molecular weight permeability is from about 100 kDa to about 300 kDa. In some embodiments, the average molecular weight permeability is from about 100 kDa to about 250 kDa. In some embodiments, the average molecular weight permeability is from about 100 kDa to about 200 kDa. In some embodiments, the average molecular weight permeability is from about 100 kDa to about 150 kDa. In a preferred embodiment, the average molecular weight permeability is from about 125 kDa to about 175 kDa.

The hydrogel capsules described herein may be porous or non-porous. The pores in a polysaccharide hydrogel capsule (e.g., formed from an alginate hydrogel) function as a selectively permeable membrane to small proteins and molecules while preventing larger, unwanted molecules such as immunoglobins access to encapsulated cells. In a preferred embodiment, the hydrogels and hydrogel capsules described herein are porous. In some embodiments, the average pore diameter is about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, or 20 nm. In some embodiments, the average pore diameter is greater than about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, or 20 nm. In some embodiments, the average pore diameter is less than about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, or 20 nm.

In some embodiments, the mean pore size of the first compartment and the second compartment of the particle (e.g., hydrogel capsule) is substantially the same. In some embodiments, the mean pore size of the first compartment and the second compartment of the particle differ by about 1.5%, 2%, 5%7.5% 10%, 15%, 20%, 25%, 30%, 35%, 40%, 45%, 50%, 55%, 60%, 65%, 70%, 75%, or more. In some embodiments, the mean pore size of the particle (e.g., mean pore size of the first compartment and/or mean pore size of the second compartment) is dependent on a number of factors, such as the material(s) within each compartment, the presence and density of the photoactive crosslinker, the presence and density of a compound of Formula (I).

The hydrogel capsules described herein should not have pores of a sufficient diameter to allow for the movement of cells (e.g., immune cells, e.g., dendritic cells) through the hydrogel. In some embodiments, the diameters of the pores are small enough to prevent the movement of antibodies through the hydrogel. In some embodiments, the hydrogel capsules described herein do not have pore sizes greater than 75 μm. In some embodiments, the hydrogel capsules described herein do not have pore sizes greater than 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm or 75 μm.

In some embodiments, the hydrogel capsules described herein may be characterized by their absolute fracture strength (e.g., crush strength) as determined by using a texture analyzer. In some embodiments, the absolute fracture strength is between 50 and 800 g. In some embodiments, the hydrogel capsules described herein have an absolute strength of about 50 g, 60 g, 70 g, 80 g, 90 g, 100 g, 110 g, 120 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, or 800 g. In some embodiments, the hydrogel capsules described herein have an absolute strength of greater than about 50 g, 60 g, 70 g, 80 g, 90 g, 100 g, 110 g, 120 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, or 800 g. In some embodiments, the hydrogel capsules described herein have an absolute strength of less than about 50 g, 60 g, 70 g, 80 g, 90 g, 100 g, 110 g, 120 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, or 800 g.

The present disclosure features particles (e.g., hydrogel capsules) comprising a first compartment, a second compartment, a crosslinking moiety described herein (e.g., a compound of Formula (IV) or (V)), and optionally a compound of Formula (I).

The photoactive crosslinking moiety is covalently bound to a polysaccharide polymer present in the first and/or second compartments. The particle (e.g., hydrogel capsule) may be spherical or have any other shape. The particle (e.g., hydrogel capsule) may comprise materials such as metals, metallic alloys, ceramics, polymers, fibers, inert materials, and combinations thereof. A particle (e.g., hydrogel capsule) may be completely made up of one type of material, or may comprise numerous other materials within the second (outer) compartment and first (inner) compartment.

In some embodiments, the first compartment is modified with a compound of Formula (I). In some embodiments, the second compartment is modified with a compound of Formula (I). In some embodiments, both the first compartment and the second compartment are independently modified with a compound of Formula (I).

In some embodiments, a particle, (e.g., a hydrogel capsule) has a largest linear dimension (LLD), e.g., mean diameter, or size that is greater than 1 millimeter (mm), preferably 1.5 mm or greater. In some embodiments, a particle (e.g., a hydrogel capsule) can be as large as 10 mm in diameter or size. For example, a particle (e.g., a hydrogel capsule) described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm. In some embodiments, the particle (e.g., hydrogel capsule) has a mean diameter or size between 1 mm to 8 mm. In some embodiments, the particle (e.g., hydrogel capsule) has a mean diameter or size between 1 mm to 4 mm. In some embodiments, the particle (e.g., hydrogel capsule) has a mean diameter or size between 1 mm to 2 mm. In some embodiments, the particle (e.g., hydrogel capsule) has a mean diameter or size between 1.5 mm to 2 mm.

In some embodiments, a particle (e.g., hydrogel capsule) has a largest linear dimension (LLD), e.g., mean diameter, or size that is 1 millimeter (mm) or smaller. In some embodiments, the particle (e.g., hydrogel capsule) is in a size range of 0.3 mm to 1 mm, 0.4 mm to 1 mm, 0.5 mm to 1 mm, 0.6 mm to 1 mm, 0.7 mm to 1 mm, 0.8 mm to 1 mm or 0.9 mm to 1 mm.

In some embodiments, the second (outer) compartment completely surrounds the first (inner) compartment, and the inner boundary of the second compartment forms an interface with the outer boundary of the first compartment. In such embodiments, the thickness of the second (outer) compartment means the average distance between the outer boundary of the second compartment and the interface between the two compartments. In some embodiments, the thickness of the outer compartment is greater than about 10 nanometers (nm), preferably 100 nm or greater and can be as large as 1 mm. For example, the thickness of the outer compartment in a particle described herein may be 10 nanometers to 1 millimeter, 100 nanometers to 1 millimeter, 500 nanometers to 1 millimeter, 1 micrometer (μm) to 1 millimeter, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness of the outer compartment is 100 nanometers to 1 millimeters, between 1 μm and 1 mm, between 1 μm and 500 μm or between 5 μm and 1 mm.

In some embodiments, both the first compartment and the second compartment comprise the same polymer. In some embodiments, the first compartment and the second compartment comprise different polymers. In some embodiments, the first compartment comprises an alginate. In some embodiments, the second compartment comprises an alginate. In some embodiments, both the first compartment and the second compartment comprise an alginate. In some embodiments, the alginate in the first compartment is different than the alginate in the second compartment. In some embodiments, the first compartment comprises an alginate and the second compartment comprises a different polymer (e.g., a polysaccharide, e.g., hyaluronate or chitosan). In some embodiments, the second compartment comprises an alginate and the first compartment comprises a different polymer (e.g., a polysaccharide, e.g., hyaluronate or chitosan).

Both the first compartment and the second compartment may include a single component (e.g., one polymer) or more than one component (e.g., a blend of polymers). In some embodiments, the first compartment comprises only alginate (e.g., chemically modified alginate, or a blend of an unmodified alginate and a chemically modified alginate). In some embodiments, the second compartment comprises only alginate (e.g., chemically modified alginate or a blend of an unmodified alginate and a chemically modified alginate). In some embodiments, both the first and the second compartment independently comprise only alginate (e.g., chemically modified alginate or blend of an unmodified alginate and a chemically modified alginate).

In some embodiments, the first and second compartments comprise a blend of polymers (i.e., a mixture of polymers). In some embodiments, the first (inner) compartment comprises a blend of polymers. In some embodiments, the second (outer) compartment comprises a blend of polymers. In some embodiments, the first and second compartments comprise the same blend of polymers. In some embodiments, the first and second compartments comprise different blends of polymers. In some embodiments, at least one polymer in the blend comprising the outer compartment is covalently modified with a photoactive crosslinker described herein (e.g., a compound of Formula (IV), (V) or (VI). In some embodiments, at least one polymer in the blend comprising the second (outer) compartment is covalently modified with an afibrotic compound described herein, e.g., a compound of Formula (I). In some embodiments, at least one polymer in the blend comprising the second (outer) compartment is covalently modified with both a photoactive crosslinker and an afibrotic compound.

In some embodiments, the first compartment comprises a blend of 2, 3, 4, 5, 6, 7, 8, 9, 10 or more polymers. In some embodiments, the first compartment comprises a blend of 2 polymers. In some embodiments, the first compartment comprises a blend of 3 polymers. In some embodiments, the first compartment comprises a blend of 4 polymers. In some embodiments, the first compartment comprises a blend of 5 polymers. In some embodiments, the first compartment comprises a blend of 6 polymers. In some embodiments, the first (inner) compartment comprises a blend of 7 polymers. In some embodiments, the first (inner) compartment comprises a blend of 8 polymers. In some embodiments, the first (inner) compartment comprises a blend of 9 polymers. In some embodiments, the first (inner) compartment comprises a blend of 10 polymers.

In some embodiments, the second (outer) compartment comprises a blend of 2, 3, 4, 5, 6, 7, 8, 9, 10 or more polymers. In some embodiments, the second (outer) compartment comprises a blend of 2 polymers. In some embodiments, the second (outer) compartment comprises a blend of 3 polymers. In some embodiments, the second (outer) compartment comprises a blend of 4 polymers. In some embodiments, the second (outer) compartment comprises a blend of 5 polymers. In some embodiments, the second (outer) compartment comprises a blend of 6 polymers. In some embodiments, the second (outer) compartment comprises a blend of 7 polymers. In some embodiments, the second (outer) compartment comprises a blend of 8 polymers. In some embodiments, the second (outer) compartment comprises a blend of 9 polymers. In some embodiments, the second (outer) compartment comprises a blend of 10 polymers.

In some embodiments, the first compartment comprises a blend of polymers and the second compartment does not comprise a blend of polymers. In some embodiments, the first compartment comprises a blend of polymers and the second compartment comprises a single type of polymer.

In some embodiments, the first compartment does not comprise a blend of polymers and the second compartment comprises a blend of polymers. In some embodiments, the first compartment comprises a single type of polymer and the second compartment comprises a blend of polymers.

In some embodiments, the first and second compartments comprise a blend of polymers and the polymers of the blend are any two miscible polymers.

In some embodiments, the first and second compartments comprise a blend of polymers and the polymers are selected from the group consisting of: alginate, hyaluronate, and chitosan.

In some embodiments, the first and second compartments comprise a blend of polymers and the polymers are selected from the group consisting of: alginate, hyaluronate, and chitosan. In some embodiments, the first compartment comprises a blend of polymers and the polymers are selected from the group consisting of: alginate, hyaluronate, and chitosan. In some embodiments, the second compartment comprises a blend of polymers and the polymers are selected from the group consisting of: alginate, hyaluronate, and chitosan.

In some embodiments, the first and second compartments comprise a blend of alginate polymers. In some embodiments, the first compartment comprises a blend of alginate polymers. In some embodiments, the second compartment comprises a blend of alginate polymers.

In some embodiments, the first and second compartments comprise a blend of alginate polymers and the alginate polymers are selected from high-guluronic acid alginate and high-mannuronic acid alginate. In some embodiments, the first compartment comprises a blend of alginate polymers and the alginate polymers are selected from high-guluronic acid alginate and high-mannuronic acid alginate. In some embodiments, the second compartment comprises a blend of alginate polymers and the alginate polymers are selected from high-guluronic acid alginate and high-mannuronic acid alginate.

In some embodiments, the first and second compartments comprise a blend of alginate polymers and the alginate polymers are selected from low molecular weight alginate, medium molecular weight alginate, high molecular weight alginate, and ultra-high molecular weight alginate. In some embodiments, the first compartment comprises a blend of alginate polymers and the alginate polymers are selected from low molecular weight alginate, medium molecular weight alginate, high molecular weight alginate, and ultra-high molecular weight alginate. In some embodiments, the second compartment comprises a blend of alginate polymers and the alginate polymers are selected from low molecular weight alginate, medium molecular weight alginate, high molecular weight alginate, and ultra-high molecular weight alginate.

In some embodiments, the first and second compartments comprise a blend of alginate polymers and the alginate polymers are selected from Kimica Algin IL-2, Kimica Algin IL-6, Kimica Algin I-1, Kimica Algin I-3, Kimica Algin I-5, Kimica Algin I-8, Kimica Algin LZ-2, Kimica Algin ULV-L3, Kimica Algin ULV-L5, Kimica Algin ULV-1G, Kimica Algin ULV-5G, Kimica Algin ULV IL-6G, Pronova UP VLVM, Pronova UP LVM, Pronova UP MVM, Pronova UP VLVG, Pronova UP MVG, Pronova UP LVG, Pronova SLM20, Pronova SLM100, Pronova SLG20, and Pronova SLG100. In some embodiments, the first compartment comprises a blend of alginate polymers and the alginate polymers are selected from Kimica Algin IL-2, Kimica Algin IL-6, Kimica Algin I-1, Kimica Algin I-3, Kimica Algin I-5, Kimica Algin I-8, Kimica Algin LZ-2, Kimica Algin ULV-L3, Kimica Algin ULV-L5, Kimica Algin ULV-1G, Kimica Algin ULV-5G, Kimica Algin ULV IL-6G, Pronova UP VLVM, Pronova UP LVM, Pronova UP MVM, Pronova UP VLVG, Pronova UP MVG, Pronova UP LVG, Pronova SLM20, Pronova SLM100, Pronova SLG20, and Pronova SLG100. In some embodiments, the second compartment comprises a blend of alginate polymers and the alginate polymers are selected from Kimica Algin IL-2, Kimica Algin IL-6, Kimica Algin I-1, Kimica Algin I-3, Kimica Algin I-5, Kimica Algin I-8, Kimica Algin LZ-2, Kimica Algin ULV-L3, Kimica Algin ULV-L5, Kimica Algin ULV-JG, Kimica Algin ULV-5G, Kimica Algin ULV IL-6G, Pronova UP VLVM, Pronova UP LVM, Pronova UP MVM, Pronova UP VLVG, Pronova UP MVG, Pronova UP LVG, Pronova SLM20, Pronova SLM100, Pronova SLG20, and Pronova SLG100.

In some embodiments, the first and second compartments comprise a blend of two alginate polymers at any ratio. In some embodiments, the ratio of the two alginate polymers in the blend is about 99:1, 95:5, 90:10, 85:15, 80:20, 75:25, 70:30, 65:35, 60:40, 55:45, or 50:50. In some embodiments, the ratio of the two alginate polymers in the blend is about 99:1. In some embodiments, the ratio of the two alginate polymers in the blend is about 95:5. In some embodiments, the ratio of the two alginate polymers in the blend is about 90:10. In some embodiments, the ratio of the two alginate polymers in the blend is about 85:15. In some embodiments, the ratio of the two alginate polymers in the blend is about 80:20. In some embodiments, the ratio of the two alginate polymers in the blend is about 75:25. In some embodiments, the ratio of the two alginate polymers in the blend is about 70:30. In some embodiments, the ratio of the two alginate polymers in the blend is about 65:35. In some embodiments, the ratio of the two alginate polymers in the blend is about 60:40. In some embodiments, the ratio of the two alginate polymers in the blend is about 55:45. In some embodiments, the ratio of the two alginate polymers in the blend is about 50:50.

In some embodiments, the first and second compartments comprise a blend of two alginate polymers at any ratio. In some embodiments, the ratio of the two alginate polymers in the blend is greater than about 99:1, 95:5, 90:10, 85:15, 80:20, 75:25, 70:30, 65:35, 60:40, 55:45, or 50:50. In some embodiments, the ratio of the two alginate polymers in the blend is greater than about 99:1. In some embodiments, the ratio of the two alginate polymers in the blend is greater than about 95:5. In some embodiments, the ratio of the two alginate polymers in the blend is greater than about 90:10. In some embodiments, the ratio of the two alginate polymers in the blend is greater than about 85:15. In some embodiments, the ratio of the two alginate polymers in the blend is greater than about 80:20. In some embodiments, the ratio of the two alginate polymers in the blend is greater than about 75:25. In some embodiments, the ratio of the two alginate polymers in the blend is greater than about 70:30. In some embodiments, the ratio of the two alginate polymers in the blend is greater than about 65:35. In some embodiments, the ratio of the two alginate polymers in the blend is greater than about 60:40. In some embodiments, the ratio of the two alginate polymers in the blend is greater than about 55:45. In some embodiments, the ratio of the two alginate polymers in the blend is greater than about 50:50.

In some embodiments, a polymer of the first compartment of the particle (e.g., hydrogel capsule) is modified with one compound of Formula (I), and a polymer of the second compartment of the particle (e.g., hydrogel capsule) is modified with a different compound of Formula (I). In some embodiments, the particle (e.g., hydrogel capsule) comprises a mixture of polymers modified with a compound of Formula (I) and unmodified polymers (e.g., polymers not modified with a compound of Formula (I)). In some embodiments, the first compartment comprises a mixture (i.e., a blend) of polymers modified with a compound of Formula (I) and unmodified polymers (e.g., polymers not modified with a compound of Formula (I)). In some embodiments, the second compartment comprises a mixture of polymers modified with a compound of Formula (I) and unmodified polymers (e.g., polymers not modified with a compound of Formula (I)).

A polymer of a particle (e.g., hydrogel capsule) described herein may be modified with a compound of Formula (I) or a pharmaceutically acceptable salt thereof on one or more monomers of the polymer. The modified polymer of the particle (e.g., hydrogel capsule) may be present in the first (inner) compartment of the particle, the second (outer) compartment of the particle, or both the first (inner) and second (outer) compartments of the particle. In some embodiments, the modified polymer is present only in the second compartment (which includes the exterior particle surface). In some embodiments, at least 0.5% of the monomers of a polymer are modified with a compound of Formula (I) (e.g., at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of the monomers of a polymer are modified with a compound of Formula (I)). In some embodiments, 0.5% to 50%, 10% to 90%, 10% to 50%, or 25-75%, of the monomers of a polymer are modified with a compound of Formula (I). In some embodiments, 1% to 20% of the monomers of a polymer are modified with a compound of Formula (I). In some embodiments, 1% to 10% of the monomers of a polymer are modified with a compound of Formula (I).

In some embodiments, the polymer (e.g., alginate) (when modified with a compound of Formula (I), e.g., Compound 101 of Table 3) comprises an increase in % N (as compared with unmodified polymer, e.g., alginate) of any of the following values: (i) at least 0.1%, 0.2%, 0.5%, 1.0%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% N by weight; (ii) 0.1% to 10% by weight, (iii) 0.1% to 2% N by weight; (iv) 2% to 4% N by weight; (v) 4% to 8% N by weight; (vi) 5% to 9% N by weight; (vii) 6% to 9% N by weight, (viii) 6% to 8% N by weight; (ix) 7% to 9% N by weight; and (x) 8% to 9% N by weight where, in each case, % N is determined by combustion analysis (e.g., as described in Example 2 herein) and corresponds to the amount of compound of Formula (I) in the modified polymer.

A particle (e.g., hydrogel capsule) (e.g., a first compartment or second compartment therein) may comprise a compound of Formula (I) in an amount that confers a specific feature to the particle. For example, the particle surface (e.g., the exterior of the outer compartment) may comprise a concentration or density of a compound of Formula (I) such that the particle is afibrotic (i.e., mitigates the foreign body response) in a subject. In an embodiment, the particle surface comprises an alginate chemically modified with an afibrotic-effective amount of Compound 101. In an embodiment, the afibrotic-effective amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis (e.g., as described in Example 2 herein) and corresponds to the amount of Compound 101 in the modified alginate.

In an embodiment, mechanical testing of hydrogel capsules is performed on a TA.XT plus Texture Analyzer (Stable Micro Systems, Surrey, United Kingdom) using a 5 mm probe attached to a 5 kg load cell. Individual capsules are placed on a platform and are compressed from above by the probe at a fixed rate of 0.5 mm/sec. Contact between the probe and capsule is detected when a repulsive force of 1 g is measured. The probe continues to travel 90% of the distance between the contact height of the probe and the platform, compressing the capsule to the point of bursting. The resistance to the compressive force of the probe is measured and can be plotted as a function of probe travel (force v. displacement curve). Typically, before a capsule bursts completely it will fracture slightly and the force exerted against the probe will decrease a small amount. An analysis macro can be programmed to detect the first time a decrease of 0.25-0.5 g occurs in the force v. displacement curve. The force applied by the probe when this occurs is termed the initial fracture force. In an embodiment, the desired mechanical strength of a particle described herein (e.g., a two-compartment hydrogel capsule) has an initial fracture force of greater than 1, 1.5, 2, 2.5 or 3 grams or at least 2 grams.

In an embodiment, the desired mechanical strength of a particle (e.g., hydrogel capsule) is the ability to remain intact at a desired timepoint after implantation in a subject, e.g., both the outer and inner compartments of a hydrogel capsule removed from a subject are visibly intact after retrieval from an immune competent mouse when observed by optical microscopy, e.g., by brightfield imaging as described in the Examples herein.

In an embodiment, the particle surface comprises an alginate chemically modified with Compound 101 in an amount that provides the particle with both an afibrotic property and a desired mechanical strength, e.g., a concentration or density of Compound 101 in the modified alginate that produces an increase in % N (as compared with the unmodified alginate) of any of the following values: (i) 1% to 3% by weight, (ii) 2% to 4% N by weight; (iii) 4% to 8% N by weight; (iv) 5% to 9% N by weight; (v) 6% to 9% N by weight, (vi) 6% to 8% N by weight; (vii) 7% to 9% N by weight; and (ix) 8% to 9% N by weight; where, in each case, % N is determined by combustion analysis (e.g., as described in Example 2 herein) and corresponds to the amount of compound of Formula (I) in the modified alginate.

When a particle (e.g., a first compartment or second compartment therein) comprises alginate, the alginate can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) dissolved in a buffer or solvent, such as acetonitrile (0.3 M). The reaction may be warmed, e.g., to 55° C. for 16 h, then cooled to room temperature and concentrated via rotary evaporation. The residue may then be dissolved in a buffer or solvent, e.g., water. The mixture may then be filtered, e.g., through a bed of cyano-modified silica gel (Silicycle) and the filter cake washed with water. The resulting solution may then be dialyzed (10,000 MWCO membrane) against a buffer or water for 24 hours, e.g., replacing the buffer or water at least one time, at least two times, at least three times, or more. The resulting solution can be concentrated, e.g., via lyophilization, to afford the desired chemically modified alginate.

In some embodiments, a particle described herein comprises a cell. In some embodiments, the cell is engineered to produce a therapeutic agent (e.g., a protein or polypeptide, e.g., an antibody, protein, enzyme, or growth factor). In some embodiments, the cell is disposed with the first compartment. In some embodiments, the cell is disposed within the second compartment. In some embodiments, the cell is disposed in the first compartment and the second compartment does not comprise a cell. A particle (e.g., hydrogel capsule) may comprise an active or inactive fragment of a protein or polypeptide, such as glucose oxidase (e.g., for glucose sensor), kinase, phosphatase, oxygenase, hydrogenase, reductase.

A particle (e.g., hydrogel capsule) described herein may be configured to release a therapeutic agent, e.g., an exogenous substance, e.g., a therapeutic agent described herein. In some embodiments, the therapeutic agent is a compound of Formula (I) or a pharmaceutically acceptable salt thereof. In some embodiments, the therapeutic agent is a biological material. In some embodiments, the therapeutic agent is a nucleic acid (e.g., an RNA or DNA), protein (e.g., a hormone, enzyme, antibody, antibody fragment, antigen, or epitope), small molecule, lipid, drug, vaccine, or any derivative thereof.

A particle (e.g., hydrogel capsule) (e.g., as described herein) may be provided as a preparation or composition for implantation or administration to a subject. In some embodiments, at least 20%, 30%, 35%, 40%, 45%, 50%, 55%, 60%, 65%, 70%, 75%, 80%, 85%, 90%, 95% or 100% of the particles (e.g., hydrogel capsules) in a preparation or composition have a characteristic as described herein, e.g., mean diameter or mean pore size.

### Cells and Therapeutic Agents

The hydrogel capsules of the present disclosure may comprise a wide variety of different cell types (e.g., human cells), including but not limited to: adipose cells, epidermal cells, epithelial cells, endothelial cells, fibroblast cells, islet cells, mesenchymal stem cells, pericytes, subtypes of any of the foregoing, cells derived from any of the foregoing, cells derived from induced pluripotent stem cells and mixtures of one or more of any of the foregoing. Exemplary cell types include the cell types recited in WO 2017/075631 and WO 2019/195055. In an embodiment, the hydrogel capsules described herein comprise a plurality of cells. In an embodiment, the plurality of cells is in the form of a cell suspension prior to being encapsulated within a hydrogel capsule described herein. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers. In some embodiments, the hydrogel capsule does not comprise any islet cells and does not comprise any cells that are capable of producing insulin in a glucose-responsive manner.

The hydrogel capsules of the present disclosure decrease immune cell adhesion compared to an untreated control. In an embodiment, the hydrogel capsules decrease macrophage adhesion compared to an untreated control. In an embodiment, the decrease in macrophage adhesion is between about 1 fold and 10 fold less than an untreated control. In an embodiment, the decrease in macrophage adhesion is between about 1-fold and 8 fold less than an untreated control. In an embodiment, the decrease in macrophage adhesion is between about 1-fold and 7 fold less than an untreated control. In an embodiment, the decrease in macrophage adhesion is between about 1-fold and 6 fold less than an untreated control. In an embodiment, the decrease in macrophage adhesion is between about 1-fold and 5 fold less than an untreated control.

The hydrogels or hydrogel capsules of the present disclosure allow encapsulated cells (e.g., engineered cells) to retain viability (e.g., as determined by a cell viability assay). In some embodiments, the hydrogel or hydrogel capsule allows encapsulated cells to retain viability for at least seven days, at least one month, or at least one year.

The present disclosure features a cell that produces or is capable of producing a therapeutic agent for the prevention or treatment of a disease, disorder, or condition described herein. In an embodiment, the cell is an engineered cell. In an embodiment, the cell is engineered to sense a stimulus, e.g., a chemical signal, and express the therapeutic agent in response to the stimulus. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule, each of which are further elaborated below. Exemplary therapeutic agents include the agents listed in WO 2017/075631 and WO 2019/195055.

In some embodiments, the cells (e.g., engineered cells) produce a nucleic acid. A nucleic acid produced by a cell described herein may vary in size and contain one or more nucleosides or nucleotides, e.g., greater than 2, 3, 4, 5, 10, 25, 50, or more nucleosides or nucleotides. In some embodiments, the nucleic acid is a short fragment of RNA or DNA, e.g., and may be used as a reporter or for diagnostic purposes. Exemplary nucleic acids include a single nucleoside or nucleotide (e.g., adenosine, thymidine, cytidine, guanosine, uridine monophosphate, inosine monophosphate), RNA (e.g., mRNA, siRNA, miRNA, RNAi), and DNA (e.g., a vector, chromosomal DNA). In some embodiments, the nucleic acid has an average molecular weight (in kD) of about 0.25, 0.5, 1, 1.5, 2, 2.5, 5, 10, 25, 50, 100, 150, 200 or more.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by a cell in an implantable element can have a naturally occurring amino acid sequence, or may contain a variant of the naturally occurring sequence. The variant can be a naturally occurring or non-naturally occurring amino acid substitution, mutation, deletion or addition relative to the reference naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a peptide or polypeptide (e.g., a protein) for use with the present invention may be modified in some way, e.g., via chemical or enzymatic modification (e.g., glycosylation, phosphorylation). In some embodiments, the peptide has about 2, 3, 4, 5, 6, 7, 8, 9, 10, 12, 14, 16, 18, 20, 25, 30, 35, 40, 45, or 50 amino acids. In some embodiments, the protein has an average molecular weight (in kD) of 5, 10, 25, 50, 100, 150, 200, 250, 500 or more.

In some embodiments, the protein is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone. In some embodiments, the protein is insulin (e.g., insulin A-chain, insulin B-chain, or proinsulin). In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone. In some embodiments, the protein is not insulin (e.g., insulin A-chain, insulin B-chain, or proinsulin).

In some embodiments, the protein is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor, prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is an antibody molecule. As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a monospecific antibody molecule and binds a single epitope, e.g., a monospecific antibody molecule having a plurality of immunoglobulin variable domain sequences, each of which binds the same epitope. In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

Various types of antibody molecules may be produced by a cell in an implantable element described herein, including whole immunoglobulins of any class, fragments thereof, and synthetic proteins containing at least the antigen binding variable domain of an antibody. The antibody molecule can be an antibody, e.g., an IgG antibody, such as IgG₁, IgG₂, IgG₃, or IgG₄. An antibody molecule can be in the form of an antigen binding fragment including a Fab fragment, F(ab′)2 fragment, a single chain variable region, and the like. Antibodies can be polyclonal or monoclonal (mAb). Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. In some embodiments, the antibody molecule is a single-domain antibody (e.g., a nanobody). The described antibodies can also be modified by recombinant means, for example by deletions, additions or substitutions of amino acids, to increase efficacy of the antibody in mediating the desired function. Exemplary antibodies include anti-beta-galactosidase, anti-collagen, anti-CD14, anti-CD20, anti-CD40, anti-HER2, anti-IL-1, anti-IL-4, anti-IL6, anti-IL-13, anti-IL17, anti-IL18, anti-IL-23, anti-IL-28, anti-IL-29, anti-IL-33, anti-EGFR, anti-VEGF, anti-CDF, anti-flagellin, anti-IFN-α, anti-IFN-β, anti-IFN-γ, anti-mannose receptor, anti-VEGF, anti-TLR1, anti-TLR2, anti-TLR3, anti-TLR4, anti-TLR5, anti-TLR6, anti-TLR9, anti-PDF, anti-PD1, anti-PDL-1, or anti-nerve growth factor antibody. In some embodiments, the antibody is an anti-nerve growth factor antibody (e.g., fulranumab, fasinumab, tanezumab).

In some embodiments, the protein is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of a polypeptide (e.g., a protein) produced by a cell in an implantable element described herein also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, TL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fasl, Fgf2, Flt3l, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, mullerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the protein is a replacement therapy or a replacement protein. In some embodiments, the replacement therapy or replacement protein is a clotting factor or a coagulation factor, e.g., Factor VIII (e.g., comprises a naturally occurring human Factor VIII amino acid sequence or a variant thereof) or Factor IX (e.g., comprises a naturally occurring human Factor IX amino acid sequence or a variant thereof).

In some embodiments, the cell is engineered to express a Factor VIII, e.g., a recombinant Factor VIII. In some embodiments, the cell is derived from human tissue and is engineered to express a Factor VIII, e.g., a recombinant Factor VIII. In some embodiments, the recombinant Factor VIII is a B-domain-deleted recombinant Factor VIII (FVIII-BDD).

In some embodiments, the cell is derived from human tissue and is engineered to express a Factor IX, e.g., a recombinant Factor IX. In some embodiments, the cell is engineered to express a Factor IX, e.g., a wild-type human Factor IX (FIX), or a polymorphic variant thereof. In some embodiments, the cell is engineered to express a gain-in-function (GIF) variant of a wild-type FIX protein (FIX-GIF), wherein the GIF variant has higher specific activity than the corresponding wild-type FIX.

In some embodiments, the replacement therapy or replacement protein is an enzyme, e.g., alpha-galactosidase, alpha-L-iduronidase (IDUA), or N-sulfoglucosamine sulfohydrolase (SGSH). In some embodiments, the replacement therapy or replacement protein is an enzyme, e.g., an alpha-galactosidase A (e.g., comprises a naturally-occurring human alpha-galactosidase A amino acid sequence or a variant thereof). In some embodiments, the replacement therapy or replacement protein is a cytokine or an antibody.

In some embodiments, the therapeutic agent is a sugar, e.g., monosaccharide, disaccharide, oligosaccharide, or polysaccharide. In some embodiments, a sugar comprises a triose, tetrose, pentose, hexose, or heptose moiety. In some embodiments, the sugar comprises a a linear monosaccharide or a cyclized monosaccharide. In some embodiments, the sugar comprises a glucose, galactose, fructose, rhamnose, mannose, arabinose, glucosamine, galactosamine, sialic acid, mannosamine, glucuronic acid, galactosuronic acid, mannuronic acid, or guluronic acid moiety. In some embodiments, the sugar is attached to a protein (e.g., an N-linked glycan or an O-linked glycan). Exemplary sugars include glucose, galactose, fructose, mannose, rhamnose, sucrose, ribose, xylose, sialic acid, maltose, amylose, inulin, a fructooligosaccharide, galactooligosaccharide, a mannan, a lectin, a pectin, a starch, cellulose, heparin, hyaluronic acid, chitin, amylopectin, or glycogen. In some embodiments, the therapeutic agent is a sugar alcohol.

In some embodiments, the therapeutic agent is a lipid. A lipid may be hydrophobic or amphiphilic, and may form a tertiary structure such as a liposome, vesicle, or membrane or insert into a liposome, vesicle, or membrane. A lipid may comprise a fatty acid, glycerolipid, glycerophospholipid, sterol lipid, prenol lipid, sphingolipid, saccharolipid, polyketide, or sphingolipid. Examples of lipids produced by a cell described herein include anandamide, docosahexaenoic acid, aprostaglandin, a leukotriene, a thromboxane, an eicosanoid, a triglyceride, a cannabinoid, phosphatidylcholine, phosphatidylethanolamine, a phosphatidylinositol, a phosohatidic acid, a ceramide, a sphingomyelin, a cerebroside, a ganglioside, estrogen, androsterone, testosterone, cholesterol, a carotenoid, a quinone, a hydroquinone, or a ubiquinone.

In some embodiments, the therapeutic agent is a small molecule. A small molecule may include a natural product produced by a cell. In some embodiments, the small molecule has poor availability or does not comply with the Lipinski rule of five (a set of guidelines used to estimate whether a small molecule will likely be an orally active drug in a human; see, e.g., Lipinski, C. A. et al (2001) Adv Drug Deliv 46:2-36). Exemplary small molecule natural products include an anti-bacterial drug (e.g., carumonam, daptomycin, fidaxomicin, fosfomycin, ispamicin, micronomicin sulfate, miocamycin, mupiocin, netilmicin sulfate, teicoplanin, thienamycin, rifamycin, erythromycin, vancomycin), an anti-parasitic drug (e.g., artemisinin, ivermectin), an anticancer drug (e.g., doxorubicin, aclarubicin, aminolaevulinic acid, arglabin, omacetaxine mepesuccinate, paclitaxel, pentostatin, peplomycin, romidepsin, trabectdin, actinomycin D, bleomycin, chromomycin A, daunorubicin, leucovorin, neocarzinostatin, streptozocin, trabectedin, vinblastine, vincristine), anti-diabetic drug (e.g., voglibose), a central nervous system drug (e.g., L-dopa, galantamine, zicontide), a statin (e.g., mevastatin), an anti-fungal drug (e.g., fumagillin, cyclosporin), 1-deoxynojirimycin, and theophylline, sterols (cholesterol, estrogen, testosterone). Additional small molecule natural products are described in Newman, D. J. and Cragg, M. (2016) J Nat Prod 79:629-661 and Butler, M. S. et al (2014) Nat Prod Rep 31:1612-1661.

In some embodiments, the cell is engineered to synthesize a non-protein or non-peptide small molecule. For example, in an embodiment a cell can produce a statin (e.g., taurostatin, pravastatin, fluvastatin, or atorvastatin).

In some embodiments, the therapeutic agent is an antigen (e.g., a viral antigen, a bacterial antigen, a fungal antigen, a plant antigen, an environmental antigen, or a tumor antigen). An antigen is recognized by those skilled in the art as being immunostimulatory, i.e., capable of stimulating an immune response or providing effective immunity to the organism or molecule from which it derives. An antigen may be a nucleic acid, peptide, protein, sugar, lipid, or a combination thereof.

The cells, e.g., engineered cells, e.g., engineered cells described herein, may produce a single therapeutic agent or a plurality of therapeutic agents. In some embodiments, the cells produce a single therapeutic agent. In some embodiments, a cluster of cells comprises cells that produce a single therapeutic agent. In some embodiments, at least about 1 percent, or about 5, 10, 20, 25, 30, 40, 50, 60, 70, 80, 90, 95, or 99 percent of the cells in a cluster produce a single therapeutic agent (e.g., a therapeutic agent described herein). In some embodiments, the cells produce a plurality of therapeutic agents, e.g., at least 2, 3, 4, 5, 6, 7, 8, 9, or 10 therapeutic agents. In some embodiments, a cluster of cells comprises cells that produce a plurality of therapeutic agents. In some embodiments, at least about 1 percent, or about 5, 10, 20, 25, 30, 40, 50, 60, 70, 80, 90, 95, or 99 percent of the cells in a cluster produce a plurality of therapeutic agents (e.g., a therapeutic agent described herein).

The therapeutic agents may be related or may form a complex. In some embodiments, the therapeutic agent secreted or released from a cell in an active form. In some embodiments, the therapeutic agent is secreted or released from a cell in an inactive form, e.g., as a prodrug. In the latter instance, the therapeutic agent may be activated by a downstream agent, such as an enzyme. In some embodiments, the therapeutic agent is not secreted or released from a cell, but is maintained intracellularly. For example, the therapeutic agent may be an enzyme involved in detoxification or metabolism of an unwanted substance, and the detoxification or metabolism of the unwanted substance occurs intracellularly.

In some embodiments, the hydrogel capsules described herein comprise mammalian cells at a concentration of about 1, 2, 3, 4, 5, 6, 7, 8, 9, 10, 15, 20, 25, 30, 35, 40, 45, or 50 M mL⁻¹. In some embodiments, the hydrogel capsules described herein comprise mammalian cells at a concentration of about 1 M mL⁻¹. In some embodiments, the hydrogel capsules described herein comprise mammalian cells at a concentration of about 2 M mL⁻¹. In some embodiments, the hydrogel capsules described herein comprise mammalian cells at a concentration of about 3 M mL⁻¹. In some embodiments, the hydrogel capsules described herein comprise mammalian cells at a concentration of about 4 M mL⁻¹. In some embodiments, the hydrogel capsules described herein comprise mammalian cells at a concentration of about 5 M mL⁻¹. In some embodiments, the hydrogel capsules described herein comprise mammalian cells at a concentration of about 6 M mL⁻¹. In some embodiments, the hydrogel capsules described herein comprise mammalian cells at a concentration of about 7 M mL⁻¹. In some embodiments, the hydrogel capsules described herein comprise mammalian cells at a concentration of about 8 M mL⁻¹. In some embodiments, the hydrogel capsules described herein comprise mammalian cells at a concentration of about 9 M mL¹. In some embodiments, the hydrogel capsules described herein comprise mammalian cells at a concentration of about 10 M mL⁻¹. In some embodiments, the hydrogel capsules described herein comprise mammalian cells at a concentration of about 15 M mL⁻¹. In some embodiments, the hydrogel capsules described herein comprise mammalian cells at a concentration of about 20 M mL⁻¹. In some embodiments, the hydrogel capsules described herein comprise mammalian cells at a concentration of about 25 M mL⁻¹. In some embodiments, the hydrogel capsules described herein comprise mammalian cells at a concentration of about 30 M mL⁻¹. In some embodiments, the hydrogel capsules described herein comprise mammalian cells at a concentration of about 35 M mL⁻¹. In some embodiments, the hydrogel capsules described herein comprise mammalian cells at a concentration of about 40 M mL⁻¹. In some embodiments, the hydrogel capsules described herein comprise mammalian cells at a concentration of about 45 M mL⁻¹. In some embodiments, the hydrogel capsules described herein comprise mammalian cells at a concentration of about 50 M mL⁻¹.

In some embodiments, the hydrogel capsules described herein comprise mammalian cells at a concentration between about 1-50 M mL⁻¹, 1-45 M mL⁻¹, 1-40 M mL⁻¹, 1-35 M mL⁻¹, 1-30 M mL⁻¹, 1-25 M mL⁻¹, 1-20 M mL⁻¹, 1-15 M mL⁻¹, 1-10 M mL⁻¹, 1-5 M mL⁻¹, 5-50 M mL⁻¹, 5-45 M mL⁻¹, 5-40 M mL⁻¹, 5-35 M mL⁻¹, 5-30 M mL⁻¹, 5-25 M mL⁻¹, 5-20 M mL⁻¹, 5-15 M mL⁻¹, 5-10 M mL⁻¹, 10-50 M mL⁻¹, 10-45 M mL⁻¹, 10-40 M mL⁻¹, 10-35 M mL⁻¹, 10-30 M mL⁻¹, 10-25 M mL⁻¹, 10-20 M mL⁻¹, 10-15 M mL⁻¹, 15-50 M mL⁻¹, 15-45 M mL⁻¹, 15-40 M mL⁻¹, 15-35 M mL⁻¹, 15-30 M mL⁻¹, 15-25 M mL⁻¹, 15-20 M mL⁻¹, 20-50 M mL⁻¹, 20-45 M mL⁻¹, 20-40 M mL⁻¹, 20-35 M mL⁻¹, 20-30 M mL⁻¹, or 20-25 M mL⁻¹.

### Methods of Treatment

Described herein are methods for preventing or treating a disease, disorder, or condition in a subject through administration or implantation of a hydrogel capsule comprising (i) a polysaccharide polymer described herein and (ii) an islet cell. In some embodiments, the methods described herein directly or indirectly reduce or alleviate at least one symptom of a disease, disorder, or condition (e.g., Type 1 diabetes). In some embodiments, the methods described herein prevent or slow the onset of a disease, disorder, or condition (e.g., Type 1 diabetes). In some embodiments, the subject is a human.

In some embodiments, the disease, disorder, or condition affects a system of the body, e.g. the nervous system (e.g., peripheral or central nervous system), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is an autoimmune disease. In some embodiments, the disease, disorder, or condition is diabetes (Type 1 or Type 2).

The present disclosure further comprises methods for identifying a subject having or suspected of having a disease, disorder, or condition described herein (e.g., Type 1 diabetes), and upon such identification, administering to the subject an a hydrogel capsule comprising (i) a polysaccharide polymer described herein and (ii) an islet cell, e.g., wherein the hydrogel capsule is optionally modified with a compound of Formula (I), or a composition thereof. In an embodiment, the subject has or is diagnosed with having diabetes (e.g., Type 1 diabetes). The subject may have any biomarker or other diagnostic criteria associated with diabetes, such as a high blood glucose level (e.g., greater than 300 mg/dL, greater than 400 mg/dL) or a high hemoglobin A1C level (e.g., a hemoglobin A1C level greater than 5.9%, a hemoglobin A1C level greater than 6.5%, a hemoglobin A1C level greater than 7%). In an embodiment, the subject is a human. In an embodiment, the subject is an adult. In an embodiment, the subject is a child (e.g., a subject less than 21 years of age, less than 18 years of age, less than 15 years of age, less than 12 years of age, less than 10 years of age, or less than 6 years of age).

### Methods of Making Particles

The present disclosure further comprises methods for making a particle described herein, e.g., a particle comprising a first compartment, a second compartment, and a compound of Formula (I). In some embodiments where the particle is a hydrogel capsule, the method of making the particle comprises contacting a plurality of droplets comprising first and second polymer solutions (e.g., each comprising a hydrogel-forming polymer) with an aqueous cross-linking solution. The droplets can be formed using any technique known in the art.

Each compartment of a particle described herein may comprise an unmodified polymer, a polymer modified with a compound of Formula (I), a polymer modified with a crosslinker, or a blend thereof. Briefly, in performing a method of preparing a particle configured as a two-compartment hydrogel capsule, a volume of a first polymer solution (e.g., comprising an unmodified polymer, a polymer modified with a compound of Formula (I), a polymer modified with a crosslinker, or a blend thereof, and optionally containing cells,) is loaded into a first syringe connected to the inner lumen of a coaxial needle. The first syringe may then be connected to a syringe pump oriented vertically above a vessel containing an aqueous cross-linking solution which comprises a cross-linking agent, a buffer, and an osmolarity-adjusting agent. A volume of the second polymer solution (e.g., comprising an unmodified polymer, a polymer modified with a compound of Formula (I), a polymer modified with a crosslinker, or a blend thereof, and optionally containing cells) is loaded into a second syringe connected to the outer lumen of the coaxial needle. The second syringe may then be connected to a syringe pump oriented horizontally with respect to the vessel containing the cross-linking solution. A high voltage power generator may then be connected to the top and bottom of the needle. The syringe pumps and power generator can then be used to extrude the first and second polymer solutions through the syringes with settings determined to achieve a desired droplet rate of polymer solution into the cross-linking solution. The skilled artisan may readily determine various combinations of needle lumen sizes, voltage range, flow rates, droplet rate and drop distance to create 2-compartment hydrogel capsule compositions in which the majority (e.g., at least 80%, 85%, 90% or more) of the capsules are within 10% of the target size and have a sphere-like in shape. After exhausting the first and second volumes of polymer solution, the droplets may be allowed to cross-link in the cross-linking solution for certain amount of time, e.g., about five minutes.

Exemplary process parameters for preparing a composition of millicapsules (e.g., 1.5 mm diameter millicapsules) include the following. A coaxial needle is disposed above the surface of the cross-linking solution at a distance sufficient to provide a drop distance from the needle tip to the solution surface. In an embodiment, the distance between the needle tip and the solution surface is between 1 to 5 cm. In an embodiment, the first and second polymer solutions are extruded through the needle with a total flow rate of between 0.05 mL/min to 5 mL/min, or 0.05 mL/min to 2.5 mL/min, or 0.05 mL/min to about 1 mL/min, or 0.05 mL/min to 0.5 mL/min, or 0.1 mL/min to 0.5 mL/min. In an embodiment, the first and second polymer solutions are extruded through the needle with a total flow rate of about 0.05 mL/min, 0.1 mL/min, 0.15 mL/min, 0.2 mL/min, 0.25 mL/min, 0.3 mL/min, 0.35 mL/min, 0.4 mL/min, 0.45 mL/min, or 0.5 mL/min. In an embodiment, the flow rate of the first and second polymer solutions through the needle are substantially the same. In an embodiment, the flow rate of the first and second polymer solutions through the needle are different.

In an embodiment, the voltage of the instrument is between 1 kV to 20 kV, or 1 to 15 kV, or 1 kV to 10 kV, or 5 kV to 10 kV. The voltage may be adjusted until a desired droplet rate is reached. In an embodiment, the droplet rate of the instrument is between 1 droplet/10 seconds to 50 droplets/10 seconds, or 1 droplet/10 seconds to 25 droplets/10 seconds.

In an embodiment, the number of non-particle debris on the surface of the cross-linking solution is determined. Particles that have fallen to the bottom of the cross-linking vessel may then be collected, e.g., by transferring cross-linking solution containing the particles to a separate container, leaving behind any non-particle debris on the solution surface in the original cross-linking vessel. The removed particles may then be allowed to settle, the cross-linking solution can be removed, and the particles may then be washed one or more times with a buffer (e.g., a HEPES buffer). In an embodiment, one or more aliquots of the resulting particle composition (e.g., preparation of particles) is inspected by microscopy to assess the quality of the composition, e.g., the number of particle defects and satellite particles.

In some embodiments, the cross-linking solution further comprises a process additive (e.g., a hydrophilic, non-ionic surfactant). A process additive may reduce surface tension of the cross-linking solution. Agents useful as the process additive in the present disclosure include polysorbate-type surfactants, copolymer of polyethyleneoxide (PEO) and polypropyleneoxide (PPO), poly(ethylene oxide)-poly(propylene oxide)-poly(ethylene oxide) (PEO-PPO-PEO) triblock copolymers, and non-ionic surfactants, such as Tween® 20, Tween® 80, Triton™ X-100, IGEPAL® CA-630, poloxamer 188, or poloxamer 407, or surfactants with substantially the same chemical and physical properties listed in the Exemplary Surfactant Table immediately below.

Exemplary Surfactant Table

| | | Approximate | |
|---|---|---|---|
| | | Average | |
| | | Molecular | |
| Brand or Generic | Commercial | Weight | Hydrophilicity |
| Name | Supplier | (g/mole) | HLB^{a} |
| Tween ® 20^{b} | Millipore Sigma | 1228 | 16.7 |
| Tween ® 80^{c} | Millipore Sigma | 1310 | 15 |
| Triton ™ X-100^{d} | Millipore Sigma | 625 | 13.4 |
| IGEPAL ® CA-630^{e} | Millipore Sigma | 603 | 13 |
| poloxamer 188^{f} | Millipore Sigma | 8400 | >24 |
| poloxamer 407^{g} | Millipore Sigma | 12,500 | 18-23 |

^{a}hydrophilic-lipophilic balance
^{b}Chemical names and synonyms: polyethylene glycol sorbitan monolaurate, polyoxyethylene (20) sorbitan monolaurate, polysorbate 20, polyoxyethylene 20 sorbitan monododecanoate
^{c}Chemical names and synonyms: polyethylene glycol sorbitan monooleate, polyoxyethylene (20) sorbitan monooleate, polysorbate 80, (x)-sorbitan mono-9-octadecenoate poly(oxy-1,2-ethanediyl)
^{d}Chemical names and synonyms: 4-(1,1,3,3-Tetramethylbutyl)phenyl-polyethylene glycol, t-octylphenoxypolyethoxyethanol, polyethylene glycol tert-octylphenyl ether; octylphenol ethoxylate, octylphenol ethylene oxide condensate
^{e}Chemical names and synonyms: octylphenoxypolyethoxyethanol, octylphenoxy poly(ethyleneoxy)ethanol, branched
^{f}Chemical name: Poly(ethylene glycol)-block-poly(propylene glycol)-block-poly(ethylene glycol)
^{g}Chemical name: Poly(ethylene glycol)-block-poly(propylene glycol)-block-poly(ethylene glycol)

In some embodiments, the process additive is a non-ionic surfactant. In an embodiment, the process additive comprises more than one surfactant, e.g., more than one hydrophilic surfactant. In some embodiments, the process additive does not contain Tween® 20 (polysorbate 20) or Triton™ X-100. In an embodiment, the process additive is IGEPAL® CA-630 (polyethylene glycol sorbitan monooleate). In some embodiments, the process additive is poloxamer 188.

In some embodiments, the process additive (e.g., surfactant) is present in the cross-linking solution at a concentration of at least 0.0001% or more. In some embodiments, the cross-linking solution comprises at least 0.001%, 0.01%, or 0.1% of the process additive. In some embodiments, the process additive is present at a concentration selected from about 0.001% to about 0.1%, about 0.005% to about 0.05%, about 0.005% to about 0.01%, and about 0.01% to about 0.5%. In an embodiment, the process additive is a surfactant and is present at a concentration that is below the critical micelle concentration for the surfactant.

In some embodiments, the cross-linking agent comprises divalent cations of a single type or a mixture of different types, e.g., one or more of Ba²⁺, Ca²⁺, Sr²⁺. In some embodiments, the cross-linking agent is BaCl₂, e.g., at a concentration of 1 mM to 100 mM or 7.5 mM to 20 mM. In some embodiments, the cross-linking agent is CaCl₂, e.g., at a concentration of 50 mM to 100 mM. In some embodiments, the cross-linking agent is SrCl₂, e.g., at a concentration of 37.5 mM to 100 mM. In some embodiments, the cross-linking agent is a mixture of BaCl₂ (e.g., 5 mM to 20 mM) and CaCl₂ (e.g., 37.5 mM to 12.5 mM) or a mixture of BaCl₂ (e.g., 5 mM to 20 mM) and SrCl₂ (e.g., 37.5 mM to 12.5 mM).

In some embodiments, the cross-linking agent is SrCl₂, and the process additive is Tween® 80 (or a surfactant with substantially the same chemical and physical properties listed in the Exemplary Surfactant Table) at a concentration of less than 0.1%, e.g., about 0.005% to 0.05%, about 0.005% to about 0.01%. In some embodiments, the concentration of SrCl₂ is about 50 mM. In some embodiments, the cross-linking agent is SrCl₂ and the process additive is poloxamer 188 at a concentration of 1%.

The type and concentration of buffer in the aqueous cross-linking solution is selected to maintain the solution pH at approximately neutral, e.g., from about 6.5 to about 7.5, about 7.0 to about 7.5, or about 7.0. In an embodiment, the buffer is compatible with a biological material to be encapsulated in the particle, e.g., cells. In some embodiments, the buffer in the aqueous cross-linking solution comprises HEPES (4-(2-hydroxyethyl)-1-piperazineethanesulfonic acid).

The osmolarity-adjusting agent in the aqueous cross-linking solution is selected to maintain the solution osmolarity at a value similar to the osmolarity of the polymer solution (which in some embodiments comprises a suspension of cells), e.g., an osmolarity that has a higher or lower variance of up to 20%, 10% or 5%. In some embodiments, the osmolarity agent is mannitol at a concentration of 0.1 M to 0.3 M.

In some embodiments, the cross-linking solution comprises 25 mM HEPES buffer, 20 mM BaCl₂, 0.2 M mannitol and 0.01% poloxamer 188.

In some embodiments, the cross-linking solution comprises 50 mM strontium chloride hexahydrate, 0.165 M mannitol, 25 mM HEPES and 0.01% of a surfactant with substantially the same chemical and physical properties listed in the Exemplary Surfactant Table for Tween 80.

In an embodiment, the process additive is poloxamer 188, which is present in the particle composition (e.g., preparation of particles) in a detectable amount after the wash steps. Poloxamer 188 may be detected by any technique known in the art, e.g., by partially or completely dissolving the particles in an aliquot of the composition by sodium sulfate precipitation and analyzing the supernatant by LC/MS.

Reduction in the surface tension of the cross-linking solution may be assessed by any method known in the art, for example, through the use of a contact angle goniometer or a tensiometer, e.g., via the du Nouy ring method (see, e.g., Davarci et al (2017) Food Hydrocolloids 62:119-127).

### EXEMPLARY ENUMERATED EMBODIMENTS

1. A polysaccharide polymer comprising:
(i) a crosslinking moiety; and
(ii) a compound of Formula (I):

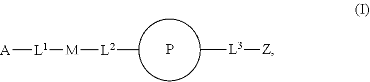

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ, —S(O)ₓN(R^{C})—, —P(R^{F})_{y}—, —Si(OR^{A})₂—, —Si(R^{G})(OR^{A}), —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R².
L² is a bond;
M is absent, alkyl, heteroalkyl, cycloalkyl, heterocyclyl, aryl, or heteroaryl, each of which is optionally substituted by one or more R³.
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶.
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, S(O)ₓN(R^{C1}(R^{D1}), —P(R^{F1}), cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

2. The polysaccharide polymer of embodiment 1, wherein the crosslinking moiety is covalently bound to a saccharide monomer within the polysaccharide polymer.

3. The polysaccharide polymer of embodiment 2, wherein the crosslinking moiety is bound to a carboxylate moiety within the saccharide monomer.

4. The polysaccharide polymer of any one of embodiments 1-3, wherein the crosslinking moiety comprises an alkyl, alkenyl, alkynyl, ester, ketone, amine, thiol, cycloalkyl, heterocyclyl, aryl, or heteroaryl group.

5. The polysaccharide polymer of any one of embodiments 1-4, wherein the crosslinking moiety is capable of reacting with a second crosslinking moiety upon activation, e.g., heat, acid, base, or a catalyst.

6. The polysaccharide polymer of any one of embodiments 1-5, wherein the crosslinking moiety is present on the polysaccharide polymer at a density of at least about 1%, e.g., 2%, 3%, 4%, 5%, 6%, 7%, 8%, 9%, 10%, or more, e.g., as determined by comparison to a reference standard.

7. The polysaccharide polymer of any one of embodiments 1-6, wherein the crosslinking moiety is present on the polysaccharide polymer at a density of between 1%-10%, e.g., 1%-8%, 1%-6%, or 1%-4%, e.g., as determined by comparison to a reference standard.

8. The polysaccharide polymer of any one of embodiments 1-7, wherein the polysaccharide polymer is selected from alginate, hyaluronate, and chitosan.

9. The polysaccharide polymer of any one of embodiments 1-8, wherein the polysaccharide polymer is alginate.

10. The polysaccharide polymer of embodiment 9, wherein the alginate is a high guluronic acid (G) alginate or a high mannuronic acid (M) alginate.

11. The polysaccharide polymer of any one of embodiments 1-10, wherein the crosslinking moiety has a structure of Formula (IV):

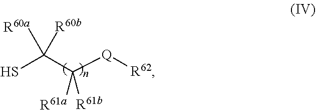

or a pharmaceutically acceptable salt or tautomer thereof, wherein:
Q is O, NR³³, or C(R^{34a})(R^{34b});
each of R³³, R^{34a}, R^{34b}, R^{60a}, R^{60b}, R^{6a}, R^{61b}, and R⁶² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

12. The polysaccharide polymer of embodiment 11, wherein the crosslinking moiety comprises a thiol moiety.

13. The polysaccharide polymer of any one of embodiments 1-10, wherein the crosslinking moiety has a structure of Formula (V):

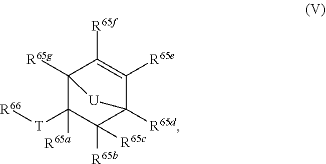

or a pharmaceutically acceptable salt or tautomer thereof, wherein:
each of T and U is independently O, NR³³, or C(R^{34a})(R^{34b});
each of R³³, R^{34a}, R^{34b}, R^{65a}, R^{65b}, R^{65c}, R^{65d}, R^{65e}, R^{65f}, R^{65g} and R⁶⁶ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

14. The polysaccharide polymer of embodiment 13, wherein the crosslinking moiety comprises a norbornenyl moiety.

15. The polysaccharide polymer of any one of embodiments 1-10, wherein the crosslinking moiety has a structure of Formula (VI):

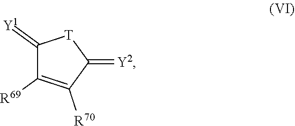

or a pharmaceutically acceptable salt or tautomer thereof, wherein:
each of T, Y¹, and Y² is independently O, NR³³, or C(R^{34a})(R^{34b});
each of R³³, R^{34a}, R^{34b}, R⁶⁹, and R⁷⁰ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

16. The polysaccharide polymer of embodiment 15, wherein the crosslinking moiety comprises a maleimide moiety.

17. The polysaccharide polymer of any one of embodiments 1-10, wherein the crosslinking moiety has a structure of Formula (VII):

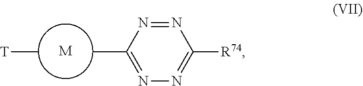

or a pharmaceutically acceptable salt or tautomer thereof, wherein:
T is O, NR³³, or C(R^{34a})(R^{34b});
Ring M is cycloalkyl, heterocyclyl, aryl, heteroaryl, each of which is optionally substituted with 1-6 R⁷;
each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

18. The polysaccharide polymer of embodiment 17, wherein the crosslinking moiety comprises a tetrazinyl moiety.

19. The polysaccharide polymer of any one of embodiments 1-18, wherein the crosslinking moiety has a structure selected from Table 4, or a pharmaceutically acceptable salt thereof.

20. The polysaccharide polymer of any one of embodiments 1-19, wherein the polysaccharide polymer comprises one of a compound of Formula (IV), (V), (VI), or (VII), or a pharmaceutically acceptable salt thereof.

21. The polysaccharide polymer of any one of embodiments 1-20, wherein the polysaccharide polymer comprises two of a compound of Formula (IV), (V), (VI), or (VII), or a pharmaceutically acceptable salt thereof.

22. The polysaccharide polymer of any one of embodiments 1-21, wherein the compound of Formula (I) has a structure selected from Table 3, or a pharmaceutically acceptable salt thereof.

23. The polysaccharide polymer of any one of embodiment 1-22, wherein the compound of Formula (I) is selected from Compound 100, Compound 101, Compound 110, Compound 112, Compound 113, Compound 114, Compound 122, and Compound 123, or a pharmaceutically acceptable salt thereof.

24. The polysaccharide polymer of any one of embodiment 1-23, wherein the compound of Formula (I) is Compound 101 or a pharmaceutically acceptable salt thereof.

25. The polysaccharide polymer of any one of embodiment 1-24, wherein the polysaccharide polymer is alginate, the crosslinking moiety is selected from a compound listed in Table 4 or a pharmaceutically acceptable salt thereof, and the compound of Formula (I) is Compound 101 or a pharmaceutically acceptable salt thereof.

26. A composition comprising a polysaccharide polymer of any one of embodiments 1-25.

27. A hydrogel capsule comprising a polysaccharide polymer of any one of embodiments 1-25.

28. The hydrogel capsule of embodiment 27, wherein the hydrogel capsule comprises a single compartment comprising the polysaccharide polymer (e.g., a polysaccharide polymer described herein).

29. The hydrogel capsule of embodiment 27, wherein the hydrogel capsule comprises a plurality of compartments, wherein one of the compartments comprises the polysaccharide polymer (e.g., a polysaccharide polymer described herein).

30. The hydrogel capsule of embodiment 29, wherein the hydrogel capsule comprises an inner compartment and an outer compartment.

31. The hydrogel capsule of embodiment 30, wherein:
the inner compartment comprises a first polysaccharide polymer comprising the crosslinking moiety;
the outer compartment comprises a second polysaccharide polymer comprising the crosslinking moiety.

32. A hydrogel capsule comprising:
(i) an inner compartment comprising a first polysaccharide polymer comprising a compound of Formula (I):

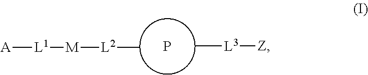

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O_, —S—, —S(O)ₓ, —OS(O)ₓ, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D} taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4; and
(ii) an outer compartment comprising a second polysaccharide polymer comprising a crosslinking moiety.

33. The hydrogel capsule of embodiment 32, wherein the polysaccharide polymer (e.g., the first polysaccharide polymer and/or the second polysaccharide polymer) is selected from alginate, hyaluronate, and chitosan.

34. The hydrogel capsule of any one of embodiment 32-33, wherein the polysaccharide polymer (e.g., the first polysaccharide polymer and/or the second polysaccharide polymer) is alginate.

35. The hydrogel capsule of any one of embodiments 32-34, wherein the first polysaccharide polymer is alginate.

36. The hydrogel capsule of any one of embodiments 32-35, wherein the second polysaccharide polymer is alginate.

37. The hydrogel capsule of any one of embodiments 32-36, wherein the alginate is a high guluronic acid (G) alginate or a high mannuronic acid (M) alginate.

38. The hydrogel capsule of any one of embodiments 32-37, wherein the crosslinking moiety has a structure of Formula (IV):

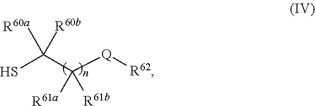

or a pharmaceutically acceptable salt or tautomer thereof, wherein:
Q is O, NR³³, or C(R^{34a})(R^{34b});
each of R³³, R^{34a}, R^{34b}, R^{60a}, R^{60b}, R^{61a}, R^{61b}, and R⁶² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

39. The hydrogel capsule of any one of embodiments 32-37, wherein the crosslinking moiety has a structure of Formula (V):

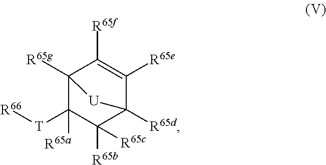

or a pharmaceutically acceptable salt or tautomer thereof, wherein:
each of T and U is independently O, NR³³, or C(R^{34a})(R^{34b});
each of R³³, R^{34a}, R^{34b}, R^{65a}, R^{65b}, R^{65c}, R^{65d}, R^{65e}, R^{65f}, R^{65g} and R⁶⁶ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

40. The hydrogel capsule of any one of embodiments 32-37, wherein the crosslinking moiety has a structure of Formula (VI):

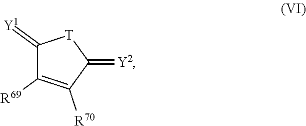

or a pharmaceutically acceptable salt or tautomer thereof, wherein:
each of T, Y¹, and Y² is independently O, NR³³, or C(R^{34a})(R^{34b});
each of R³³, R^{34a}, R^{34b}, R⁶⁹, and R⁷⁰ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

41. The hydrogel capsule of any one of embodiments 32-37, wherein the crosslinking moiety has a structure of Formula (VII):

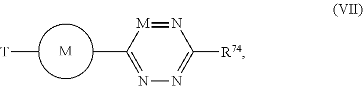

or a pharmaceutically acceptable salt or tautomer thereof, wherein:
T is O, NR³³, or C(R^{34a})(R^{34b});
Ring M is cycloalkyl, heterocyclyl, aryl, heteroaryl, each of which is optionally substituted with 1-6 R⁷;
each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

42. The hydrogel capsule of any one of embodiments 38-41, wherein the compound of Formula (IV), (V), (VI), or (VII) is selected from a compound in Table 4 or a pharmaceutically acceptable salt thereof.

43. The hydrogel capsule of any one of embodiments 32-42, wherein the compound of Formula (I) has a structure selected from Table 3, or a pharmaceutically acceptable salt thereof.

44. The hydrogel capsule of any one of embodiments 32-43, wherein the compound of Formula (I) is selected from Compound 100, Compound 101, Compound 110, Compound 112, Compound 113, Compound 114, Compound 122, and Compound 123, or a pharmaceutically acceptable salt thereof.

45. The hydrogel capsule of any one of embodiments 32-44, wherein the compound of Formula (I) is Compound 101 or a pharmaceutically acceptable salt thereof.

46. The hydrogel capsule of any one of embodiments 32-45, wherein the hydrogel capsule has a diameter of between 0.1 mm to 5 mm

47. The hydrogel capsule of any one of embodiments 32-46, wherein the hydrogel capsule has a diameter of between 1 mm to 5 mm.

48. The hydrogel capsule of any one of embodiments 32-47, wherein the hydrogel capsule has a diameter of between 1 mm to 2.5 mm.

49. The hydrogel capsule of any one of embodiments 32-48, wherein the hydrogel capsule encapsulates a cell.

50. The hydrogel capsule of embodiment 49, wherein the cell produces a therapeutic agent.

51. The hydrogel capsule of embodiment 50, wherein the therapeutic agent is a protein, e.g., a hormone, a blood clotting factor, an antibody, or an enzyme.

52. The hydrogel capsule of any one of embodiments 32-51, wherein the hydrogel capsule is formulated for implantation into a subject (e.g., into the intraperitoneal (IP) space, the peritoneal cavity, the omentum, the lesser sac, the subcutaneous fat).

53. The hydrogel capsule of any one of embodiments 32-52, wherein the implantable element is formulated for implantation into the IP space of a subject.

54. A composition comprising a hydrogel capsule of any one of embodiments 32-53.

55. A method of producing a hydrogel capsule comprising a polysaccharide polymer of any one of embodiments 1-24.

56. A method of increasing the stability of a hydrogel capsule comprising polysaccharide polymers, wherein the method comprises providing a means of both ionically crosslinking the polysaccharide polymers and covalently crosslinking the polysaccharide polymers.

57. The method of embodiment 56, wherein the means of ionically crosslinking the polysaccharide polymers comprises use of a divalent cation (e.g., Ba²⁺, Ca²⁺, Sr²⁺).

58. The method of any one of embodiments 56-57, wherein the means of covalently crosslinking the polysaccharide polymers comprises use of a crosslinking moiety.

59. The polysaccharide polymer of any one of embodiments 1-25, wherein the polysaccharide retains additional carboxylic acid groups following addition of the clickable crosslinker.

60. The polysaccharide polymer of any one of embodiments 1-25, or 59, wherein the polysaccharide is not reduced (e.g., not treated with a reducing agent) prior to modification with a clickable crosslinker.

61. The polysaccharide polymer of any one of embodiments 1-25, or 59, wherein the polysaccharide polymer is not oxidized (e.g., not treated with an oxidizing agent) prior to modification with a clickable crosslinker.

62. The polysaccharide polymer of any one of embodiments 1-25, or 59-61, wherein the clickable crosslinker is not hydrolysable.

63. The polysaccharide polymer of any one of embodiments 1-25, or 59-62, wherein the compound of Formula (I) is not hydrolysable.

64. The polysaccharide polymer of any one of embodiments 1-25, or 59-63, wherein the clickable crosslinker is not a thiol or vinyl sulfone.

65. A method of treating a disease, disorder or condition in a subject, wherein the method comprises administering to the subject a hydrogel capsule of any one of embodiments 32-53.

66. The method of embodiment 65, wherein the disease, disorder, or condition is diabetes (e.g., Type 1 diabetes).

67. The method of embodiment 65, wherein the disease, disorder, or condition is not diabetes (e.g., Type 1 diabetes).

68. The method of any one of embodiments 65-67, wherein the subject is human.

### EXAMPLES

In order that the invention described herein may be more fully understood, the following examples are set forth. The synthetic and biological examples described in this application are offered to illustrate the compounds, compositions, devices, and methods provided herein and are not to be construed in any way as limiting their scope.

The compounds, modified polymers, implantable elements, and compositions thereof provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Exemplary compounds, modified polymers, implantable elements, and compositions of the invention may be prepared using any of the strategies described below.

### Example 1: Synthesis of Sodium Alginate Modified with an Exemplary Thiol and Compound of Formula (I)

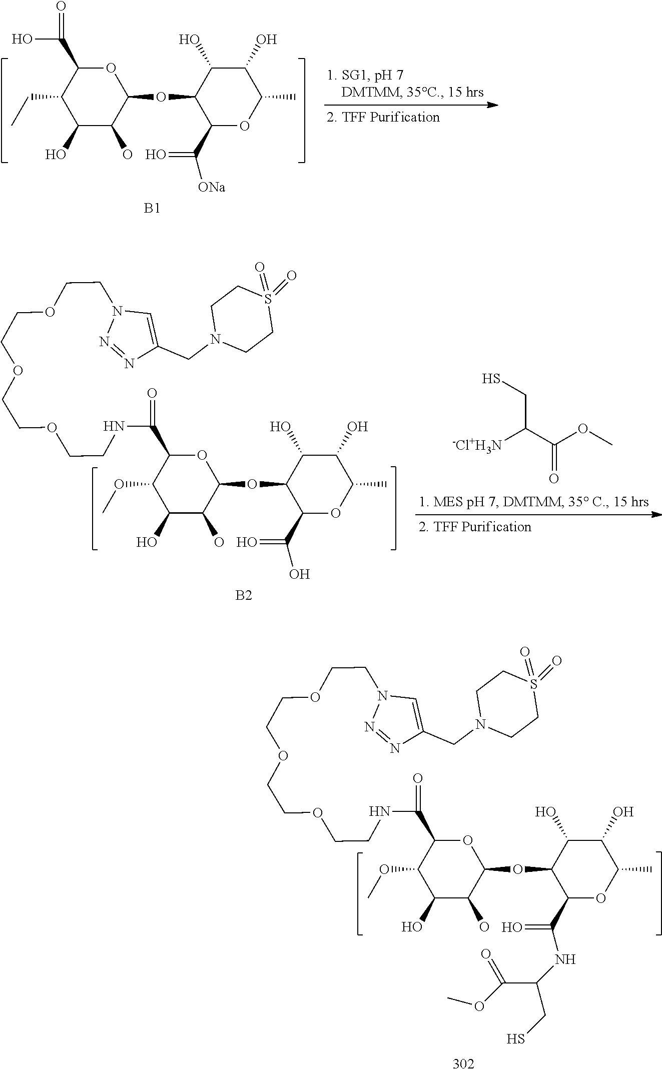

In this example, alginate polymers comprising a thiol crosslinker agent and a compound of Formula (I) are synthesized. Nova Matrix PRONOVA™ UP LG20 (300 g; 1.25% w/w in water, 3.75 g sodium alginate) is weighed into a 400 mL EasyMax reactor equipped with overhead stirring. In a separate 150 mL sterile container is massed 4-((1-(2-(2-(2-(2-aminoethoxy)ethoxy)ethoxy)ethyl)-1H-1,2,3-triazol-4-yl)methyl)thiomorpholine 1,1-dioxide (5.77 g, 14.74 mmol) along with endotoxin free water (20 g) and mixed on a shaker at 300 rpm until fully dissolved. Once dissolved, the pH is adjusted to pH 7.0 with 6N and 1N hydrochloric acid and charged to the EasyMax reactor. Agitation is set at 300 rpm, and the batch temperature is adjusted to 25° C. In a separate 150 mL container is massed 4-(4,6-dimethoxy-1,3,5-triazin-2-yl)-4-methylmorpholinium chloride (3.88 g, 14.02 mmol) along with endotoxin free water (45 g) and mixed by hand until fully dissolved. The solution is added to the EasyMax reactor over a period of two minutes. Once the addition is complete, the batch is heated to 35° C. in 1 hour, held at 35′C for 15 hours before cooling to 25° C. Once the reaction is complete, the batch is filtered through a pad of cyano-silica prior to purification via tangential flow filtration (10 kDa molecular weight cutoff). The solution is first purified against 10 volume exchanges with normal saline followed by 10 volume exchanges with endotoxin free water.

After purification, the solution is concentrated to a refractive index value of 1.3360 and charged back into the 400 mL EasyMax reactor. Agitation is set to 300 rpm and the batch temperature is adjusted to 25° C. To add on the thiol crosslinker agent, exemplary reaction conditions follow. In a separate sterile container, mecysteine hydrochloride (0.91 g, 5.28 mmol) is weighed and dissolved in 1M MES pH 7.0 buffer (7.5 mL) and then charged to the reactor. 4-(4,6-dimethoxy-1,3,5-triazin-2-yl)-4-methylmorpholinium chloride (1.61 g, 5.82 mmol) is weighed in a sterile container and dissolved in 1M MES pH 7.0 buffer (10 mL) and then added to the reactor over two minutes. Once the charge is complete, the reaction mixture is heated to 35° C. in 1 hour, held at 35° C. for 15 hours before cooling to 25° C. The reaction mixture is purified via tangential flow filtration (10 kDa molecular weight cutoff) against 10 volumes exchanges with saline. After purification, the solution is concentrated to a refractive index value of 1.3380.

### Example 2: Synthesis of Sodium Alginate Modified with an Exemplary Maleimide

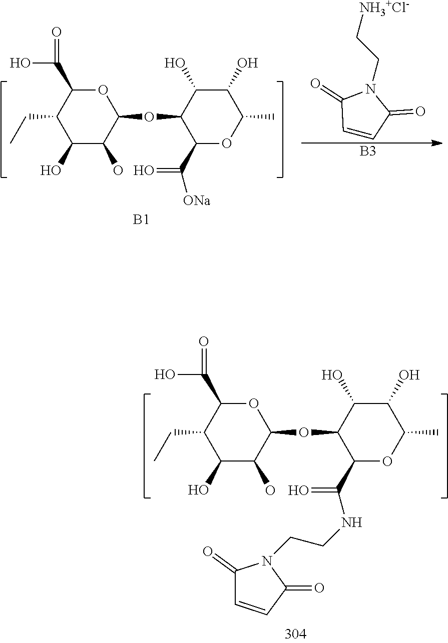

In this example, alginate polymers comprising a maleimide crosslinker agent may be synthesized according to the exemplary procedures outlined below. Nova Matrix PRONOVA™ UP LG20 (300 g; 1.25% w/w in water, 3.75 g sodium alginate) is weighed into a 400 mL EasyMax reactor equipped with overhead stirring. In a separate sterile container, 1-(2-aminoethyl)-maleimide hydrochloride (0.93 g, 5.28 mmol) is weighed and dissolved in 1M MES pH 7.0 buffer (7.5 mL) and then charged to the reactor. 4-(4,6-dimethoxy-1,3,5-triazin-2-yl)-4-methylmorpholinium chloride (1.61 g, 5.82 mmol) is weighed in a sterile container and dissolved in 1M MES pH 7.0 buffer (10 mL) and then added to the reactor over two minutes. Once the charge is complete, the reaction mixture is heated to 35° C. in 1 hour, held at 35° C. for 15 hours before cooling to 25° C. The reaction mixture is purified via tangential flow filtration (10 kDa molecular weight cutoff) against 10 volumes exchanges with saline. After purification, the solution is concentrated to a refractive index value of 1.3380.

### Example 3: Synthesis of Dual Crosslinked Alginate Polymers Through a Michael Addition Reaction

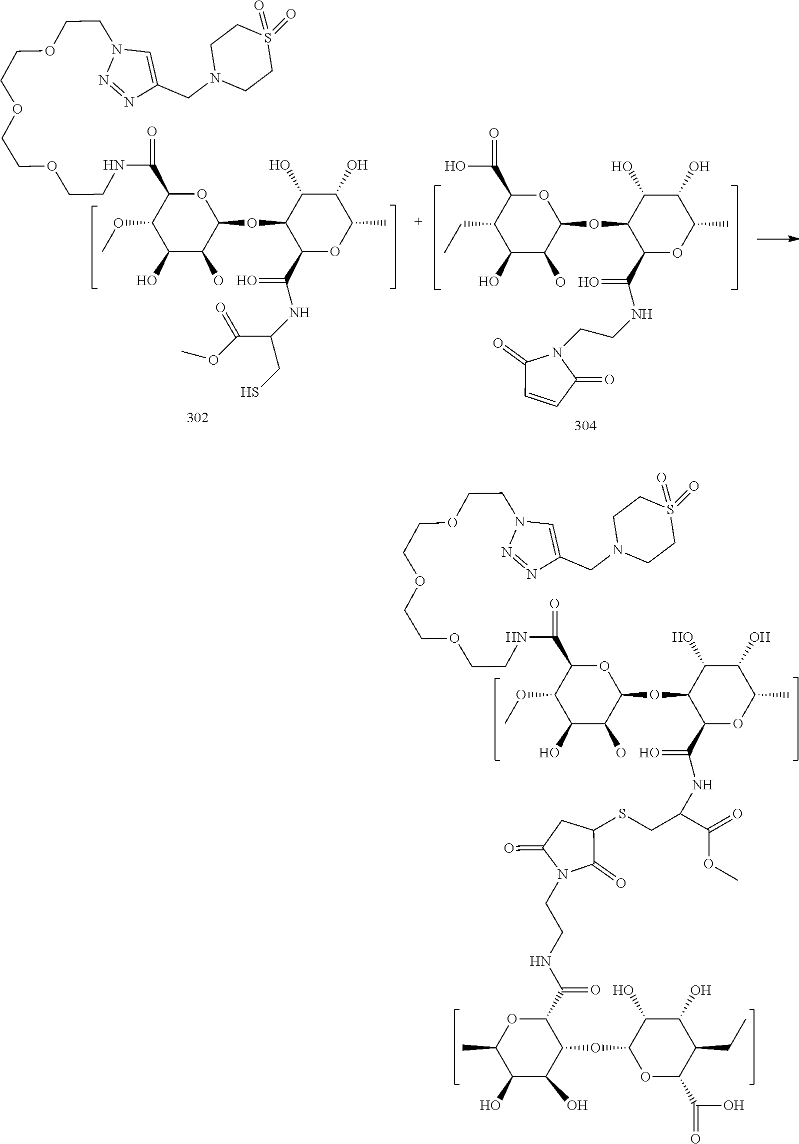

To form the covalently crosslinked alginates, the thiol crosslinker and the maleimide crosslinker may be coupled together, according to the exemplary protocol outlined herein: an alginate polymer comprising Compound 302 and an alginate polymer comprising Compound 304 (1:3 molar ratio) are dissolved in 1M MES buffer and allowed to incubate at 20-30° C. for 4-12 h. The reaction mixture is purified via tangential flow filtration (10 kDa molecular weight cutoff) against 10 volumes exchanges with saline. After purification, the solution is concentrated to a refractive index value of 1.3380.

### Example 4: Synthesis of Sodium Alginate Modified with an Exemplary Tetrazine and Compound of Formula (I)

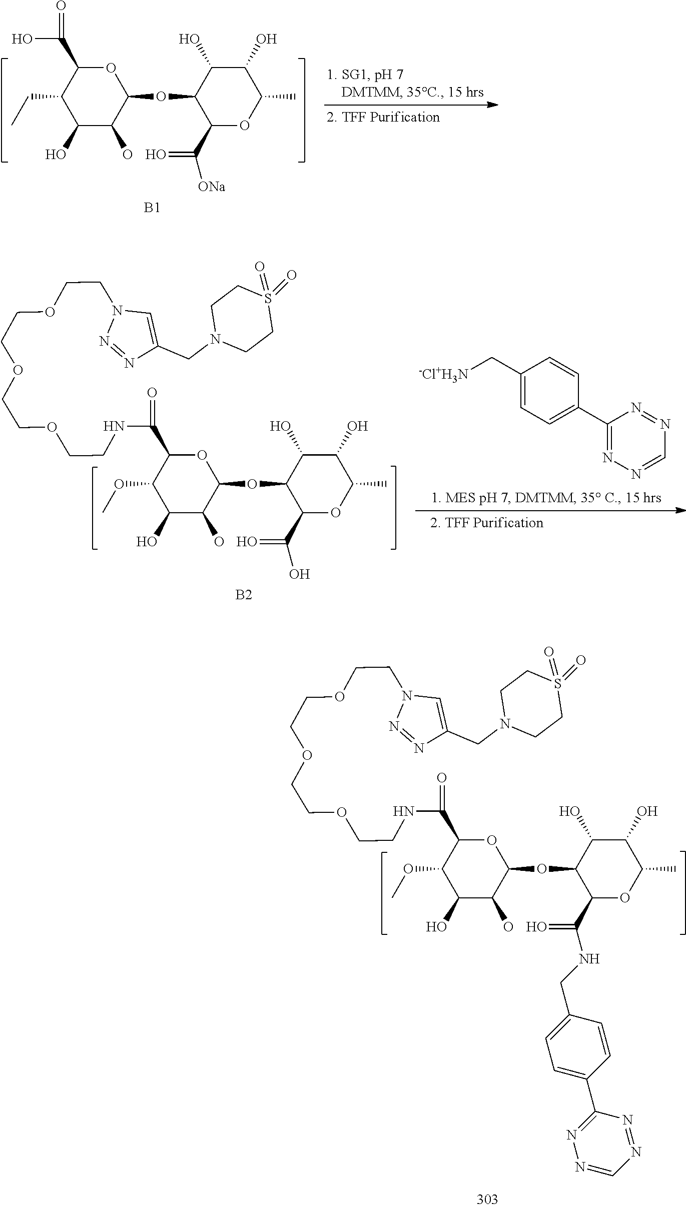

In this example, alginate polymers comprising an thiol crosslinker agent and a compound of Formula (I) are synthesized. Nova Matrix PRONOVA™ UP LG20 (300 g; 1.25% w/w in water, 3.75 g sodium alginate) is weighed into a 400 mL EasyMax reactor equipped with overhead stirring. In a separate 150 mL sterile container is massed 4-((1-(2-(2-(2-(2-aminoethoxy)ethoxy)ethoxy)ethyl)-1H-1,2,3-triazol-4-yl)methyl)thiomorpholine 1,1-dioxide (5.77 g, 14.74 mmol) along with endotoxin free water (20 g) and mixed on a shaker at 300 rpm until fully dissolved. Once dissolved, the pH is adjusted to pH 7.0 with 6N and 1N hydrochloric acid and charged to the EasyMax reactor. Agitation is set at 300 rpm, and the batch temperature is adjusted to 25° C. In a separate 150 mL container is massed 4-(4,6-dimethoxy-1,3,5-triazin-2-yl)-4-methylmorpholinium chloride (3.88 g, 14.02 mmol) along with endotoxin free water (45 g) and mixed by hand until fully dissolved. The solution is added to the EasyMax reactor over a period of two minutes. Once the addition is complete, the batch is heated to 35° C. in 1 hour, held at 35° C. for 15 hours before cooling to 25° C. Once the reaction is complete, the batch is filtered through a pad of cyano-silica prior to purification via tangential flow filtration (10 kDa molecular weight cutoff). The solution is first purified against 10 volume exchanges with normal saline followed by 10 volume exchanges with endotoxin free water.

After purification, the solution is concentrated to a refractive index value of 1.3360 and charged back into the 400 mL EasyMax reactor. Agitation is set to 300 rpm and the batch temperature is adjusted to 25° C. To add on the thiol crosslinker agent, exemplary reaction conditions follow. In a separate sterile container, 1-[4-(1,2,4,5-Tetrazin-3-yl)phenyl]methanamine (1.18 g, 5.28 mmol) is weighed and dissolved in 1M MES pH 7.0 buffer (7.5 mL) and then charged to the reactor. 4-(4,6-dimethoxy-1,3,5-triazin-2-yl)-4-methylmorpholinium chloride (1.61 g, 5.82 mmol) is weighed in a sterile container and dissolved in 1M MES pH 7.0 buffer (10 mL) and then added to the reactor over two minutes. Once the charge is complete, the reaction mixture is heated to 35° C. in 1 hour, held at 35° C. for 15 hours before cooling to 25° C. The reaction mixture is purified via tangential flow filtration (10 kDa molecular weight cutoff) against 10 volumes exchanges with saline. After purification, the solution is concentrated to a refractive index value of 1.3380.

### Example 5: Synthesis of Sodium Alginate Modified with an Exemplary Norbornene

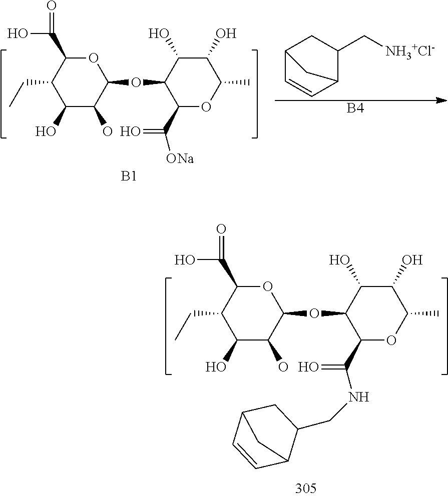

In this example, alginate polymers comprising a maleimide crosslinker agent may be synthesized according the exemplary procedures outlined below. Nova Matrix PRONOVA™ UP LG20 (300 g; 1.25% w/w in water, 3.75 g sodium alginate) is weighed into a 400 mL EasyMax reactor equipped with overhead stirring. In a separate sterile container, 5-norbornene-2-methylamine (0.84 g, 5.28 mmol) is weighed and dissolved in 1M MES pH 7.0 buffer (7.5 mL) and then charged to the reactor. 4-(4,6-dimethoxy-1,3,5-triazin-2-yl)-4-methylmorpholinium chloride (1.61 g, 5.82 mmol) is weighed in a sterile container and dissolved in 1M MES pH 7.0 buffer (10 mL) and then added to the reactor over two minutes. Once the charge is complete, the reaction mixture is heated to 35° C. in 1 hour, held at 35° C. for 15 hours before cooling to 25° C. The reaction mixture is purified via tangential flow filtration (10 kDa molecular weight cutoff) against 10 volumes exchanges with saline. After purification, the solution is concentrated to a refractive index value of 1.3380.

### Example 6: Synthesis of Dual Crosslinked Alginate Polymers by Inverse Electron Demand Diels Alder Reaction

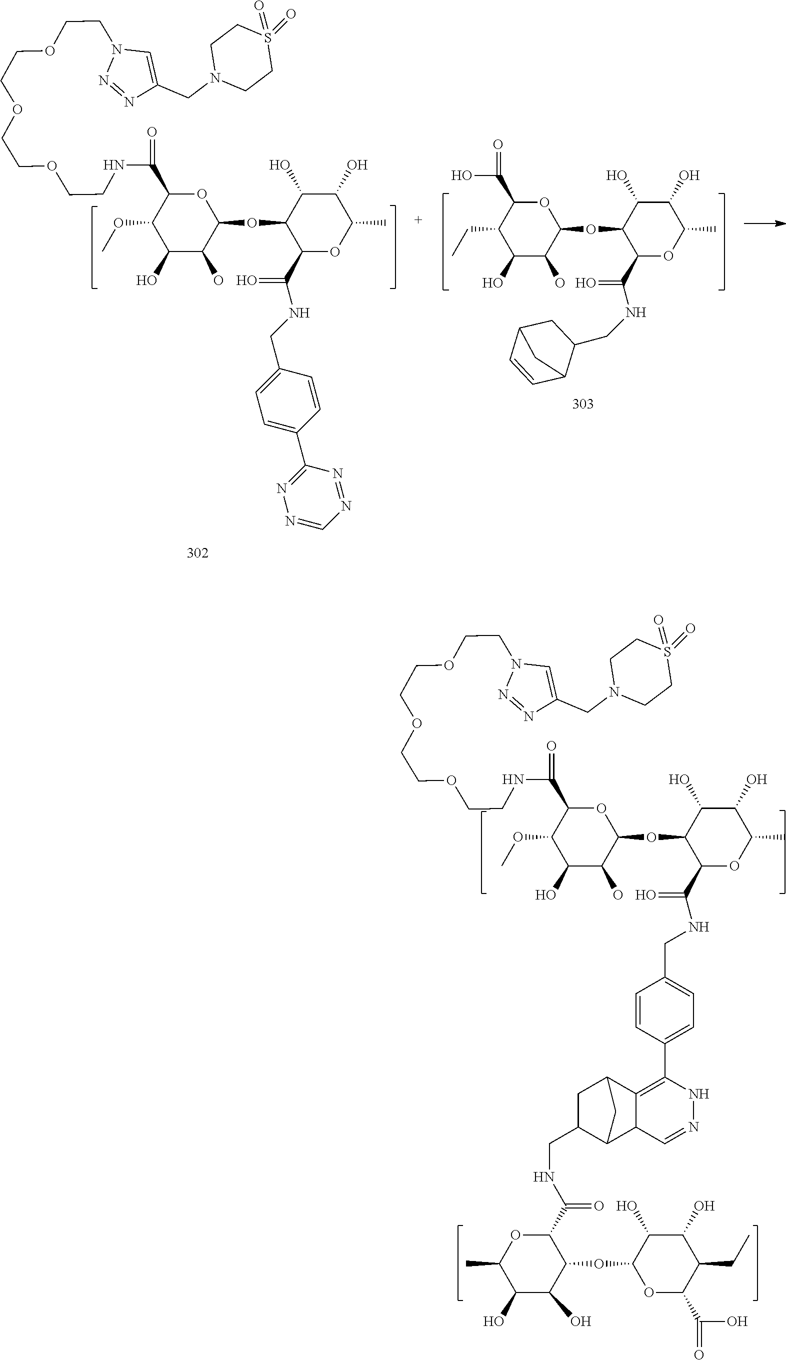

To form the covalently crosslinked alginates, the thiol crosslinker and the maleimide crosslinker may be coupled together, according to the exemplary protocol outlined herein: an alginate polymer comprising Compound 303 and an alginate polymer comprising Compound 305 are dissolved in 1M MES buffer and allowed to incubate at 20-30° C. for 4-12 h. The reaction mixture is purified via tangential flow filtration (10 kDa molecular weight cutoff) against 10 volumes exchanges with saline. After purification, the solution is concentrated to a refractive index value of 1.3380.

### Example 7: Synthesis of Dual Crosslinked Alginate Polymers by Thiol-Ene Photoclick Reaction

To form the covalently crosslinked alginates, the thiol crosslinker and the norbornene crosslinker may be coupled together, according to the exemplary protocol outlined herein: an alginate polymer comprising Compound 301 or 300 and an alginate polymer comprising Compound 305 (1:2 molar ratio), along with lithium phenyl-2,4,6-trimethylbenzoylphosphinate (LAP) as a photoinitiator, are dissolved in 1M MES buffer and irradiated with UV light for 0.5 h with stirring. The reaction mixture is purified via tangential flow filtration (10 kDa molecular weight cutoff) against 10 volumes exchanges with saline. After purification, the solution is concentrated to a refractive index value of 1.3380.

### Example 8: Synthesis of Exemplary Dual Crosslinked Alginate Hydrogel Capsules

Prior to fabrication of one-compartment or two-compartment alginate hydrogel capsules, buffers and alginate solutions were sterilized by filtration through a 0.2-μm filter using aseptic processes. To prepare particles configured as two-compartment hydrogel capsules of about 1.5 mm diameter, an electrostatic droplet generator was set up as follows: an ES series 0-100-kV, 20-watt high-voltage power generator (EQ series, Matsusada, NC, USA) was connected to the top and bottom of a coaxial needle (inner lumen of 22G, outer lumen of 18G, Paragon). The inner lumen was attached to a first 5-ml Luer-lock syringe (BD, NJ, USA), which was connected to a syringe pump (Pump 11 Pico Plus, Harvard Apparatus, Holliston, MA, USA) that was oriented vertically. The outer lumen was connected via a luer coupling to a second 5-ml Luer-lock syringe which was connected to a second syringe pump (Pump 11 Pico Plus) that was oriented horizontally. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump were 12.06 mm diameter and the flow rates of each pump were adjusted to achieve various test flow rates in the Examples below, but keeping the total flow rate set at 10 ml/h.

For fabrication of both the two-compartment and one-compartment dual crosslinked alginate hydrogel capsules, after extrusion of the desired volumes of alginate solutions, the alginate droplets were ionically crosslinked for five minutes in a crosslinking solution which contained 25 mM HEPES buffer, 20 mM BaCl₂, and 0.2M mannitol. In some experiments, the crosslinking solution also contained 0.01% of poloxamer 188. Capsules that had fallen to the bottom of the crosslinking vessel were collected by pipetting into a conical tube. After the capsules settled in the tube, the crosslinking buffer was removed, and capsules were washed. Capsules were washed four times with HEPES buffer and resuspended in

In some experiments, the quality of capsules in a composition of two-compartment or one-compartment capsules was examined. An aliquot containing at least 200 capsules was taken from the composition and transferred to a well plate and the entire aliquot examined by optical microscopy for quality by counting the number of spherical capsules out of the total. In some experiments, the mechanical strength of capsules in a composition of two-compartment capsules was examined using a texture analyzer to determine the initial fracture force as described herein above.

### Example 9: Preparation of Two-Compartment Hydrogels Comprising Modified Polysaccharide Polymers

Using the method of Example 8, two-compartment hydrogels may be synthesized from the alginate polymers described in Examples 3, 6, and 7. As a non-limiting example, a two-compartment hydrogel comprising both inner and outer compartments of the dual crosslinked alginate of Example 3 may be synthesized according to this method. As another example, a two-compartment hydrogel comprising an inner compartment of the dual crosslinked alginate of Example 6 and an outer compartment comprising the dual crosslinked alginate of Example 7 may be synthesized.

### Example 10: Synthesis of Exemplary Dual Crosslinked Alginate Hydrogel Capsules

The fracture or mechanical strength of a particle (e.g., a hydrogel capsule) may be determined after manufacture but before implantation by performing a fracture test using a texture analyzer. In an embodiment, mechanical testing of hydrogel capsules is performed on a TA.XT plus Texture Analyzer (Stable Micro Systems, Surrey, United Kingdom) using a 5 mm probe attached to a 5 kg load cell. Individual capsules are placed on a platform and are compressed from above by the probe at a fixed rate of 0.5 mm/sec. Contact between the probe and capsule is detected when a repulsive force of 1 g is measured. The probe continues to travel 90% of the distance between the contact height of the probe and the platform, compressing the capsule to the point of bursting. The resistance to the compressive force of the probe is measured and can be plotted as a function of probe travel (force v. displacement curve). Typically, before a capsule bursts completely, it will fracture slightly and the force exerted against the probe will decrease a small amount. An analysis macro can be programmed to detect the first time a decrease of 0.25-0.5 g occurs in the force v. displacement curve. The force applied by the probe when this occurs is termed the initial fracture force. In an embodiment, the fracture force for a capsule preparation manufactured using an apparatus described herein is the average of the initial fracture force for at least 10, 20, 30 or 40 capsules.

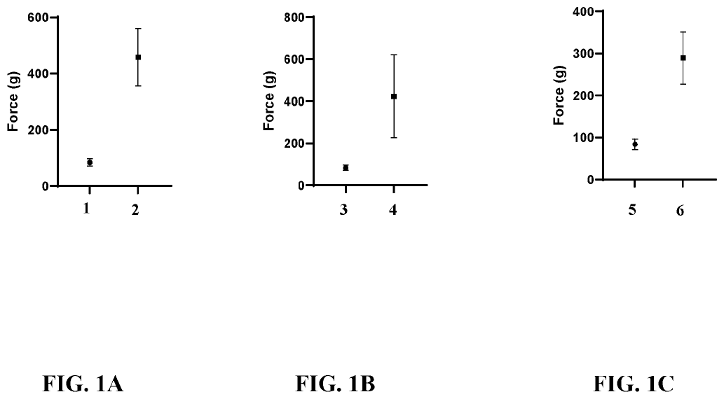

In this example, three exemplary alginate hydrogel capsule formulations were prepared according to the protocols outlined in Examples 3, 6, and 7 and their fracture strength was analyzed. The hydrogel capsule architectures are as described in Table 6. As show in FIGS. 1A-1C, the dual crosslinked alginate hydrogels exhibit increased average fracture strength compared to a hydrogel consisting of only ionic (e.g., Ba²⁺ mediated) crosslinking. In FIGS. 1A-1C, (1), (3) and (5) refer to ionically crosslinked alginate hydrogels, while (2), (4), and (6) refer to hydrogel capsules 1, 2, and 3 as shown in Table 6.

TABLE 6
Exemplary Two-Compartment Hydrogel Architectures

| | Hydrogel | Hydrogel | Hydrogel |
|---|---|---|---|
| | Capsule 1 | Capsule 2 | Capsule 3 |
| Identity of | Compound 300 + | Compound 300 + | Compound 301 + |
| inner and | Compound 301 | Compound 304 | Compound 309 |
| outer | | | |
| compartments | | | |
| Ref. | Example 7 | Example 3 | Example 6 |

### Example 11: Encapsulation of Mammalian Cells in Hydrogels Comprising Modified Polysaccharide Polymers

Exemplary mammalian cells may be encapsulated in the hydrogels described in Example 10. Cells may be added to the inner and/or outer layers of the two-compartment hydrogel (e.g., at densities of 5-8×10⁶ cells mL⁻¹).

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A polysaccharide polymer comprising:
(i) a crosslinking moiety; and
(ii) a compound of Formula (I): 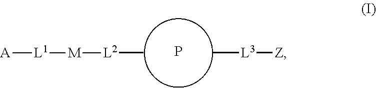
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

2. The polysaccharide polymer of claim 1, wherein the crosslinking moiety is covalently bound to a saccharide monomer within the polysaccharide polymer.

3. The polysaccharide polymer of claim 2, wherein the crosslinking moiety is bound to a carboxylate moiety within the saccharide monomer.

4. The polysaccharide polymer of claim 1, wherein the crosslinking moiety comprises an alkyl, alkenyl, alkynyl, ester, ketone, amine, thiol, cycloalkyl, heterocyclyl, aryl, or heteroaryl group.

5. The polysaccharide polymer of claim 1, wherein the crosslinking moiety is capable of reacting with a second crosslinking moiety upon activation, e.g., heat, acid, base, or a catalyst.

6. The polysaccharide polymer of claim 1, wherein the crosslinking moiety is present on the polysaccharide polymer at a density of at least about 1%, e.g., 2%, 3%, 4%, 5%, 6%, 7%, 8%, 9%, 10^{%}, or more, e.g., as determined by comparison to a reference standard.

7. The polysaccharide polymer of claim 1, wherein the crosslinking moiety is present on the polysaccharide polymer at a density of between 1%-10%, e.g., 1%-8%, 1%-6%, or 1%-4%, e.g., as determined by comparison to a reference standard.

8. The polysaccharide polymer of claim 1, wherein the polysaccharide polymer is selected from alginate, hyaluronate, and chitosan.

9. The polysaccharide polymer of claim 1, wherein the polysaccharide polymer is alginate.

10. The polysaccharide polymer of claim 9, wherein the alginate is a high guluronic acid (G) alginate or a high mannuronic acid (M) alginate.

11. The polysaccharide polymer of claim 1, wherein the crosslinking moiety has a structure of Formula (IV): 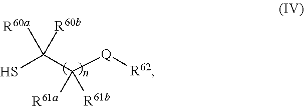
or a pharmaceutically acceptable salt or tautomer thereof, wherein:
Q is O, NR³³, or C(R^{34a})(R^{34b});
each of R³³, R^{34a}, R^{34b}, R^{60a}, R^{60b}, R^{61a}, R^{61b}, and R⁶² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

12. The polysaccharide polymer of claim 11, wherein the crosslinking moiety comprises a thiol moiety.

13. The polysaccharide polymer of claim 1, wherein the crosslinking moiety has a structure of Formula (V): 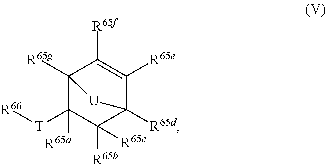
or a pharmaceutically acceptable salt or tautomer thereof, wherein:
each of T and U is independently O, NR³³, or C(R^{34a})(R^{34b});
each of R³³, R^{34a}, R^{34b}, R^{65a}, R^{65b}, R^{65c}, R^{65d}, R^{65e}, R^{65c}, R^{65g} and R⁶⁶ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

14. The polysaccharide polymer of claim 13, wherein the crosslinking moiety comprises a norbornenyl moiety.

15. The polysaccharide polymer of claim 1, wherein the crosslinking moiety has a structure of Formula (VI): 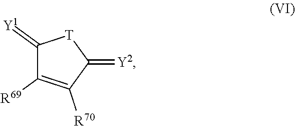
or a pharmaceutically acceptable salt or tautomer thereof, wherein:
each of T, Y¹, and Y² is independently O, NR³³, or C(R^{34a})(R^{34b});
each of R³³, R^{34a}, R^{34b}, R⁶⁹, and R⁷⁰ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

16. The polysaccharide polymer of claim 15, wherein the crosslinking moiety comprises a maleimide moiety.

17. The polysaccharide polymer of claim 1, wherein the crosslinking moiety has a structure of Formula (VII): 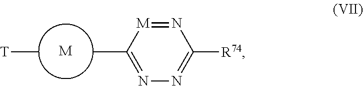
or a pharmaceutically acceptable salt or tautomer thereof, wherein:
T is O, NR³³, or C(R^{34a})(R^{34b});
Ring M is cycloalkyl, heterocyclyl, aryl, heteroaryl, each of which is optionally substituted with 1-6 R⁷;
each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

18. The polysaccharide polymer of claim 17, wherein the crosslinking moiety comprises a tetrazinyl moiety.

19. The polysaccharide polymer of claim 1, wherein the crosslinking moiety has a structure selected from Table 4, or a pharmaceutically acceptable salt thereof.

20. The polysaccharide polymer of claim 1, wherein the polysaccharide polymer comprises one of a compound of Formula (IV), (V), (VI), or (VII), or a pharmaceutically acceptable salt thereof.

21. The polysaccharide polymer of claim 1, wherein the polysaccharide polymer comprises two of a compound of Formula (IV), (V), (VI), or (VII), or a pharmaceutically acceptable salt thereof.

22. The polysaccharide polymer of claim 1, wherein the compound of Formula (I) has a structure selected from Table 3, or a pharmaceutically acceptable salt thereof.

23. The polysaccharide polymer of claim 1, wherein the compound of Formula (I) is selected from Compound 100, Compound 101, Compound 110, Compound 112, Compound 113, Compound 114, Compound 122, and Compound 123, or a pharmaceutically acceptable salt thereof.

24. The polysaccharide polymer of claim 1, wherein the compound of Formula (I) is Compound 101 or a pharmaceutically acceptable salt thereof.

25. The polysaccharide polymer of claim 1, wherein the polysaccharide polymer is alginate, the crosslinking moiety is selected from a compound listed in Table 4 or a pharmaceutically acceptable salt thereof, and the compound of Formula (I) is Compound 101 or a pharmaceutically acceptable salt thereof.

26. A composition comprising a polysaccharide polymer of any one of claims 1-25.

27. A hydrogel capsule comprising a polysaccharide polymer of any one of claims 1-25.

28. The hydrogel capsule of claim 27, wherein the hydrogel capsule comprises a single compartment comprising the polysaccharide polymer (e.g., a polysaccharide polymer described herein).

29. The hydrogel capsule of claim 27, wherein the hydrogel capsule comprises a plurality of compartments, wherein one of the compartments comprises the polysaccharide polymer (e.g., a polysaccharide polymer described herein).

30. The hydrogel capsule of claim 29, wherein the hydrogel capsule comprises an inner compartment and an outer compartment.

31. The hydrogel capsule of claim 30, wherein:
the inner compartment comprises a first polysaccharide polymer comprising the crosslinking moiety;
the outer compartment comprises a second polysaccharide polymer comprising the crosslinking moiety.

32. A hydrogel capsule comprising:
(i) an inner compartment comprising a first polysaccharide polymer comprising a compound of Formula (I): 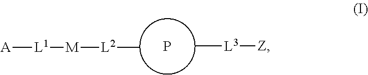
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆-alkylene)-, —N(R^{C})C(O)(C₂-C₆-alkenylene)-, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4; and
(ii) an outer compartment comprising a second polysaccharide polymer comprising a crosslinking moiety.

33. The hydrogel capsule of claim 32, wherein the polysaccharide polymer (e.g., the first polysaccharide polymer and/or the second polysaccharide polymer) is selected from alginate, hyaluronate, and chitosan.

34. The hydrogel capsule of claim 32, wherein the polysaccharide polymer (e.g., the first polysaccharide polymer and/or the second polysaccharide polymer) is alginate.

35. The hydrogel capsule of claim 32, wherein the first polysaccharide polymer is alginate.

36. The hydrogel capsule of claim 32, wherein the second polysaccharide polymer is alginate.

37. The hydrogel capsule of claim 32, wherein the alginate is a high guluronic acid (G) alginate or a high mannuronic acid (M) alginate.

38. The hydrogel capsule of claim 32, wherein the crosslinking moiety has a structure of Formula (IV): 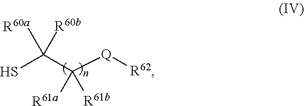
or a pharmaceutically acceptable salt or tautomer thereof, wherein:
Q is O, NR³³, or C(R^{34a})(R^{34b});
each of R³³, R^{34a}, R^{34b}, R^{60a}, R^{60b}, R^{61a}, R^{61b}, and R⁶² is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

39. The hydrogel capsule of claim 32, wherein the crosslinking moiety has a structure of Formula (V): 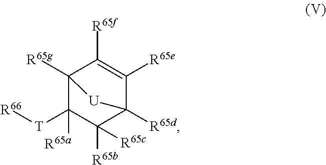
or a pharmaceutically acceptable salt or tautomer thereof, wherein:
each of T and U is independently O, NR³³, or C(R^{34a})(R^{34b});
each of R³³, R^{34a}, R^{34b}, R^{65a}, R^{65b}, R^{65c}, R^{65a}, R^{65e}, R^{65f}, R^{65g} and R⁶⁶ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloakyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

40. The hydrogel capsule of claim 32, wherein the crosslinking moiety has a structure of Formula (VI): 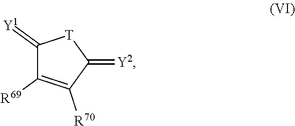
or a pharmaceutically acceptable salt or tautomer thereof, wherein:
each of T, Y¹, and Y² is independently O, NR³³, or C(R^{34a})(R^{34b});
each of R³³, R^{34a}, R^{34b}, R⁶⁹, and R⁷⁰ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}) N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

41. The hydrogel capsule of claim 32, wherein the crosslinking moiety has a structure of Formula (VII): 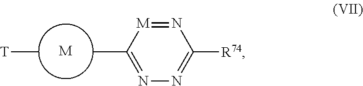
or a pharmaceutically acceptable salt or tautomer thereof, wherein:
T is O, NR³³, or C(R^{34a})(R^{34b});
Ring M is cycloalkyl, heterocyclyl, aryl, heteroaryl, each of which is optionally substituted with 1-6 R⁷;
each of R³³, R^{34a}, R^{34b} and R⁷⁴ is independently hydrogen, alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl;
each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; and
each R⁷ is independently alkyl, alkenyl, alkynyl, heteroalkyl, halogen, cyano, oxo, hydroxyl, cycloalkyl, or heterocyclyl.

42. The hydrogel capsule of claim 32, wherein the compound of Formula (IV), (V), (VI), or (VII) is selected from a compound in Table 4 or a pharmaceutically acceptable salt thereof.

43. The hydrogel capsule of claim 32, wherein the compound of Formula (I) has a structure selected from Table 3, or a pharmaceutically acceptable salt thereof.

44. The hydrogel capsule of claim 32, wherein the compound of Formula (I) is selected from Compound 100, Compound 101, Compound 110, Compound 112, Compound 113, Compound 114, Compound 122, and Compound 123, or a pharmaceutically acceptable salt thereof.

45. The hydrogel capsule of claim 32, wherein the compound of Formula (I) is Compound 101 or a pharmaceutically acceptable salt thereof.

46. The hydrogel capsule of claim 32, wherein the hydrogel capsule has a diameter of between 0.1 mm to 5 mm

47. The hydrogel capsule of claim 32, wherein the hydrogel capsule has a diameter of between 1 mm to 5 mm.

48. The hydrogel capsule of claim 32, wherein the hydrogel capsule has a diameter of between 1 mm to 2.5 mm.

49. The hydrogel capsule of claim 32, wherein the hydrogel capsule encapsulates a cell.

50. The hydrogel capsule of claim 49, wherein the cell produces a therapeutic agent.

51. The hydrogel capsule of claim 50, wherein the therapeutic agent is a protein, e.g., a hormone, a blood clotting factor, an antibody, or an enzyme.

52. The hydrogel capsule of claim 32, wherein the hydrogel capsule is formulated for implantation into a subject (e.g., into the intraperitoneal (IP) space, the peritoneal cavity, the omentum, the lesser sac, the subcutaneous fat).

53. The hydrogel capsule of claim 32, wherein the implantable element is formulated for implantation into the IP space of a subject.

54. A composition comprising a hydrogel capsule of any one of claims 32-53.

55. A method of producing a hydrogel capsule comprising a polysaccharide polymer of any one of claims 1-25.

56. A method of increasing the stability of a hydrogel capsule comprising polysaccharide polymers, wherein the method comprises providing a means of both ionically crosslinking the polysaccharide polymers and covalently crosslinking the polysaccharide polymers.

57. The method of claim 56, wherein the means of ionically crosslinking the polysaccharide polymers comprises use of a divalent cation (e.g., Ba²⁺, Ca²⁺, Sr²⁺).

58. The method of any one of claims 56-57, wherein the means of covalently crosslinking the polysaccharide polymers comprises use of a crosslinking moiety.

59. A method for treating a disease, disorder, or condition in a subject comprising administering to the subject a hydrogel capsule of any one of claims 32-53 or a composition of claim 54, thereby treating the disease, disorder, or condition in the subject.

60. The method of claim 59, wherein the disease, disorder, or condition is diabetes (e.g., Type 1 diabetes).

61. The method of claim 59, wherein the disease, disorder, or condition is not diabetes (e.g., Type 1 diabetes).

62. The method of claim 59, wherein the subject is a human.
